(19) United States
(12) Patent Application Publication
(10) Pub. No.: US 2008/0249075 A1
(43) Pub. Date: Oct. 9, 2008

(54) C11 Modified Retrosteroids as Progesterone Receptor Modulator Compounds

(75) Inventors: Josef Messinger, Sehnde (DE); Christiane Boecker, Hannover (DE); Heinrich-Hubert Thole, Hannover (DE); Bettina Husen, Hannover (DE); Maria Hinaje, Nancy (FR); Monika Buchholz, Langenfeld (DE)

Correspondence Address: CROWELL & MORING LLP;INTELLECTUAL PROPERTY GROUP, P.O. BOX 14300, WASHINGTON, DC, 20044-4300, US

(73) Assignee: Solvay Pharmaceuticals GmbH, Hannover (DE)

(21) Appl. No.: 12/051,937

(22) Filed: Mar. 20, 2008

(30) Foreign Application Priority Data
Mar. 22, 2007 (EP) 07104672.6

(51) Int. Cl. A61K 31/58; C07J 7/00; A61K 31/57; C12Q 1/00; A61P 15/00; C12Q 1/02; C07J 43/00

(52) U.S. Cl. 514/172; 552/590; 514/179; 540/113; 435/29; 435/4

(57) ABSTRACT

Retrosteroidal compounds corresponding to formula I, representing progesterone receptor modulators, and their production, and pharmaceutical preparations containing these compounds. These compounds are useful in the treatment of benign gynecological disorders such as endometriosis and uterine fibroids, as well as for female birth control and for hormone replacement therapy.

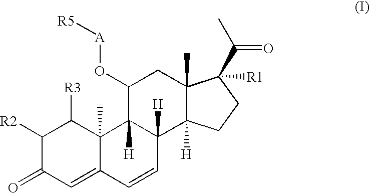

### FIELD OF INVENTION

The present invention relates to novel retrosteroidal derivatives that may be modulators (i.e., agonists, partial agonists and antagonists) of progesterone receptors, to their salts, to pharmaceutical preparations containing these compounds, to processes for the preparation of these compounds, and to uses of said compounds. The invention relates to the use of a compound disclosed herein for the manufacture of a medicament giving a beneficial effect, whereby a beneficial effect is disclosed herein or apparent to a person skilled in the art from the specification and general knowledge in the art. The invention also relates to the use of a compound of the invention for the manufacture of a medicament for treating or preventing a disease or condition. More particularly, the invention relates to a new use for the treatment of a disease or condition disclosed herein or apparent to a person skilled in the art from the specification and general knowledge in the art. In embodiments of the invention specific compounds disclosed herein are used for the manufacture of a medicament useful in the treatment of disorders or conditions mediated by progesterone receptors, or of disorders or conditions that can be treated via modulation of those receptors. In particular, the invention concerns the therapeutic use of said novel retrosteroidal derivatives in the treatment or prevention of benign gynecological disorders, especially endometriosis, uterine fibroids, and dysfunctional uterine bleeding, in hormonal female contraception or in hormone replacement therapy.

### BACKGROUND OF THE INVENTION

The publications and other materials used herein to illuminate the background of the invention, and in particular, cases to provide additional details respecting the practice, are incorporated by reference herein and are not admitted to be prior art.

### Progesterone and the Progesterone Receptor

Progesterone is secreted in large amounts from the ovary or the placenta during the cycle and in pregnancy. In combination with estrogens, progesterone produces cyclic changes of the mucous membrane of the uterus in the menstrual cycle. In pregnancy, progesterone controls the relaxation of the myometrium and preserves the function of the decidual tissue. Under the influence of elevated progesterone levels after ovulation, the mucous membrane of the uterus is converted into a state that allows the nidation of an embryo (blastocyst). In a subtle way, progesterone is involved in the control of ovulation processes. It is known that progesterone has anti-ovulatory properties in connection with estrogens. The latter finding results from an inhibition of the hypophyseal gonadotropin secretion, which is a requirement for the maturation of a follicle and its ovulation. In contrast, it is evident that the comparatively low progesterone secretion of the maturing follicle plays an active role for the preparation and triggering of ovulation. In this connection, hypophyseal mechanisms (time-limited so-called positive feedback of progesterone on gonadotropin secretion) play a significant role. In addition, it is known that progesterone exerts a decisive influence on the endometrium. The endometrial proliferation is inhibited by the suppression of the estrogen-mediated mitosis in the uterus tissue.

### PR Modulators and SPRMs

Within the scope of the present invention, progesterone receptor (PR) modulators comprise compounds which may be agonists showing high affinity and/or high specificity, partial agonists (i.e., partial activators and/or tissue-specific activators) and/or antagonists for PRs, whereby the term PR always comprises the progesterone receptor alpha (PRα) and/or the progesterone receptor beta (PRβ) isoforms. Generally spoken is a compound that binds to the PR and mimics the action of the natural hormone, i.e. progesterone, termed an agonist, whilst a compound which inhibits the effect of said natural ligand is an antagonist. Preferably, the (selective) PR modulators—usually called SPRMs—possess both agonistic and antagonistic activities at the PR measured in-vitro, e.g. using assays of progesterone dependent enzymes in PR expressing cell lines, and/or determined in vivo, e.g. using the classical bioassay, the McPhail test, which assesses progestagenic and antiprogestagenic effects in rabbits [McPhail, 1934ⁱ]. A typical in-vitro assay to determine agonistic and antagonistic activities of the compounds at the PR is the so-called “AP assay” (a progesterone-dependent endogenous alkaline phosphatase (AP) expression assay) using the human mammary carcinoma T47D cell line [Di Lorenzo, 1991ⁱⁱ and Sobek, 1994ⁱⁱⁱ].

An even more sophisticated definition of SPRMs—as mesoprogestins—is given within international patent application WO 01/15679^{iv}: As combined progestins (PR agonist) and anti-progestins (PR antagonists), mesoprogestins show high binding affinity to PR, but exhibit different pharmacodynamic properties compared to either pure progestins or antiprogestins. Mesoprogestins possess progesterone agonistic activity which can be measured in vitro or in commonly used biological tests in vivo; however, this activity remains below that of natural progesterone in the plateau of the dose response curve. Accordingly, mesoprogestins stabilize the function of the PR at an intermediate activity level providing the rationale for the different clinical applications in gynecological therapy.

In the classical bioassay, the McPhail test, which assesses progestagenic and antiprogestagenic effects in rabbits [McPhail, 1934ⁱ], progesterone produces a maximum McPhail score of 4 (by definition). According to the definition given within WO 01/15679^{iv}, the treatment with a mesoprogestin in the absence of progesterone leads, however, to a McPhail score which is higher than that under any dose of RU 486 (Mifepristone), i.e. above 0.5-1, preferentially above 2-0, but to a distinctly lower score than 4 at the plateau of the dose response curve at the clinically relevant doses (i.e. 0.01 mg-30 mg/rabbit). The capacity of mesoprogestins to antagonize progesterone function can also be tested in the McPhail test using a progesterone dose which induces a McPhail score ranging between 3 and 4. A SPRM inhibits the effect of progesterone to a significant degree, but the maximum inhibition is below that which is inducible with RU 486 or other pure antiprogestins, such as onapristone.

### Preferred Indications

PR modulators have been widely used in regulation of female reproduction systems and in treatment of female hormone dependent diseases (e.g. reviewed in Spitz [Steroids, 2003^{v}]. In particular, benign gynecological pathologies such as endometriosis, uterine leiomyomas (uterine fibroids or myomas), adenomyosis, dysfunctional uterine bleeding (menorrhagia and metrorrhagia) and dysmenorrhoea can be treated by the administration of PR modulators. Furthermore, SPRMs may also be useful for the treatment of endometrial hyperplasia, meningiomas, hormone-dependent cancers such as ovarian cancer, breast cancer, endometrial cancer and prostate cancer and female osteoporosis. SPRMs can also be used for female hormone replacement therapy, i.e. for the treatment of hormonal disorders in postmenopausal women such as e.g. hot flushes and/or mood disorders. In addition, SPRMs can be used in female contraceptives.

Endometriosis is a well-known gynaecological disorder that affects 10 to 15% of women in the reproductive age. It is a benign disease defined as the presence of viable endometrial gland and stroma cells outside the uterine cavity. It is most frequently found in the pelvic area. In women developing endometriosis, the endometrial cells entering the peritoneal cavity by retrograde menstruation (the most likely mechanism) have the capacity to adhere to and invade the peritoneal lining, and are then able to implant and grow. The implants respond to steroid hormones of the menstrual cycle in a similar way as the endometrium in the uterus. The infiltrating lesions and the blood from these lesions which are unable to leave the body cause inflammation of the surrounding tissue. The most common symptoms of endometriosis are primary or acquired dysmenorrhoea, dyspareunia and (chronic) pelvic pain, especially before and in the menstruation period. Further symptoms could include dysuria, various genitourinary symptoms secondary to urethral obstruction and/or bladder invasion, painful defecation, rectal pressure, defecation urgency and bowel obstruction, bleeding abnormalities, including menorrhagia or metrorrhagia, infertility, primary or secondary, recurrent spontaneous abortions. The occurrence of these symptoms is not related to the extent of the lesions. Some women with severe endometriosis are asymptomatic, while women with mild endometriosis may have severe pain. Up to now, no reliable non-invasive test is available to diagnose endometriosis. Laparoscopy has to be performed to diagnose the disease. Endometriosis is classified according to the 4 stages set up by the American Fertility Society (AFS). Stage I corresponds to minimal disease while stage 1V is severe, depending on the location and the extent of the endometriosis. Endometriosis is found in up to 50% of the women with infertility. However, currently no causal relation has been proven between mild endometriosis and infertility. Moderate to severe endometriosis can cause tubal damage and adhesions leading to infertility. The aims of treatment of endometriosis are pain relief, resolution of the endometriotic tissue and restoration of fertility (if desired). The two common treatments are surgery or anti-inflammatory and/or hormonal therapy or a combination thereof.

Uterine leiomyomas (fibroids or myomas), benign clonal tumours, arise from smooth muscle cells of the human uterus. They are clinically apparent in up to 25% of women and are the single most common indication for hysterectomy. They cause significant morbidity, including prolonged and heavy menstrual bleeding, pelvic pressure and pain, urinary problems, and, in rare cases, reproductive dysfunction. The pathophysiology of myomas is not well understood. Myomas are found submucosally (beneath the endometrium), intramurally (within the myometrium) and subserosally (projecting out of the serosal compartment of the uterus), but mostly are mixed forms of these 3 different types. The presence of sex steroid receptors in leiomyoma cells has been studied by Tamaya et al. [1985]^{vi}. They have shown that the ratios of estrogen receptor compared to progesterone and androgen receptor levels were higher in leiomyomas than in the corresponding normal myometrium. Surgery has long been the main treatment for myomas. Furthermore, medical therapies that have been proposed to treat myomas include administration of a variety of steroids such as the androgenic steroids danazol or gestrinone, GnRH agonists and progestogens, whereby the administration is often associated with a variety of serious side-effects.

Dysfunctional uterine bleeding disorders (dysfunctional or abnormal uterine bleeding, metrorrhagia and menorrhagia, hypermenorrhea) are forms of pathological bleeding that are not attributable to organic changes in the uterus (such as, e.g., endometrial carcinoma, myomas, polyps, etc.), systemic coagulation disorders, or a pathological pregnancy (e.g., ectopic pregnancy, impending abortion) [American College of Obstetricians and Gynecologists, 1982]. The average blood loss during normal menstruation is about 30 ml, whereby the period lasts for an average of 5 days. If the blood loss exceeds 80 ml, it is classified as pathological [Zahradnik, 1992^{vii}]. Metrorrhagias are defined as bleeding that may or may not be accompanied by pain and that cannot be linked to menstruation or cycle. If it lasts over 7 days, the blood loss often exceeds 80 ml. Menorrhagia is menstruation that may or may not be accompanied by pain, normally every 27-28 days, which, when it lasts over 7 days, is associated in most cases with an increased blood loss of over 80 ml. Menorrhagia is a syndrome of unknown origin and one of the most common problems in gynecology. 60% of women refereed with menorrhagia have a hysterectomy within five years. Hypermenorrhea is defined as menstruation that may or may not be accompanied by pain, normally every 27-28 days for 4-5 days with an elevated blood loss of over 80 ml, sometimes even defined as associated with an increased blood loss of over 150 ml. Forms of dysfunctional uterine bleeding (mainly metrorrhagias and menorrhagias) are typical of adolescence and of the time of menopause, in which follicle-stimulating disorders, anovulation, and yellow-body and follicle persistence occur in clusters. The incidence of dysfunctional uterine bleeding is high and represents one of the most frequent reasons for gynecological consultation for women of reproductive age. The consultation rate because of dysfunctional uterine bleeding is 33% in reproductive age and 69% in perimenopause and postmenopause [Mencaglia et al. 1987^{viii}].

Everything that has been said above in relation to the treatment of uterine leiomyomas, endometriosis and dysfunctional uterine bleeding, equally applies to other benign gynaecological disorders, notably adenomyosis and dysmenorrhea. These benign gynaecological diseases can be treated in a comparable way as described herein before in relation to uterine leiomyomas, endometriosis and dysfunctional uterine bleeding. The available pharmaceutical treatments, however, suffer from the same major drawbacks, i.e. they have to be discontinued once the side-effects become more serious than the symptoms to be treated and symptoms reappear after discontinuation of the therapy.

### Known Compounds Acting as SPRMs

Several PR modulators of steroidal origin are known in the literature and have been recently reviewed, see e.g. Spitz [2005]^{ix}, Spitz [Steroids, 2003]^{v}, Spitz [Expert Opin Invest Drugs, 2003]^{x}. Non-steroidal PR modulators have been reviewed by Zhang et al. [2003]^{xi}.

The so far best characterized PR modulator of steroidal origin is Asoprisnil (J-867)

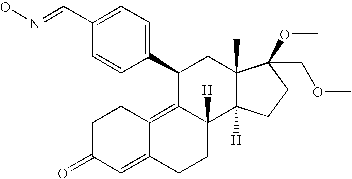

This compound belongs to the class of 11β-benzaldoxime-substituted estratrienes that exhibit partial progesterone agonist/antagonist effects with high PR specificity in animals and humans [Schubert et al., 2005^{xii}]. Asoprisnil (J867) has been described to be under development for the potential oral treatment of uterine fibroids and endometriosis.

The 11β-benzaldoxime-substituted estratrienes having the general structure shown below, in which R can be a hydrogen atom or an alkyl group and R1 can be a hydrogen atom, an alkyl group or aryl group or an optionally substituted acyl function, are known as PR modulators from EP 1229906^{xiii} and EP 0648778^{xiv}:

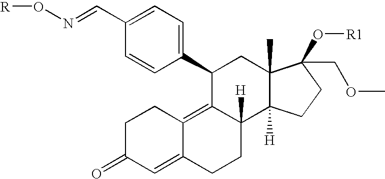

WO 99/45023^{xv} relates to S-substituted 11β-benzaldoxim-estra-4,9-diene-carboxylic acid-thiol ester. The compounds have antigestagenic properties while at the same time having an antigluocorticoidal action that is significantly more reduced in comparison to that of RU 486.

In EP 909764^{xvi}, 11β-benzaldoxime-9α,10α-epoxy-estr-4-ene derivatives with high binding affinity to the PR in the presence of low glucocorticoid receptor affinity are described.

WO 01/44267^{xvii} describes new 11β-phenylestradiene derivatives with fluoroalkyl groups in the aromatic side chain and production thereof. The compounds or the pharmaceutical preparations that contain these compounds are antihormonally effective and are therefore suitable for the treatment of diseases that are unfavorably influenced by cortisol or by corticoids, for the reduction of secreted cortisol, for stimulation of lactation, for treating dysmenorrhea and myomas, for treating Cushing's disease and for cervical maturation, for improving cognitive performance, for treating endometriosis or for hormone replacement therapy (HRT).

WO 03/093292^{xviii} discloses 17α-fluoroalkyl-11β-benzaldoxime-steroids and production thereof, pharmaceutical preparations that contain these steroids, especially for postmenopausal substitution therapy of gynecological diseases, such as hysteromyomas or dysmenorrhoic symptoms.

WO 04/014935^{xix} describes further substituted 11β-benzaldoxime-steroids, in particular 4-(3-oxo-estra-4,9-dien-11 beta-yl)-benzaldehyde oximes, which are PR modulators useful in female contraception, hormone replacement therapy and treatment of gynecological disorders.

Further 11β substituted 17β-acyl-17α-propynyl steroids are known from WO 01/18025, whereas WO 00/34306^{xxi} describes 17β-acyl-17α-propynyl-11β-arylsteroids and their derivatives having agonist or antagonist hormonal properties. In addition, WO 99/62928^{xxii} discloses 17β-amino- and 17β-hydroxylamino-11β-arylsteroids, WO 99/62929^{xxiii} discloses 17β-nitro-11β-aryl-steroids and WO 99/45022^{xxiv} discloses 20-keto-11β-arylsteroids having agonist or antagonist hormonal properties.

The effectiveness of known steroidal SPRMs is often tempered by their undesired side-effect profile, particularly during long-term administration. For example, the effectiveness of synthetic progestins, such as Norgestrel, as female birth control agents must be weighed against the increased risk of breast cancer and heart disease. Similarly, the progesterone antagonist, mifepristone (RU 486), if administered for chronic indications, such as uterine fibroids, endometriosis and certain hormone-dependent cancers, could lead to homeostatic imbalances in a patient due to its inherent cross-reactivity as a glucocorticoid receptor (GR) antagonist.

Accordingly, identification of compounds which have good receptor-selectivity for the PR over other steroid hormone receptors, which provide a good tissue-selectivity (e.g. selectivity for uterine tissue over breast tissue) and which are agonists, partial agonists (i.e., partial activators and/or tissue-specific activators) and/or antagonists for PRs, which preferably show a balanced agonistic/antagonistic profile, would be of significant value in the improvement of women's health.

### Known Retrosteroids

Retrosteroids, i.e. steroids with 9β,10α conformation, are well known in the state of the art. The commercially available compound Dydrogesterone ((9β,10α)-Pregna-4,6-diene-3,20-dione) of the following formula

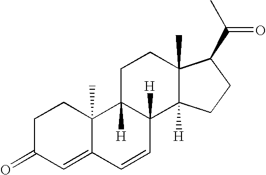

is an orally active progestative hormone and is generally used to correct deficiencies of progesterone in the body. The synthesis of Dydrogesterone by irradiation and photochemical reaction is for example described within European patents EP 0152138B1^{xxv} (U.S. Pat. No. 4,601,855) and EP 0558119B1^{xxvi} (U.S. Pat. No. 5,304,291).

Further known retrosteroids with progestational activity are for example 1,2-methylene-3-keto-Δ^{4,6}-bisdehydro-6-halo-9β,10α-steroids as disclosed within U.S. Pat. No. 3,937,700^{xxvii} and 3-keto-Δ^{4,6}-bisdehydro-6-halo-9β,10α-steroids as described within BE 652,597^{xxviii} and U.S. Pat. No. 3,304,314^{xxix}. Furthermore, the U.S. Pat. No. 3,555,053^{xxx} describes a process for the preparation of 6-halo- or 6-alkyl-9β,10α-steroids. Some 6,7-dehydro-9β,10α steroids are described by Westerhof & Hartog [1965]^{xxxi}. The synthesis of further retrosteroids is disclosed within Hartog et al. [1972]^{xxxii} for some 16-methylene-17α-acetoxy-9β,10α-pregna-4,6-diene-3,20-dione derivatives and within Halkes et al [1972]^{xxxiii} for 1,2β-methylene-17α-acetoxy-9β,10α-pregnanes. In addition, 18-alkyl-9β,10α-pregnane derivatives are disclosed by Van Moorselaar & Halkes [1969]^{xxxiv}. However, the retrosteroidal compounds known so far were all developed for having progestational activity, i.e. being PR agonists.

Further retrosteroids carrying hydroxy or esterified hydroxy substituents in the C11 position were already described within GB 1,111,320^{xxxv}. Compounds or intermediates especially described are 11β-Hydroxy-9β,10α-pregna-4,6-diene-3,20-dione (CAS No. 22413-62-3), 11-Hydroxy-9β,10α-pregna-4-ene-3,20-dione (CAS No. 10007-43-9), and 11β-17α-Dihydroxy-9β,10α-pregna-4-ene-3,20-dione (CAS No. 4076-89-5), as well as the 11β-acetoxy derivatives thereof, namely 11β-Acetoxy-9β,10α-pregna-4-ene-3,20-dione, and 11β-Acetoxy-9β,10α-pregna-4,6-diene-3,20-dione (CAS No. 22393-79-9), which were obtained from the corresponding 11β-hydroxy compounds by chemical modification. However, GB 1,111,320^{xxxv} only generally states that the steroids described therein may be hormonally (e.g. progestatively, anabolically or corticoidally) active, without providing any data supporting this hypothesis.

Since compounds which are agonists, partial agonists (i.e., partial activators and/or tissue-specific activators) and/or antagonists for progesterone receptors, preferably showing a balanced agonistic/antagonistic profile, are regarded to be of significant value for the improvement of women's health, there still remains a need for the development of novel compounds which therapeutically modulate the progesterone receptor with an improved agonistic and/or antagonistic mode, which show with higher receptor-selectivity for the progesterone over other steroid hormone receptors than currently known compounds, and which provide a good tissue-selectivity (e.g. selectivity for uterine tissue over breast tissue). Retrosteroidal compounds carrying different kind of substituents in the C-11β position might fulfill this aim. However, despite the compounds disclosed in GB 1,111,320^{xxxv} no retrosteroidal derivatives carrying substituents in the 11β position have been disclosed so far.

Accordingly, there remains still a need for the development of novel compounds which therapeutically modulate the PR with an improved agonistic and/or antagonistic mode and with higher selectivity than currently known compounds. In particular, there is a need for selective PR modulators useful for the treatment of benign gynecological disorders such as endometriosis, uterine fibroids, uterine leiomyoma, endometrial hyperplasia, dysmenorrhea, and dysfunctional uterine bleeding (menorrhagia, metrorrhagia).

### SUMMARY OF THE INVENTION

An object of the present invention was to provide novel PR modulators based on the retrosteroidal core of the known progesterone agonist Dydrogesterone.

Another object of the invention is to provide compounds that combine the known beneficial properties of Dydrogesterone with novel modifications of the retrosteroidal core in order to obtain PR modulators, i.e. compounds with agonistic as well as antagonistic properties towards the PR, suited for the treatment of a broad range of gynaecological diseases requiring the modulation of the PR.

Surprisingly it has been found that the compounds of the invention represent PR modulators possessing agonistic and/or antagonistic activities at the PR in vivo. Accordingly, the present invention relates to compounds of general formula (I):

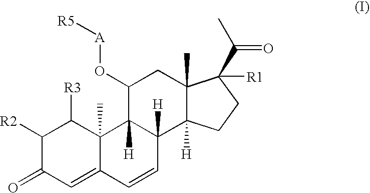

wherein
A represents —CO—, —CO—NR6-, —CO—NR4-NR6- or —CO—NH—SO₂—NR6-;
R1 is selected from hydrogen, —OH, —O—(C₁-C₄)alkyl and —O—CO—(C₁-C₄)alkyl;
R2 and R3 are both hydrogen or together form a methylene group;
R4 is selected from hydrogen and —(C₁-C₄)alkyl;
R5 is selected from
   (a) aryl and aryl-(C₁-C₄)alkyl, wherein
      (i) the aryl group is unsubstituted; or
      (ii) the aryl moiety of the aryl-(C₁-C₄)alkyl group is unsubstituted, or
      (iii) the aryl moiety of the aryl or aryl-(C₁-C₄)alkyl group is substituted with one or more substituents independently selected from —O—R⁹, —S—R⁹, —O—CO—NHR¹⁰, —O—CO—R¹¹, —CO—R¹¹, —SO₂—R¹¹, —CO—O—R⁹, —CO—NR⁷R⁸, —SO₂—NR⁷R⁸, —CN, —CH═N—O—R¹², —CH═N—O—CO—NHR¹⁰, —CH═N—O—CO—R¹¹, —CH═N—O—CO—O—R⁹, —(C₁-C₄)alkyl-CO—O—R⁹, —NR⁷R⁸, —NR¹³—CO—R¹¹, —NR¹³—CO—NHR¹⁰, —NR¹³—CO—O—R⁹, —NR¹³—SO₂—R¹¹, halogen, —(C₁-C₄)alkyl, halogenated —(C₁-C₄)alkyl, cycloheteroalkyl, aryl and heteroaryl, the number of said substituents being 1, 2, 3, 4 or 5 for halogen, and 1, 2 or 3 for any combination of said substituents, and wherein the cycloheteroalkyl or heteroaryl is optionally substituted with one or two substituents independently selected from oxo, halogen, —(C₁-C₄)alkyl and halogenated —(C₁-C₄)alkyl; or
      (iv) the aryl moiety of the aryl or aryl-(C₁-C₄)alkyl group is substituted by two groups which are attached to adjacent carbon atoms and are combined into a saturated or partly unsaturated cyclic 5-, 6-, 7- or 8-membered ring system, optionally containing 1, 2 or 3 heteroatoms selected from N, O and S, the number of N atoms being 0, 1, 2 or 3 and the number of O and S atoms each being 0, 1 or 2; whereby the cyclic ring system is optionally substituted by one or two substituents independently selected from oxo, —(C₁-C₄)alkyl and halogenated —(C₁-C₄)alkyl;
   (b) heteroaryl and heteroaryl-(C₁-C₄)alkyl,
      wherein the heteroaryl moiety of the heteroaryl or heteroaryl-(C₁-C₄)alkyl group is optionally substituted with one or more substituents independently selected from —O—R⁹, —S—R⁹, —O—CO—NHR¹⁰, —CO—R¹¹, —O—CO—R¹¹, —SO—R¹¹, CO—O—R⁹, —CO—NR⁷R⁸, —SO₂—NR⁷R⁸, —CN, —CH═N—O—R¹², —CH═N—O—CO—NHR¹⁰, —CH═N—O—CO—R¹¹, —CH═N—O—CO—O—R⁹, —(C₁-C₄)alkyl-CO—O—R⁹, —NR⁷R⁸, —NR¹³—CO—R¹¹, —NR¹³—CO—NHR¹⁰, —NR¹³—CO—O—R⁹, —NR¹³—SO₂—R¹¹, halogen, —(C₁-C₄)alkyl, halogenated —(C₁-C₄)alkyl and aryl, the number of said substituents being 1, 2, 3, 4 or 5 for halogen, and 1, 2 or 3 for any combination of said substituents;
   (c) cycloheteroalkyl and cycloheteroalkyl-(C₁-C₄)alkyl,
      wherein the cycloheteroalkyl moiety of the cycloheteroalkyl or cycloheteroalkyl-(C₁-C₄)alkyl group is optionally substituted with 1, 2, 3 or 4 substituents independently selected from the group consisting of oxo, —(C₁-C₄)alkyl and aryl; and
   (d) (C₁-C₈)alkyl, wherein the (C₁-C₈)alkyl group is
      (i) unsubstituted, or
      (ii) substituted with one or two substituents independently selected from the group consisting of halogen, —O—R⁹, —S—R⁹, —NR⁷R⁸, —CO—OR⁹, —CO—R¹¹, —CO—NR⁷R⁸ and —CN;
   wherein the —(C₁-C₄)alkyl moiety of the aryl-(C₁-C₄)alkyl, heteroaryl-(C₁-C₄)alkyl or cycloheteroalkyl-(C₁-C₄)alkyl group in R5 is optionally substituted with one or two substituents independently selected from oxo and hydroxyl;
R6 is selected from hydrogen and —(C₁-C₈)alkyl, optionally substituted with —O—R⁹ or halogen, the number of said substituents being 1, 2 or 3 for halogen, and 1 or 2 for any combination of said halogen or —O—R⁹ moieties; or
R5 and R6 form together with the nitrogen atom, where R5 and R6 are attached, a heterocyclic 5-, 6-, 7- or 8-membered ring system, which is saturated, partly unsaturated, or aromatic; which optionally contains 1, 2 or 3 additional heteroatoms selected from N, O and S, the number of additional N atoms being 0, 1, 2 or 3 and the number of O and S atoms each being 0, 1 or 2; which ring is optionally part of a multiple condensed ring-system; and which ring system is optionally substituted with an aryl group, the aryl group being optionally substituted with one or two substituents independently selected from —O—R¹⁴, halogen, —(C₁-C₄)alkyl and halogenated —(C₁-C₄)alkyl;
wherein R⁷, R⁸, R⁹, R¹⁰, R¹¹, R¹², R¹³ and R¹⁴ are independently selected from the group consisting of hydrogen, —(C₁-C₄)alkyl, halogenated —(C₁-C₄)alkyl, aryl and aryl-(C₁-C₄)alkyl;
   or R⁷ and R⁸ form together with the nitrogen atom, where R⁷ and R³ are attached, a heterocyclic 5-, 6-, 7- or 8-membered ring system, which is saturated, partly unsaturated, or aromatic; and which optionally contains 1 or 2 additional heteroatoms selected from N, O and S, the number of additional N atoms being 0, 1 or 2 and the number of O and S atoms each being 0 or 1;

In one embodiment the substituent R5 of compounds of general formula (I) should only represent unsubstituted —(C₁-C₈)alkyl as defined herewithin under the proviso that A represents —CO—NR6-, —CO-NR4-NR6- or —CO—NH—SO₂—NR6-, i.e. R5 should not represent unsubstituted —(C₁-C₈)alkyl, if A represents —CO—. Alternatively, the substituent R5 of compounds of general formula (I) should only represent optionally substituted —(C₁-C₈)alkyl as defined herewithin under the proviso that A represents —CO-NR6-, —CO—NR4-NR6- or —CO—NH—SO₂-NR6-, i.e. R5 should preferably not represent optionally substituted —(C₁-C₈)alkyl, if A represents —CO—.

In another embodiment, the substituent R5 of compounds of general formula (I) should only represent an unsubstituted aryl group under the proviso that R1 represents —O—(C₁-C₄)alkyl or —O—CO—(C₁-C₄)alkyl; i.e. R5 should not represent an unsubstituted aryl group if R1 represents hydrogen or —OH. Alternatively, the substituent R5 of compounds of general formula (I) should only represent an optionally substituted aryl group under the proviso that R1 represents —O—(C₁-C₄)alkyl or —O—CO—(C₁-C₄)alkyl; i.e. R5 should not represent an optionally substituted aryl group if R1 represents hydrogen or —OH.

The residue A of the compounds of formula (I), and (Ib) is selected from —CO—, —CO-NR6-, —CO-NR4-NR6- and —CO—NH—SO₂—NR6-. In an embodiment, A only represents —CO— under the proviso that R1 is selected from —O—(C₁-C₄)alkyl and —O—CO—(C₁-C₄)alkyl. Alternatively, A only represents —CO— under the proviso that R2 and R3 together form a methylene group.

Pharmaceutically acceptable salts as well as all tautomers, stereoisomers, racemates, enantiomers of the compounds of the invention and mixtures thereof, unless the formula depicting the compound explicitly shows a particular stereochemistry, are also within the scope of the invention. Such isomers can be isolated by standard resolution techniques, including fractional crystallization and chiral column chromatography. Furthermore the compounds of the invention also include isotopically-labeled and radio-labeled compounds, as well as commonly used pro-drugs and active metabolites of these compounds.

Compounds of the invention are also optically pure enantiomers having the formula (Ib)

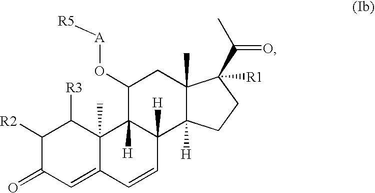

wherein the residues A, R1, R2, R3 and R5 have the meaning as defined herewithin.

Compounds of the invention include those represented by general formulae (II) or (IIb)

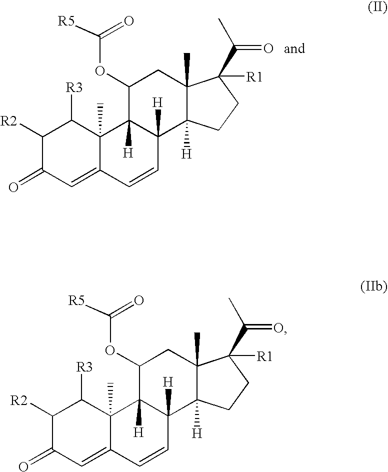

wherein the residues R1, R2, R3 and R5 have the meaning as defined herewithin.

In compounds of formula (II) or (IIb), R1 is selected from hydrogen, —OH, —O—(C₁-C₄)alkyl and —O—CO—(C₁-C₄)alkyl, preferably R1 is selected from —O—(C₁-C₄)alkyl and —O—CO—(C₁-C₄)alkyl, and in particular R1 represents —O—(C₁-C₄)alkyl, R2 and R3 are both hydrogen or together form a methylene group, preferably R2 and R3 together form a methylene group, and all residues R4 through R14 have the same definitions as given above for general formula (I).

The substituent R5 of compounds of general formula (II) or (IIb) preferably does not represent optionally substituted —(C₁-C₈)alkyl, —(C₁-C₆)alkyl or —(C₁-C₄)alkyl.

Further compounds of the invention include those represented by general formula (III) and (IIIb)

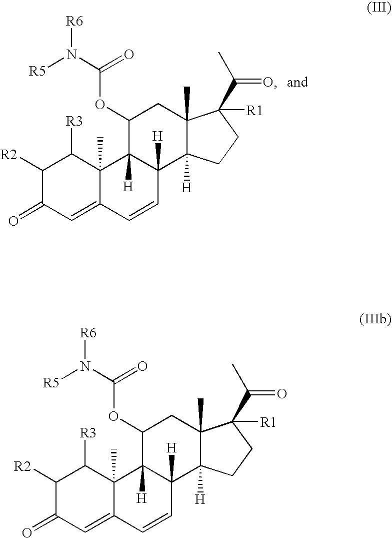

and those represented by general formula (IV) and (IVb)

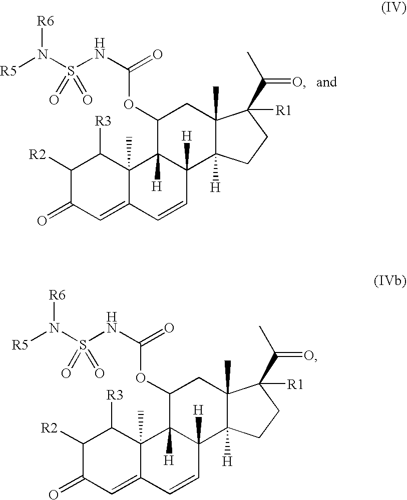

and those represented by general formula (VII) and (VIIb)

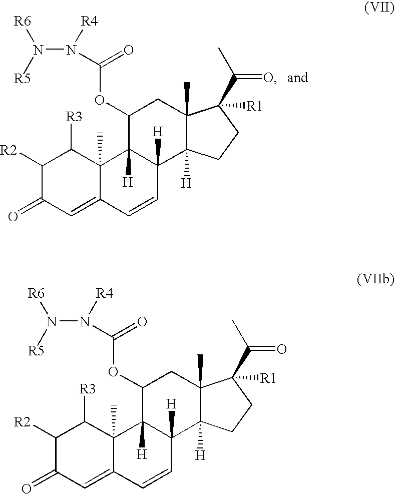

wherein R1 through R14 all have the same definitions as given above for general formula (I).

In a further aspect, the present invention relates to a pharmaceutical composition comprising a pharmacologically active amount of at least one compound of the invention according to any one of formulae I through IV shown above wherein R1 through R14, and n all have the same definitions as given above or as given below for certain embodiments, or a salt or pro-drug thereof, as an active ingredient and at least one pharmaceutically acceptable carrier and/or at least one pharmaceutically acceptable auxiliary substance.

Additionally, the invention relates to a compound of the invention or a salt or pro-drug thereof, for use as a medicament.

Furthermore, the invention relates to the use of a compound of the invention for the manufacture of a medicament for the treatment or prevention of a disorder or condition mediated by a PR, or that can be treated via modulation of that receptor.

In addition, the invention relates to the use of an effective amount of a compound of the invention for the treatment or prevention of a disorder or condition mediated by a PR, or that can be treated via manipulation of that receptor, in an individual, preferably in a mammal, in particular a human.

Preferably the disorder or condition mediated by a PR, or that can be treated via manipulation of that receptor is selected from: endometriosis, uterine fibroids, uterine leiomyoma, endometrial hyperplasia, dysmenorrhea, dysfunctional uterine bleeding, menorrhagia, metrorrhagia, hypermenorrhea, hot flushes, mood disorders, meningiomas, hormone-dependent cancer, in particular female sex steroid dependent cancer, ovarian cancer, breast cancer, endometrial cancer and prostate cancer; female osteoporosis, Cushing's syndrome, major depression, neurodegenerative diseases, Alzheimer's disease, and demyelinating diseases.

In a further aspect, the present invention relates to the use of a compound of the invention for the manufacture of a medicament for female birth control, for modulation of fertility or for female hormone replacement therapy (the treatment of hormonal disorders in postmenopausal women).

Furthermore, it will be understood by those skilled in the art that the compounds of the present invention, including pharmaceutical compositions and formulations containing these compounds, can be used in a wide variety of combination therapies to treat the conditions and diseases described above. Thus, the compounds of the present invention can be used in combination with other hormones, in particular estrogenic compounds and estrogen receptor modulators, and other therapies, including, without limitation, chemotherapeutic agents such as cytostatic and cytotoxic agents, immunological modifiers such as interferons, interleukins, growth hormones and other cytokines, hormone therapies, surgery and radiation therapy.

In particular, the pharmaceutical composition of the invention further comprises at least one low-dose natural or synthetic estrogen or pro-drug thereof; preferably the estrogen is used as a natural estrogen, e.g. as a conjugated estrogen obtained from pregnant mare's urine (conjugated equine estrogen). Alternatively, the estrogen may be presented as the respective 3-sulfamate.

In one embodiment, the pharmaceutical composition of the present invention is in the form of an intrauterine device (IUD), in the form of a transdermal patch or a gel.

Furthermore, the invention also relates to a method of treating an individual, i.e. a mammal such as a human, having a condition mediated by a PR or which condition can be treated via modulation of that receptor, comprising administering to said individual an amount of a compound of this invention, or a salt or a pro-drug thereof, which amount is effective to treat the condition. Administration of compounds of this invention in combination with other pharmaceuticals used in treatment of the listed conditions is contemplated.

The conditions to be treated include but are not limited to endometriosis, uterine fibroids, uterine leiomyoma, endometrial hyperplasia, dysmenorrhea, dysfunctional uterine bleeding, menorrhagia, metrorrhagia, hypermenorrhea, hot flushes, mood disorders, meningiomas, hormone-dependent cancers, in particular female sex steroid dependent cancer, ovarian cancer, breast cancer, endometrial cancer and prostate cancer; female osteoporosis, Cushing's syndrome, major depression, neurodegenerative diseases, Alzheimer's disease, and demyelinating diseases. Additionally, the conditions to be treated may be alleviated with female hormone replacement therapy.

In a further aspect, the present invention relates to a method of modulating fertility (e.g., use of the compounds of the invention as contraceptive agents, contragestational agents or abortifacients, for in vitro fertilization, and for pregnancy maintenance) in an individual comprising administering to said individual a pharmaceutically effective amount of a compound of this invention, or a salt or a pro-drug thereof. Preferably, the present invention provides a method of contraception to an individual comprising administering to said individual a pharmaceutically effective amount of a compound of this invention, or a salt or a pro-drug thereof.

The compounds of the present invention, including pharmaceutical compositions and formulations containing these compounds, may be used in combination or conjunction with one or more estrogenic compounds or estrogen receptor modulators, in particular for female hormone replacement therapy, as modulators of fertility and in treatment of female osteoporosis.

According to a further aspect of the invention, a method is disclosed of modulating a PR in an individual comprising administering to said individual a compound of this invention, or a salt or a pro-drug thereof, in an amount effective to modulate a PR. Preferably, said modulation is activation.

Additionally, the compounds of this invention also have utility when e.g. radio- or isotopically labelled as ligands for use in assays to determine the presence of PR in a cell background or extract. They are particularly useful due to their ability to selectively modulate PRs, and can therefore be used to determine the presence of such receptors in the presence of other steroid receptors or related intracellular receptors. Therefore, the present invention also relates to a method of determining the presence of a progesterone receptor (PR) in a cell or cell extract comprising (a) labeling a compound of this invention, or a salt or a pro-drug thereof; (b) contacting the cell or cell extract with said labeled compound; and (c) testing the contracted cell or cell extract to determine the presence of progesterone receptor.

### DETAILED DESCRIPTION OF THE INVENTION

### Definitions

The following terms are used to describe the present invention. The terms are defined with the following meanings, unless explicitly stated otherwise:

The terms “comprising” and “including” are used herein in their open, non-limiting sense.

The word “compound” shall here be understood to cover any and all isomers (e.g., enantiomers, stereoisomers, diastereomers, rotomers, tautomers) or any mixture of isomers, pro-drugs, and any pharmaceutically acceptable salt of said compound, unless the formula depicting the compound explicitly shows a particular stereochemistry.

Where the plural form is used for compounds, salts, and the like, this is taken to mean also a single compound, salt, or the like.

The term “pro-drug” as used herein, represents derivatives of the compounds of the invention that are drug precursors which, following administration to a patient by any known route, release the more active metabolite drug in vivo via a chemical or physiological process. Pro-drugs are bioreversible derivatives of drug molecules used to overcome some barriers to the utility of the parent drug molecule. These barriers include, but are not limited to, solubility, permeability, stability, presystemic metabolism and targeting limitations [see e.g. Medicinal Chemistry: Principles and Practice, 1994, ISBN 0-85186-494-5, Ed.: F. D. King, p. 215; or Stella, 2004^{xxxvi}, or Ettmayer et al., 2004^{xxxvii}]. In particular, pro-drugs are derivatives of the compounds of the invention in which functional groups carry additional substituents which may be cleaved under physiological conditions in vivo and thereby releasing the active principle of the compound (e.g., a pro-drug on being brought to a physiological pH or through an enzyme action is converted to the desired drug form). Pro-drugs of the compounds mentioned above are also within the scope of the present invention. Pro-drugs that are metabolised to compounds having formula (I), belong to the invention. In particular this relates to compounds with primary or secondary amino or hydroxy groups. Such compounds can be reacted with organic acids to yield compounds having formula (I) wherein an additional group is present which is easily removed after administration, for instance, but not limited to amidine, enamine, a Mannich base, a hydroxyl-methylene derivative, an O-(acyloxymethylene carbamate) derivative, carbamate, ester, amide or enaminone.

Any of the compounds of the present invention can be synthesized as pharmaceutically acceptable salts for incorporation into various pharmaceutical compositions. The term “pharmaceutically acceptable salts” refers to salt forms that are pharmacologically acceptable and substantially non-toxic to the subject being administered the compounds of the invention. Pharmaceutically acceptable salts of compounds of one of the formulae I through IV include conventional and stoichiometrical acid-addition salts or base-addition salts formed from suitable non-toxic organic or inorganic acids or inorganic bases.

Acid addition salts, for example, from compounds of the invention with a basic nitrogen atom are formed preferably with organic or inorganic acids. Suitable inorganic acids include, but are not limited to halogenic acids such as hydrochloric acid, sulfuric acid, or phosphoric acid. Suitable organic acids include, but are not limited to carboxylic, phosphonic, or sulfonic acids, for example acetic acid, propionic acid, glycolic acid, lactic acid, hydroxybutyric acid, malic acid, malei(ni)c acid, malonic acid, nicotinic acid, salicylic acid, fumaric acid, succinic acid, oxalic acid, phenylacetic acid, stearic acid, adipic acid, tartaric acid, citric acid, glutaric acid, 2- or 3-glycerophosphoric acid and other mineral and carboxylic acids well known to those skilled in the art. The salts are prepared by contacting the free base forms with a sufficient amount of the desired acid to produce a salt in the conventional manner.

Compounds of the invention containing acidic substituents may also form salts with inorganic or organic bases. Examples of suitable bases for salt formation include, but are not limited to, inorganic bases such as alkali or alkaline earth-metal (e.g., sodium, potassium, lithium, calcium, or magnesium) hydroxides, and those derived from ammonium hydroxides (e.g., a quaternary ammonium hydroxide such as tetramethylammonium hydroxide). Also contemplated are salts formed with pharmaceutical acceptable amines such as ammonia, alkyl amines, hydroxyalkylamines, N-methylglucamine, benzylamines, piperidines, pyridines, piperazines, and pyrrolidines and the like. Certain compounds will be acidic in nature, e.g. those compounds which possess a carboxyl or phenolic hydroxyl group. Salts of phenols can be made by heating acidic compounds with any of the above mentioned bases according to procedures well known to those skilled in the art.

As used herein, the term “composition” is intended to encompass a product comprising the specified ingredients in the specified amounts, as well as any product which results, directly or indirectly, from combination of the specified ingredients in the specified amounts.

The phrase “effective amount” as used herein, means an amount of a compound or composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically acceptable excipient(s)/carrier(s) utilized, and like factors within the knowledge and expertise of the attending physician.

The term “mediate” means affect or influence. Thus, for example, conditions mediated by a progesterone receptor are those in which a progesterone receptor plays a role. Progesterone receptors are known to play a role in conditions including, for example, infertility, contraception, pregnancy maintenance and termination, female hormone deficiency, dysfunctional uterine bleeding, endometriosis, mood disorder, osteoporosis, and hormone-dependent cancers.

The term “progesterone receptor” as used herein always comprises the progesterone receptor alpha (PRα) and/or the progesterone receptor beta (PRβ) isoforms. Like other steroid hormone receptors, PR is expressed in two isoforms in certain organisms, including humans. Human PRα is a truncated form of human PRβ and lacks 164 amino acids at the N-terminus. Both isoforms are identical in the DNA-binding and ligand-binding domain and induce progestin-mediated gene transcription, but show a somehow different transactivation behavior (see e.g. WO 02/054064^{xxxviii}).

The terms “selective” and “selectivity” refer to compounds that display reactivity towards a particular receptor (e.g. a progesterone receptor) without displaying substantial cross-reactivity towards another receptor (e.g. glucocorticoid receptor, androgen receptor and/or estrogen receptor). Thus, for example, selective compounds of the present invention may display reactivity towards progesterone receptors without displaying substantial cross-reactivity towards other steroid hormone receptors. In one embodiment, a compound of the present invention has at least about 10 fold selectivity to the PR, at least about 50 fold selectivity to the PR, at least about 100 fold selectivity to the PR, at least about 250 fold selectivity to the PR, or at least about 500 fold selectivity to the desired target.

The following terms are used to describe various constituents of the chemical compositions useful in this invention. The terms are defined as follows, unless explicitly stated otherwise:

Any asymmetric carbon atoms may be present in the (R)-, (S)- or (R,S)-configuration, preferably in the (R)- or (S)-configuration, whichever is most active, unless the stereochemistry is explicitly depicted in the corresponding compound formula. Substituents at a double bond or a ring may be present in cis (=Z-) or trans (=E-) form, unless the stereochemistry is explicitly depicted in the corresponding compound formula.

The compounds of the invention have a defined stereochemistry within their steroidal core structure according to the commonly used definition of the configuration of retrosteroids (i.e. steroids with 9β,10α conformation):

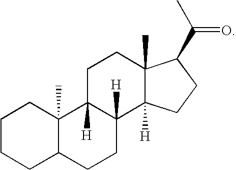

The stereochemistry within the retrosteroidal core structure is always shown in the corresponding compound formula and should not vary within the scope of the present invention, whereas the stereochemistry at the carbon atoms in the steroidal core carrying additional side chains (C1, C2, C3, C11 an C17) and the stereochemistry of any asymmetric carbon atom within the side chains themselves is not fixed unless explicitly depicted. Therefore, the terms “compounds of formula (I)” or “compounds of formula (II)” etc also comprise the stereoisomers of the depicted compounds, unless a particular stereochemistry is explicitly shown within the formula. The stereochemistry shown in the respective formula prevails over the general term “stereoisomers”.

The compounds of the present invention may contain further asymmetric centers on the molecule, e.g. a chiral carbon atom, depending upon the nature of the various substituents. In case of such an asymmetric centre, the compounds could thus be present in two optically active stereoisomeric forms or as a racemate. In certain instances, asymmetry may also be present due to restricted rotation about the central bond adjoining the two aromatic rings of the specified compounds. It is intended that all isomers (including enantiomers and diastereomers), either by nature of asymmetric centers or by restricted rotation as described above, as separated, pure or partially purified isomers or racemic mixtures thereof, be included within the ambit of the instant invention, unless a particular stereochemistry is explicitly depicted in the formula representing a respective compound.

The term “substituted” means that the specified group or moiety bears one or more substituents. Where any group may carry multiple substituents and a variety of possible substituents is provided, the substituents are independently selected and need not to be the same. The term “unsubstituted” means that the specified group bears no substituents. The term “optionally substituted” means that the specified group is unsubstituted or substituted by one or more substituents.

The term “halogen” refers to fluorine (F, Fluoro-), bromine (Br, Bromo-), chlorine (Cl, Chloro), and iodine (J, Iodo-) atoms. The terms “dihalogen”, “trihalogen” and “perhalogen” refer to two, three and four substituents, respectively, each individually selected from the group consisting of fluorine, bromine, chlorine, and iodine atoms.

The term “hydroxyl” refers to the group —OH

The term “oxo” refers to the group ═O

The term “carbamoyl” refers to the group —CO—NH₂

The term “nitrile” or “cyano” refers to the group —CN.

For the purpose of the present invention, the carbon content of various hydrocarbon containing moieties is indicated by a prefix designating the minimum and maximum number of carbon atoms in the moiety, i.e., the prefix Cᵢ-Cⱼ defines the number of carbon atoms present from the integer “i” to the integer “j” inclusive. Thus C₁-C₄-alkyl refers to alkyl of 1-4 carbon atoms, inclusive, or methyl, ethyl, propyl, butyl and isomeric forms thereof.

The term “alkyl” stands for a hydrocarbon radical which may be linear, cyclic or branched, with single or multiple branching, whereby the alkyl group in general comprises 1 to 12 carbon atoms. In one embodiment, the term “alkyl” stands for a linear or branched (with single or multiple branching) alkyl chain of 1 to 8 carbon atoms, exemplified by the term (C₁-C₈)alkyl, more preferably of 1 to 6 carbon atoms exemplified by the term (C₁-C₆)alkyl. The term (C₁-C₈)alkyl is further exemplified by such groups as methyl; ethyl; n-propyl; isopropyl; n-butyl; sec-butyl; isobutyl; tert-butyl; n-pentyl; isopentyl; neopentyl; tert-pentyl; 2- or 3-methylpentyl; n-hexyl; isohexyl, heptyl, octyl and the like. The alkyl or (C₁-C₈)alkyl group may be partially unsaturated, forming such groups as, for example, vinyl, propenyl (allyl), butenyl, pentenyl, pentinyl, hexenyl, octadienyl, and the like. In one embodiment, the term “alkyl” stands for a linear or branched (with single or multiple branching) alkyl chain of 1 to 4 carbon atoms, exemplified by the term (C₁-C₄)alkyl. The term (C₁-C₄)alkyl is further exemplified by such groups as methyl; ethyl; n-propyl; isopropyl; n-butyl; sec-butyl; isobutyl; and tert-butyl. The alkyl or (C₁-C₄)alkyl group may be partially unsaturated, forming such groups as, for example, vinyl, 1-propenyl, 2-propenyl (allyl), and butenyl. The term “alkyl” further comprises cycloalkyl groups, preferably cyclo(C₃-C₈)alkyl which refers to cyclopropyl, cyclobutyl, cyclopentyl, cyclohexyl, cycloheptyl, cyclooctyl and isomeric forms thereof such as methylcyclopropyl; 2- or 3-methylcyclobutyl; 2-, or 3-methylcyclopentyl, and the like. The cycloalkyl group may also be partly unsaturated, forming such groups as, for example, cyclohexenyl, cyclopentenyl, cyclooctadienyl, and the like. Furthermore, the term “alkyl” comprises a cycloalkyl-alkyl group comprising 4 to 12 carbon atoms, preferably “—(C₁-C₄)alkyl-cyclo(C₃-C₈)alkyl” which refers to a alkyl group of 1 to 4 carbon atoms as described above substituted with a cyclo(C₃-C₈)alkyl group as described above, forming such groups as for example cyclopropylmethyl, cyclohexylmethyl, cyclopentylethyl or cyclohexenylethyl. Therefore, the term (C₁-C₄)alkyl also comprises a cyclopropylmethyl group.

The term “methylene” refers to —CH₂— and may be optionally substituted.

The term “halogenated alkyl”, preferably “halogenated (C₁-C₄)alkyl” refers to an alkyl moiety (preferably (C₁-C₄)alkyl, most preferred methyl) as defined above, which is substituted either partially or in full with halogens, generally with chlorine and/or fluorine. Preferred examples of such substituents are trifluoromethyl, dichloromethyl, pentafluoroethyl, dichloropropyl, fluoromethyl and difluoromethyl.

The terms “aryl” or “Ar” refer to an aromatic carbocyclic group comprising 6 to 14, more preferably 6 to 10, carbon atoms and having at least one aromatic ring or multiple condensed rings in which at least one ring is aromatic. Preferably, aryl is phenyl, naphthyl, indanyl, indenyl, or 1,2,3,4-tetrahydro-naphthalen-1-yl; most preferred aryl is phenyl.

In addition to the substituents explicitly exemplified here within, the aryl may be substituted by two groups which are attached to adjacent carbon atoms and are combined into a saturated or partly unsaturated cyclic 5, 6, 7, or 8 membered ring system, optionally containing 1, 2 or 3 heteroatoms selected from N, O or S, the number of N atoms being 0, 1, 2 or 3 and the number of O and S atoms each being 0, 1 or 2. Preferably, the two groups which are attached to adjacent carbon atoms, are combined into a saturated cyclic 5 or 6 membered ring system, optionally containing 1, 2 or 3 heteroatoms selected from N and O, the number of N atoms being 0, 1, 2 or 3 and the number of O atoms each being 0, 1 or 2. This cyclic ring system may optionally be further substituted by one or two oxo groups and/or one or two (C₁-C₄)alkyl groups. Preferred examples of such a aryl groups are benzo[1,3]dioxolyl, 4H-chromenyl, 1,3-dihydro-isobenzofuryl, 3,4-dihydro-2H-benzooxazinyl, 3,4-dihydro-2H-benzothiazinyl, 2,3-dihydro-benzodioxinyl and 1,3-dihydro-benzoimidazolyl; in particular benzo[1,3]dioxol-5-yl, 4H-chromen-7-yl, 1,3-dihydro-isobenzofuran-5-yl, 3,4-dihydro-2H-benzo[1,4]oxazin-6-yl, 3,4-dihydro-2H-benzo[1,4]thiazin-6-yl or 2,3-dihydro-benzo[1,4]dioxin-6-yl, or substituted variants thereof, e.g. 2-methyl-4-oxo-4H-chromen-7-yl, 3-oxo-1,3-dihydro-isobenzofuran-5-yl, 4-methyl-3-oxo-3,4-dihydro-2H-benzo[1,4]oxazin-6-yl or 2-methyl-3-oxo-3,4-dihydro-2H-benzo[1,4]thiazin-6-yl.

The term “aryl-(C₁-C₄)alkyl” refers to an (C₁-C₄)alkyl group substituted with an aryl group, wherein the aryl is phenyl, naphthyl, indanyl, indenyl, or 1,2,3,4-tetrahydro-naphthalen-1-yl, preferably aryl is phenyl or naphthyl, forming such groups as for example benzyl, phenethyl, phenylpropyl, phenylbutyl, naphthylmethyl or naphthylethyl. The alkyl chain may be partially unsaturated, such as a vinyl group. The aryl moiety may optionally be substituted as defined herein.

The term “heteroaryl” refers to an aromatic carbocyclic group of having a single 4 to 8 membered ring or multiple condensed rings comprising 6 to 14, more preferably 6 to 10, ring atoms and containing at least one heteroatom selected from N, O and S, within at least one ring, the number of N atoms being 0, 1, 2 or 3 and the number of O and S atoms each being 0, 1 or 2; in which group at least one heterocyclic ring is aromatic. Examples of such groups include pyrrolyl, thienyl, furyl, pyrazolyl, imidazolyl, triazolyl, tetrazolyl, thiazolyl, isothiazolyl, oxazolyl, isoxazolyl, pyridinyl, pyrimidinyl, pyrazinyl, pyridazinyl, indolyl, indazolyl, quinolinyl, isoquinolinyl, benzofuryl, benzothiazolyl, benzotriazolyl, benzoimidazolyl, benzooxazinyl, benzothiazinyl, benzothienyl (optionally substituted with two oxo groups at the S atom) and the like. Preferably, heteroaryl is indazolyl, indolyl, isooxazolyl, pyrazolyl, thienyl, imidazolyl, pyridinyl, quinolinyl, thiazolyl, pyrrolyl, triazolyl or tetrazolyl, in particular 1H-indazol-6-yl, 1H-indol-3-yl, 1H-indol-4-yl, 1H-indol-5-yl, 1H-indol-6-yl, 1H-pyrazol-3-yl, imidazol-1-yl, isoxazol-3-yl, pyridin-2-yl, pyridin-2-yl, pyridin-3-yl, pyridin-4-yl, quinolin-3-yl, quinolin-6-yl, thiazol-2-yl, thiazol-4-yl, thiophen-2-yl, pyrrol-1-yl, 4H-[1,2,4]triazol-3-yl, tetrazol-1-yl and 1H-benzotriazol-5-yl. Most preferred heteroaryl refers to pyrrolyl, tetrazolyl, 1H-indazolyl, quinolinyl, 1H-indolyl and 1,1-dioxo-1H-benzo[b]thienyl, in particular pyrrol-1-yl, tetrazol-1-yl, 1H-indazol-6-yl, quinolin-3-yl, quinolin-6-yl, 1H-indol-4-yl, 1H-indol-5-yl, 1H-indol-6-yl and 1,1-dioxo-1H-1lambda*6*-benzo[b]thiophen-6-yl.

The term “heteroaryl-(C₁-C₄)alkyl” refers to an (C₁-C₄)alkyl group substituted with a heteroaryl group, wherein the heteroaryl is as defined herein, preferably heteroaryl is indolyl, thienyl, imidazolyl and pyridinyl, forming such groups as for example 1H-indolyl-ethyl, thienyl-ethyl, imidazolyl-propyl, pyridinyl-ethyl and pyridinyl-methyl, in particular 2-(1H-indol-3-yl)-ethyl, 2-thiophen-2-yl-ethyl, 3-imidazol-1-yl-propyl, 2-pyridin-2-yl-ethyl, pyridin-2-yl-methyl, pyridin-3-yl-methyl and pyridin-4-yl-methyl. The hetaryl moiety may optionally be substituted as defined herein.

The term “cycloheteroalkyl” refers to a four- to eight-membered heterocyclic ring containing at least one heteroatom, such as N, O or S, the number of N atoms being 0, 1 2 or 3 and the number of O and S atoms each being 0, 1 or 2, which system may be saturated, partly unsaturated or hydroaromatic, and which ring can be part of a multiple condensed ring-system in which some rings may be aromatic. Examples of such cycloheteroalkyl groups include pyrrolidinyl, tetrahydrofuryl, tetrahydrothienyl, tetrahydropyridinyl, azetidinyl, thiazolidinyl, oxazolidinyl, piperidinyl, morpholinyl, thiomorpholinyl, piperazinyl, azepanyl, diazepanyl, oxazepanyl, thiazepanyl, dihydro-1H-pyrrolyl, dihydro-1H-pyrazolyl, 3,6-dihydro-2H-pyridinyl, 1,3-dihydro-benzoimidazolyl and the like. Preferred examples of such cycloheteroalkyl groups are piperidinyl, morpholinyl, tetrahydrofuryl and 2,3-dihydro-1H-pyrazolyl, in particular piperidin-4-yl, 2,3-dihydro-1H-pyrazol-4-yl, tetrahydro-furan-3-yl and morpholin-4-yl.

The cycloheteroalkyl group may optionally be substituted by 1, 2, 3 or 4 substituents, independently selected from the group consisting of oxo, (C₁-C₄)alkyl, optionally substituted aryl or aryl-(C₁-C₄)-alkyl as defined herein. The substituents of the cycloheteroalkyl groups may be attached to any carbon, nitrogen and/or sulphur atom of the cycloheteroalkyl moiety. Substituted cycloheteroalkyl is preferably substituted with oxo, (C₁-C₄)alkyl, preferably methyl and phenyl. Preferred examples of such substituted cycloheteroalkyl groups are 1-methyl-piperidin-4-yl, 1,5-dimethyl-3-oxo-2-phenyl-2,3-dihydro-1H-pyrazol-4-yl, 2-oxo-tetrahydro-furan-3-yl and 2-oxo-pyrrolidin-1-yl.

The term “cycloheteroalkyl-(C₁-C₄)alkyl” refers to an (C₁-C₄)alkyl group substituted with a cycloheteroalkyl group, wherein the cycloheteroalkyl group is as defined herein, preferably cycloheteroalkyl is piperidinyl, pyrrolidinyl or morpholinyl, forming such groups as for example morpholinyl-ethyl, pyrrolidinyl-propyl and piperidinyl-methyl, in particular 2-morpholin-4-yl-ethyl.

When two side chains (e.g. R5 and R6 or R⁷ and R⁸) are found on a single N, e.g. when A represents —CO-NR6-, —CO-NR4-NR6- or —CO—NH—SO₂—NR6- forming such substituents as —CO-NR5R6-, —CO-NR4-NR5R6- or —CO—NH—SO₂-NR5R6-, or within the substituents —CO—NR⁷R⁸, —SO₂—NR⁷R⁸ and —NR⁷R⁸, they can be combined, including the N to which they are attached, into a heterocyclic ring of 5-, 6-, 7- or 8 atoms, which can be saturated, partly unsaturated or aromatic, and which optionally contains 1, 2 or 3 additional heteroatoms selected from N, O or S, the number of additional N atoms being 0, 1, 2 or 3 and the number of O and S atoms each being 0, 1 or 2; and which ring can be part of a multiple condensed ring-system, in which some rings may be aromatic. In one embodiment, in particular for —CO—NR⁷R⁸, —SO₂—NR⁷R⁸ and —NR⁷R⁸, the side chains form together with the N atom, to which they are attached, a heterocyclic 5-, 6-, 7- or 8-membered ring system, which is saturated, partly unsaturated or aromatic, and which optionally contains 1 or 2 additional heteroatoms selected from N, O and S, the number of additional N atoms being 0, 1 or 2 and the number of O and S atoms each being 0 or 1. In another embodiment, in particular for —CO—NR5R6-, the side chains form together with the N atom, to which they are attached, a heterocyclic 5- or 6-membered saturated ring system; which optionally contains 1 or 2 additional heteroatoms selected from N, O and S, the number of additional N atoms being 0, 1 or 2, and the number of O and S atoms each being 0 or 1. Preferred examples of such heterocyclic ring systems, including the N to which the respective side chains are attached, include:

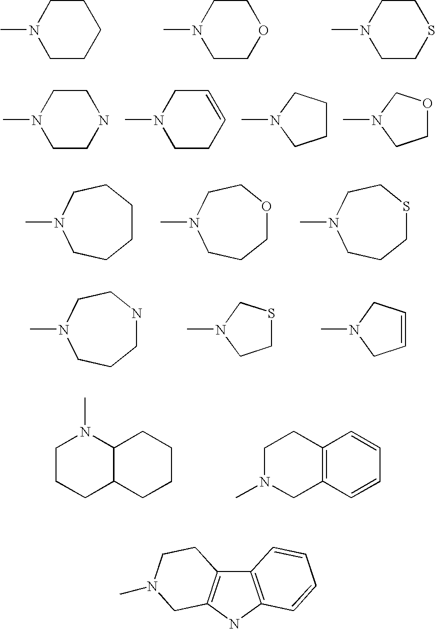

The aforementioned heterocyclic ring system can be optionally substituted by 1 or 2 substituents, which can be attached to any carbon or nitrogen atom of the heterocyclic ring system. Preferred examples of substituted heterocyclic ring systems include:

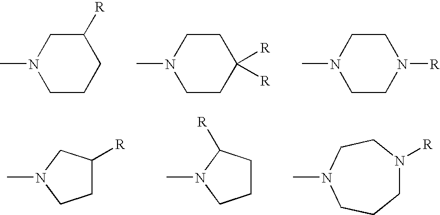

The optional 1 or 2 independently selected substituents for the heterocyclic ring system may be chosen among —(C₁-C₄)alkyl, halogenated —(C₁-C₄)alkyl, halogen, aryl, aryl-(C₁-C₄)alkyl- and heteroaryl. Preferably, the heterocyclic ring system is optionally substituted with an aryl group, the aryl group being optionally substituted with one or two substituents independently selected from —O—R, halogen, —(C₁-C₄)alkyl and halogenated —(C₁-C₄)alkyl.

### Compound Numbering (Nomenclature)

Furthermore, in an effort to maintain consistency in the naming of compounds of similar structure but differing substituents, the compounds described herein are named according to the following general guidelines. The numbering system for the location of substituents on such compounds is also provided.

The C-atoms of the steroidal core of the pregnane derivate are numbered according to the following general scheme:

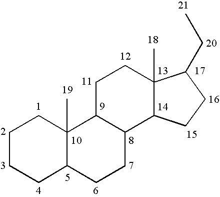

Dydrogesterone—9β,10α-Pregna-4,6-diene-3,20-dione—has the following formula:

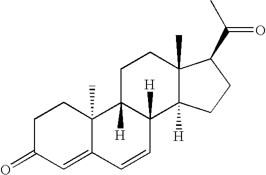

Retroprogesterone—9β,10α-Pregna-4-ene-3,20-dione—has the following formula:

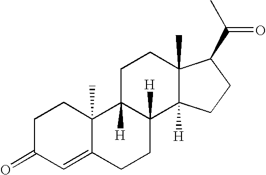

General structural formulas are typically designated with a number in Roman format I, II, III etc. Intermediates are indicated with the same numbers in Roman format as of the corresponding general formulas and a further letter or number, e.g. X-H for a particular derivative falling under the scope of general formula X. The compounds of the invention are designated No. 1, No. 2 etc.

In order to simplify the naming of the compounds the following approach was used: for example, the name of the known compound “11β-Acetoxy-9β,10α-pregna-4,6-diene-3-20-dione” corresponding to “Acetic acid 17-acetyl-10,13-dimethyl-3-oxo-2,3,8,9,10,11,12,13,14,15,16,17-dodecahydro-1H-9β,10α-cyclopenta[a]phenanthren-11β-yl ester” in IUPAC nomenclature was simplified by using the corresponding steroid name “Acetic acid dydrogesterone-11β-yl ester”, since the compounds of the present invention all represent ester derivatives of dydrogesterone carrying the ester group in C11 position and optionally further substituents in C2, C3 and C17 position.

### EMBODIMENTS

### Subclaims and Further Embodiments

The invention relates to compounds of general formula (I) or (Ib)

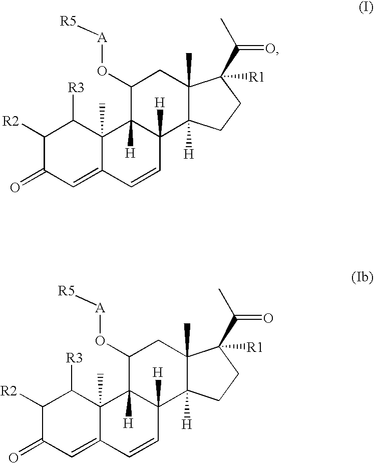

wherein A represents —CO—, —CO-NR6-, —CO—NH-NR6- or —CO—NH—SO₂-NR6-, wherein all residues R1 to R14 are as defined above. Particularly, A should represent —CO— or —CO-NR6-, in particular —CO-NR6-. Preferably A only represents —CO— under the proviso that R1 is selected from —O—(C₁-C₄)alkyl and —O—CO—(C₁-C₄)alkyl. Alternatively, A only represents —CO— under the proviso that R2 and R3 together form a methylene group.

In one embodiment, the compounds of general formula (I) or (Ib) are characterized that R1 is selected from hydrogen, —OH, —O—(C₁-C₄)alkyl and —O—CO—(C₁-C₄)alkyl; in particular R1 is selected from hydrogen and —O—(C₁-C₄)alkyl, e.g. ethoxy.

Furthermore, the invention relates to compounds of general formula (I) or (Ib), wherein R2 and R3 are both hydrogen or wherein R2 and R3 together form a methylene group.

Another embodiment of the invention relates to compounds of general formula (I) or (Ib), wherein A represents —CO— or —CO-NR6-; and R1 is selected from hydrogen and —O—(C₁-C₄)alkyl. In particular, A represents —CO-NR6- and R1 is selected from hydrogen and —O—(C₁-C₄)alkyl.

A further aspect of the invention relates to compounds of general formula (I) or (Ib), wherein A represents —CO—; and R1 is selected from —O—(C₁-C₄)alkyl and —O—CO—(C₁-C₄)alkyl. In particular, A represents —CO— and R1 represents —O—(C₁-C₄)alkyl.

According to another embodiment, the invention comprises compounds of general formula (I) or (Ib), wherein A represents —CO—; R1 is selected from hydrogen, —OH, —O—(C₁-C₄)alkyl and —O—CO—(C₁-C₄)alkyl; and R2 and R3 together form a methylene group. In particular, A represents —CO—; R1 is selected from hydrogen and —O—(C₁-C₄)alkyl; and R2 and R3 together form a methylene group.

In another embodiment, the substituent R5 of the compounds of formula (I), (Ib), (II), (IIb), (III), (IIIb), (IV) or (IVb) is selected from —(C₁-C₆)alkyl, aryl, aryl-(C₁-C₄)alkyl, heteroaryl, heteroaryl-(C₁-C₄)alkyl, cycloheteroalkyl and cycloheteroalkyl-(C₁-C₄)alkyl, whereby all residues can be optionally substituted as defined herein. Preferably, R5 is selected from optionally substituted —(C₁-C₆)alkyl, aryl, aryl-(C₁-C₄)alkyl, heteroaryl, heteroaryl-(C₁-C₄)alkyl, cycloheteroalkyl and cycloheteroalkyl-(C₁-C₄)alkyl. In particular, R5 is selected from optionally substituted aryl, aryl-(C₁-C₄)alkyl, heteroaryl, heteroaryl-(C₁-C₄)alkyl, cycloheteroalkyl and cycloheteroalkyl-(C₁-C₄)alkyl as defined herein.

The substituent R5 of compounds of general formula (I) should preferably only represent unsubstituted or even substituted alkyl, i.e. —(C₁-C₈)alkyl, —(C₁-C₆)alkyl or —(C₁-C₄)alkyl, as defined herewithin, under the proviso that A represents —CO-NR6-, —CO-NR4-NR6- or —CO—NH—SO₂-NR6-. Preferably R5 should not represent unsubstituted or even substituted —(C₁-C₈)alkyl, —(C₁-C₆)alkyl or —(C₁-C₄)alkyl, if A represents —CO—; i.e. R5 should preferably not represent unsubstituted or even substituted —(C₁-C₈)alkyl, —(C₁-C₆)alkyl or —(C₁-C₄)alkyl in compounds of general formula (II) or (IIb).

The substituent R5 of compounds of general formula (I) should preferably only represent unsubstituted aryl, in particular unsubstituted phenyl, under the proviso that R1 represents —O—(C₁-C₄)alkyl or —O—CO—(C₁-C₄)alkyl. Preferably R5 should not represent unsubstituted aryl or unsubstituted phenyl, if R1 represents hydrogen or (C₁-C₄)alkyl.

When R5 represents —(C₁-C₆)alkyl, this (C₁-C₆)alkyl group is optionally substituted with one or two substituents independently selected from the group consisting of —O—R⁹ and —CN.

When R5 represents aryl or aryl-(C₁-C₄)alkyl, the aryl moiety of the aryl or aryl-(C₁-C₄)alkyl group is optionally substituted with one or more substituents independently selected from —O—R9, —S—R⁹, —O—CO—NHR¹⁰, —CO—R¹¹, —SO₂—R¹¹, —CO—NR⁷R⁸, —SO₂—NR⁷R⁸, —CN, —CH═N—O—R¹², —CH═N—O—CO—NHR¹⁰, —NR⁷R⁸, —NR¹³—CO—R¹¹, —NR¹³—SO₂—R¹¹, halogen, —(C₁-C₄)alkyl, halogenated —(C₁-C₄)alkyl, cycloheteroalkyl and heteroaryl, the number of said substituents being 1, 2, 3, 4 or 5 for halogen, and 1, 2 or 3 for any combination of said substituents, and wherein the cycloheteroalkyl or heteroaryl is optionally substituted with oxo or —(C₁-C₄)alkyl. Alternatively, the aryl moiety of the aryl or aryl-(C₁-C₄)alkyl group representing R5 is optionally substituted by two groups which are attached to adjacent carbon atoms and are combined into a saturated or partly unsaturated cyclic 5- or 6-membered ring system, optionally containing 1, 2 or 3 heteroatoms selected from N, O and S, the number of N atoms being 0, 1, 2 or 3 and the number of O and S atoms each being 0, 1 or 2; whereby the cyclic ring system is optionally substituted by one or two substituents independently selected from oxo and —(C₁-C₄)alkyl.

When R5 represents heteroaryl or heteroaryl-(C₁-C₄)alkyl, the heteroaryl moiety of the heteroaryl or heteroaryl-(C₁-C₄)alkyl group is optionally substituted with a —(C₁-C₄)alkyl-CO—O—R⁹, —(C₁-C₄)alkyl or aryl group.

When R5 represents cycloheteroalkyl or cycloheteroalkyl-(C₁-C₄)alkyl, the cycloheteroalkyl moiety of the cycloheteroalkyl or cycloheteroalkyl-(C₁-C₄)alkyl group is optionally substituted with 1, 2, 3 or 4 substituents independently selected from the group consisting of oxo, —(C₁-C₄)alkyl and aryl.

When R5 represents aryl-(C₁-C₄)alkyl, heteroaryl-(C₁-C₄)alkyl or cycloheteroalkyl-(C₁-C₄)alkyl, the —(C₁-C₄)alkyl moiety of the aryl-(C₁-C₄)alkyl, heteroaryl-(C₁-C₄)alkyl or cycloheteroalkyl-(C₁-C₄)alkyl group is optionally substituted with an oxo group.

In one embodiment, the residue A in compounds of formula (I), (Ib), (II), (IIb), (III), (IIIb), (IV) or (IVb) represents —CO-NR6-, —CO—NH-NR6- or —CO—NH—SO₂-NR6-, and then the substituent R6 is selected from hydrogen and —(C₁-C₄)alkyl, optionally substituted with an —O—R⁹ group. Alternatively, R5 and R6 form together with the nitrogen atom, where R5 and R6 are attached, a heterocyclic 5- or 6-membered saturated ring system; which optionally contains 1 or 2 additional heteroatoms selected from N, O and S, the number of additional N atoms being 0, 1 or 2, and the number of O and S atoms each being 0 or 1; and which ring system is optionally substituted with an aryl group optionally substituted with —O—R¹⁴ or halogenated —(C₁-C₄)alkyl.

In one embodiment, the residues R⁷, R⁸, R⁹, R¹⁰, R¹¹, R¹², R¹³ and R¹⁴ mentioned above within the definitions given for R5 and R6, are independently selected from the group consisting of hydrogen, —(C₁-C₄)alkyl, and halogenated —(C₁-C₄)alkyl.

Representative PR modulator compounds (i.e., agonists, partial agonists and antagonists) according to the present invention include
(1,1-Dioxo-1H-1lambda*6*-benzo[b]thiophen-6-yl)-carbamic acid dydrogesterone-11β-yl ester
(1H-Indazol-6-yl)-carbamic acid dydrogesterone-11β-yl ester
(1H-Indol-5-yl)-carbamic acid dydrogesterone-11β-yl ester
(1-Methyl-1H-indazol-6-yl)-carbamic acid dydrogesterone-11β-yl ester
(2,3-Dihydro-benzo[1,4]dioxin-6-yl)-carbamic acid dydrogesterone-11β-yl ester
(2-Methoxy-phenyl)-carbamic acid dydrogesterone-11β-yl ester
(2-Methyl-3-oxo-3,4-dihydro-2H-benzo[1,4]thiazin-6-yl)-carbamic acid dydrogesterone-11β-yl ester
(3,4,5-Trimethoxy-phenyl)-carbamic acid dydrogesterone-11β-yl ester
(3-Acetylamino-phenyl)-carbamic acid dydrogesterone-11β-yl ester
(3-Acetyl-phenyl)-carbamic acid dydrogesterone-11β-yl ester
(3-Bromo-4-methyl-phenyl)-carbamic acid dydrogesterone-11β-yl ester
(3-Carbamoyl-phenyl)-carbamic acid dydrogesterone-11β-yl ester
(3-Cyano-4-fluoro-phenyl)-carbamic acid dydrogesterone-11β-yl ester
(3-Cyano-phenyl)-carbamic acid dydrogesterone-11β-yl ester
(3-Methoxy-5-tetrazol-1-yl-phenyl)-carbamic acid dydrogesterone-11β-yl ester
(3-Methoxy-phenyl)-carbamic acid 17α-ethoxy-1,2-methylen-dydrogesterone-11β-yl ester
(3-Methoxy-phenyl)-carbamic acid 17α-ethoxy-dydrogesterone-11β-yl ester
(3-Methoxy-phenyl)-carbamic acid dydrogesterone-11β-yl ester
(3-Methylsulfanyl-phenyl)-carbamic acid dydrogesterone-11β-yl ester
(3-Pyrrol-1-yl-phenyl)-carbamic acid dydrogesterone-11β-yl ester
(3-Trifluoromethoxy-phenyl)-carbamic acid dydrogesterone-11β-yl ester
(4-Acetylamino-phenyl)-carbamic acid dydrogesterone-11β-yl ester
(4-Difluoromethoxy-phenyl)-carbamic acid dydrogesterone-11β-yl ester
(4-Methoxy-benzyl)-carbamic acid dydrogesterone-11β-yl ester
(4-Methoxy-phenyl)-carbamic acid dydrogesterone-11β-yl ester
(4-Methyl-3-oxo-3,4-dihydro-2H-benzo[1,4]oxazin-6-yl)-carbamic acid dydrogesterone-11β-yl ester
(4-Methylsulfanyl-phenyl)-carbamic acid dydrogesterone-11β-yl ester
(4-Morpholin-4-yl-phenyl)-carbamic acid dydrogesterone-11β-yl ester
(4-Trifluoromethoxy-phenyl)-carbamic acid dydrogesterone-11β-yl ester
[3-(2-Oxo-pyrrolidin-1-yl)-phenyl]-carbamic acid dydrogesterone-11β-yl ester
4-(2-Methoxy-phenyl)-piperazine-1-carboxylic acid dydrogesterone-11β-yl ester
4-(3-Trifluoromethyl-phenyl)-piperazine-1-carboxylic acid dydrogesterone-11β-yl ester
4-Methoxy-benzoic acid dydrogesterone-11β-yl ester
Benzo[1,3]dioxol-5-yl-carbamic acid 17-ethoxy-dydrogesterone-11β-yl ester
Benzo[1,3]dioxol-5-yl-carbamic acid dydrogesterone-11β-yl ester
Benzo[1,3]dioxol-5-ylmethyl-carbamic acid dydrogesterone-11β-yl ester
Benzoic acid 17α-ethoxy-dydrogesterone-11β-yl ester
Benzoic acid dydrogesterone-11β-yl ester
Isopropyl-carbamic acid dydrogesterone-11β-yl ester
Quinolin-3-yl-carbamic acid dydrogesterone-11β-yl ester
Quinolin-6-yl-carbamic acid dydrogesterone-11β-yl ester
   Administration forms (Pharmaceutical Formulations)

The method of the invention is primarily intended for treatment in a mammal, preferably in humans and other primates, of diseases, disorders or conditions mediated by progesterone receptors, or of diseases, disorders or conditions that can be treated via modulation of those receptors. In particular, the invention concerns the therapeutic use of said novel retrosteroidal derivatives in the treatment or prevention of benign gynecological disorders, especially endometriosis and uterine fibroids, in hormonal female contraception or in hormone replacement therapy

The compounds may be administered orally, dermally, parenterally, by injection, by pulmonal or nasal delivery, or sublingually, or by topical administration, i.e. rectally, vaginally, or within the intrauterine cavity, in dosage unit formulations. The term “administered by injection” includes intravenous, intraarticular, intramuscular (e.g. by depot injection where the active compounds are released slowly into the blood from the depot and carried from there to the target organs), intraperitoneal, intradermal, subcutaneous, and intrathecal injections, as well as use of infusion techniques. Dermal administration may include topical application or transdermal administration. One or more compounds may be present in association with one or more non-toxic pharmaceutically acceptable auxiliaries such as excipients, adjuvants (e.g. buffers), carriers, inert solid diluents, suspensing agents, preservatives, fillers, stabilizers, anti-oxidants, food additives, bioavailability enhancers, coating materials, granulating and disintegrating agents, binding agents etc., and, if desired, other active ingredients.

The pharmaceutical composition may be formulated for example as immediate release, sustained release, pulsatile release, two or more step release, depot or other kind of release formulations.

The manufacture of the pharmaceutical compositions according to the invention may be performed according to methods known in the art and will be explained in further detail below. Commonly known and used pharmaceutically acceptable auxiliaries as well as further suitable diluents, flavorings, sweetening agents, coloring agents etc. may be used, depending on the intended mode of administration as well as particular characteristics of the active compound to be used, such as solubility, bioavailability etc. Suitable auxiliaries and further ingredients may be such as recommended for pharmacy, cosmetics and related fields and which preferably are listed in the European Pharmacopoeia, FDA approved or cited in the “GRAS” list (FDA List of food additives that are ‘generally recognized as safe’ (GRAS)).

One mode of administration of the compounds of general formula (I) or of pharmaceutical compositions comprising one or more of said compounds is oral administration, e.g., by tablets, pills, dragees, hard and soft gel capsules, granules, pellets, aqueous, lipid, oily or other solutions, emulsions such as oil-in-water emulsions, liposomes, aqueous or oily suspensions, syrups, elixiers, solid emulsions, solid dispersions or dispersible powders. For the preparation of pharmaceutical compositions for oral administration, the compounds suitable for the purposes of the present invention as defined above can be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire™). In the pharmaceutical composition, the active ingredients may also be dispersed in a microparticle, e.g. a nanoparticulate, composition.

For parenteral administration, the active agents can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration the active agent can be in the form of an aqueous, lipid, oily or other kind of solution or suspension or even administered in the form of liposomes or nano-suspensions.

Transdermal administration can be accomplished by suitable patches, as generally known in the art, specifically designed for the transdermal delivery of active agents, optionally in the presence of specific permeability enhancers. Furthermore, also emulsions, ointments, pastes, creams or gels may be used for transdermal delivery.

Another suitable mode of administration is via intravaginal devices (e.g. vaginal rings) or intrauterine systems (IUS) and intrauterine devices (IUD), respectively, containing reservoirs for controlled release of active agents over extended periods of time. Such IUS or IUDs (as, e.g., MIRENA™) is introduced into the uterine cavity where it continuously releases defined amounts of hormone for up to 5 years (or until the system is removed).

For rectal or vaginal administration of the drug the compounds may also be administered in the form of suppositories. These compositions can be prepared by mixing the drug with a suitable non-irritating excipient which is solid at ordinary temperatures but liquid at the rectal or vaginal temperature and will therefore melt in the rectum or vagina to release the drug.

A further drug formulation is a formulation intended for the topical, local and/or regional administration of the compound to the reproductive organs, in particular to a body region selected from the group consisting of the uterus, fallopian tubes, peritoneal space, pelvic cul-de-sac, ovaries, and urinogenital tract, in amounts effective to treat various conditions, particularly local diseases of the female reproductive system, such as pelvic, uterine, cervical and vaginal diseases, as described e.g. within EP 0977555 A1^{xxxix}, U.S. Pat. No. 5,993,856^{xi}, U.S. Pat. No. 6,652,874ₓᵢᵢ, or U.S. Pat. No. 6,416,778^{xiii}. The formulation comprises drug particles, preferably in the form of a micro- or nano-particles, suitable for regional administration of an effective amount of drug, wherein the effective amount is a dosage which results in low serum drug levels and reduced side effects as compared to systemic administration of the drug. In particular, the formulation comprises a carrier promoting quick uptake of the drug into the blood stream, a carrier manipulating release of drug, or a carrier promoting adhesion of the drug selected from the group consisting of a liquid suspension or dispersion, a hydrogel suspension or dispersion, a topical ointment, a cream, a lotion, and a foam.

Another mode of administration is by implantation of a depot implant comprising an inert carrier material, such as biologically degradable polymers or synthetic silicones such as e.g. silicone rubber. Such implants are designed to release the active agent in a controlled manner over an extended period of time (e.g., 3 to 5 years).

It will be appreciated by those skilled in the art that the particular method of administration will depend on a variety of factors, all of which are considered routinely when administering therapeutics. It will also be understood, however, that the actual dosages of the agents of this invention for any given patient will depend upon a variety of factors, including, but not limited to the activity of the specific compound employed, the particular composition formulated, the mode of administration, time of administration, route of administration and the particular site, host, and disease being treated, and furthermore the age of the patient, the body weight of the patient, the general health of the patient, the gender of the patient, the diet of the patient, rate of excretion, drug combinations, and the severity of the condition undergoing therapy. It will be further appreciated by one skilled in the art that the optimal course of treatment, i.e., the mode of treatment and the daily number of doses of a compound of Formula I or a pharmaceutically acceptable salt thereof given for a defined number of days, can be ascertained by those skilled in the art using conventional treatment tests. Optimal dosages for a given set of conditions may be ascertained by those skilled in the art using conventional dosage-determination tests in view of the experimental data for a given compound. For oral administration, an exemplary daily dose generally employed will be from about 0.001 μg/kg to about 10 mg/kg of total body weight, whereby courses of treatment may be repeated at appropriate time intervals. Administration of pro-drugs may be dosed at weight levels that are chemically equivalent to the weight levels of the fully active compounds. The daily dosage for parenteral administration will generally be from about 0.001 μg/kg to about 10 mg/kg of total body weight. A daily rectal dosage regimen will generally be from about 0.001 μg/kg to about 20 mg/kg of total body weight. A daily vaginal dosage regimen will generally be from about 0.001 μg/kg to about 10 mg/kg of total body weight. The daily topical dosage regimen will generally be from about 0.01 μg to about 10 mg administered between one to four times daily. The transdermal concentration will generally be that required to maintain a daily dose of from 0.001 μg/kg to 10 mg/kg of total body weight. The total dosage of administration forms releasing the drug compound over a prolonged period of time, i.e. from about several weeks to some years, depends on the time of administration, on the kind of device (intravaginal devices, intrauterine systems, intrauterine devices, implants etc.) and on the kind of release behaviour of the particular device. In general, the daily released dose of active compound will be from about 0.001 μg/kg to about 1 mg/kg of total body weight. Since the devices often only need to achieve a certain local and/or regional concentration of active compound, the daily released dosage can be lower in comparison to e.g. oral administration.

### Abbreviations and Acronyms

As employed herein, the following terms have the indicated meanings.

ACN acetonitrile

AP alkaline phosphatase

aq aqueous

AR androgen receptor

Bn benzyl

Bu butyl

cat catalytic amount

CH cyclohexane

conc. concentrated

d day(s)

DCC Dicyclohexylcarbodiimide

DCM dichloromethane=CH₂Cl₂

DDQ 2,3-dichloro-5,6-dicyano-p-benzoquinone

DEE Diethyl ether

DIBAH diisobutyl-aluminium-hydride

DIPEA N,N-diisopropylethylamine

DMAP 4-(N,N-dimethylamino)-pyridine

DME dimethyl ethylene glycol=1,2-dimethoxyethane

DMF N,N-dimethylformamide

DMSO dimethylsulfoxide

E1 estron

E2 estradiol

EDCl 1-(3-dimethylaminopropyl)-3-ethylcarbodiimide

EDCl.HCl 1-(3-dimethylaminopropyl)-3-ethylcarbodiimide hydrochloride

ER estrogen receptor

EtOAc ethyl acetate

GR glucocorticoid receptor

GRAS ‘generally recognized as safe’

h hour(s)

HMPA hexamethylphosphoramide

HOBT 1-Hydroxybenzotriazole Hydrate

HPLC High Performance Liquid Chromatography

HSD hydroxysteroid dehydrogenase

Hünig base N-Ethyldiisopropylamine=N(iPr)2Et=EDIPA):

IUD intrauterine device

LAH lithium aluminium hydride

MeOH methanol

min minute(s)

MOM methoxy methyl

m.p. melting point

MTBE methyl tertiary butyl ether

NAD(P)[H] nicotinamide-adenine-dinucleotide (phosphate) [reduced NAD(P)]

NMM N-methylmorpholine

NMMO N-methylmorpholine-N-oxide

NMR nuclear magnetic resonance

PCC pyridinium chlorochromate

PG protection group

PR progesterone receptor

prep preparative

pTosOH para-toluene sulphonic acid

pTosCl para-toluene sulphonic acid chloride

Rt Retention time

RT room temperature

sat saturated

SPRM selective progesterone receptor modulator

STS steroid sulphatase

TEA triethylamine

TEMPO 2,2,6,6-tetramethyl-1-piperidinyloxy, free radical

THF tetrahydrofuran

THP tetrahydropyran

TLC thin-layer chromatography

### General Preparative Methods

The compounds of the present invention may be prepared from 9β,10α-steroids by use of known chemical reactions and procedures. Nevertheless, the following general preparative methods are presented to aid the reader in synthesizing the SPRM compounds of the present invention, with specific details provided below in the experimental section to illustrate working examples. All variable groups of these methods are as described in the generic description if they are not specifically defined below.

It is recognized that compounds of the invention with each claimed optional functional group may not be prepared by each of the below-listed methods. Within the scope of each method, optional substituents may appear on reagents or intermediates which may act as protecting or otherwise non-participating groups. Utilizing methods well known to those skilled in the art, these groups are introduced and/or removed during the course of the synthetic schemes which provide the compounds of the present invention.

### Flow Diagrams

### A. Educts of General Formula (V)

A 9β,10α-steroidal (retrosteroidal) compound of general Formula (V)

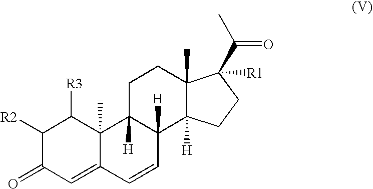

wherein
R1 is selected from hydrogen, —OH, —O—(C₁-C₄)alkyl, and —O—CO—(C₁-C₄)alkyl, and
R2 and R3 are both hydrogen or together form a methylene group,
   and which can be used as educt for the preparation of the compounds of the present invention, may be prepared from known retrosteroids by use of known chemical reactions and procedures. Nevertheless, the following general preparative methods are presented to aid the reader in synthesizing the educts used in the present invention. All variable groups of these methods are as described in the generic description if they are not specifically defined below.

The sequence of steps for the general schemes to synthesize the compounds of the present invention is shown below. In each of the Schemes the R groups (e.g., R1, R2, etc.) correspond to the specific substitution patterns noted in the Description and the Examples.

A-I. Introduction of the 1,2-methylene Group

The following Scheme I shows the optional reaction that commercially available Dydrogesterone of formula (V-1) is substituted in the 1,2 position with a methylene group.

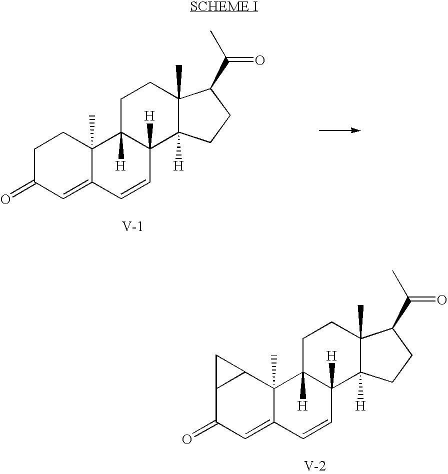

The introduction of the 1,2-methylene group might be performed according to the well known procedures as described by Halkes et al [1972]^{xxxiii} and within U.S. Pat. No. 3,937,700^{xxvii} for 17α-Hydroxy-9β,10α-pregna-4,6-diene-3,20-dione by dehydrogenation and subsequent reaction with dimethylsulfoxonium methylide. The introduction of the methylene group might also be performed at a later stage of the overall synthesis of the compounds of the invention, e.g. after introduction of the hydroxyl function in C11 and subsequent modification.

### A-II. Reduction of the Dydrogesterone to Retroprogesterone

Depending on the subsequent reaction steps, it might be necessary to convert the optionally 1,2 methylene substituted Dydrogesterone of general formula V-1,2 into the corresponding 9β,10α-pregna-4-ene-3,20-dione (9β,10α-Progesterone) of general formula V-3,4 as displayed in SCHEME II

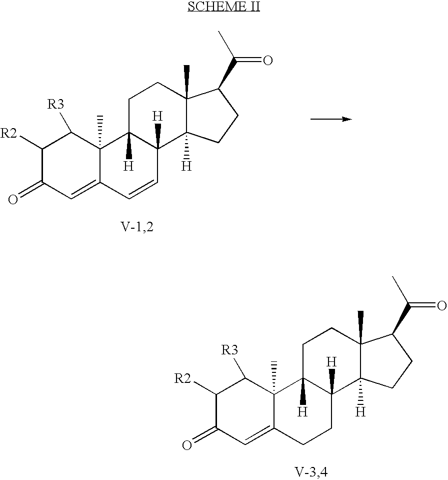

wherein R2 and R3 are both hydrogen or together form a methylene group.
   The conversion can be carried out under reducing conditions according to the procedures disclosed in U.S. Pat. No. 3,555,053^{xxx}.

### A-III. Functionalization of the C17 Position

The introduction of an additional side chain in C17α position in order to obtain compounds of general formula (V-A) or (V-B) might be achieved by methods as displayed in Scheme III and according to the procedures described by Halkes & van Moorselaar [1969]^{xliii}, within U.S. Pat. Nos. 3,555,053^{xxx} and 3,937,700^{xxvii}, and within Belgian patent No. 577,615^{xliv}. In a first step, the introduction of a 17α-hydroxy group might be carried out, followed by subsequent etherification or esterification of the hydroxyl group at carbon atom 17 as second step. If necessary to obtain compounds of general formula (V-B), the double bond in C₆-C₇ position might be finally reintroduced by dehydrogenation.

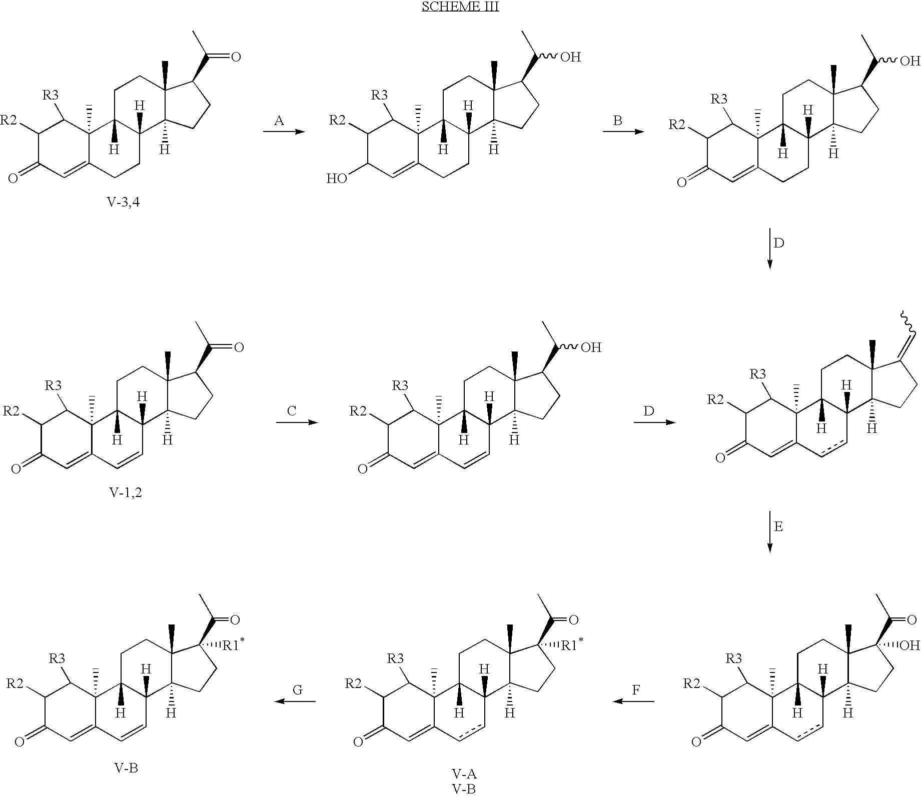

wherein R2 and R3 are both hydrogen or together form a methylene group, and
wherein R1* is —OH, —O— (C₁-C₄)alkyl, or —O—CO—(C₁-C₄)alkyl.

The functionalization of the C17 position can be achieved by starting with the introduction of a —OH group in C17 alpha position: For example, the (9β,10α)-pregna-4-ene-3,20-dione of formula V-3 or V-4 can be reduced by using a suitable reducing agent such as LAH to produce the corresponding 3,20-diol (step A). The 3-hydroxy group is then selectively re-oxidized by means of a selective oxidizing agent such as DDQ in an aromatic solvent or manganese dioxide (step B).

Alternatively, the reduction might be carried out using (9β,10α)-pregna-4,6-diene-3,20-dione of formula V-1 or V-2 as educt and a suitable selective reducing agent such as Bu₄NBH₄ directly delivering 20-hydroxy-(9β,10α)-pregna-4,6-diene-3-one (step C).

The resulting 20-hydroxy-(9β,10α)-pregna-4-ene-3-one or 20-hydroxy-(9,10α)-pregna-4,6-diene-3-one is further dehydrated by tosylation with tosyl chloride in pyridine. Subsequent treatment of the generated tosylate with a base such as pyridine or collidine affords the 17,20 unsaturated derivative in a mixture of cis and trans isomers (step D).

The latter compound is then oxygenated using a suitable oxidizing agent such as an amine oxide, e.g. NMMO, as stoichiometric oxidizing agent and additional hydrogen peroxide in the presence of a catalytic amount of osmium tetroxide to produce the corresponding 17α-hydroxy-9β,10α-pregna-4-ene-3,20-dione or 17α-hydroxy-9β,10α-pregna-4,6-diene-3,20-dione (step E).

This compound may be further modified by subjection to an etherification or esterification reaction at the hydroxyl group at the carbon atom C17 (step F), whereby the reactions are generally described within Belgian patent specification BE 577,615^{xliv} or U.S. Pat. No. 3,937,700^{xxvii}.

a) Esterification: Suitable acylating agents are carboxylic acids, carboxylic acid anhydrides or carboxylic acid chlorides in the presence of a catalyst such as p-toluene sulphonic acid, trifluoroacetic acid, anhydride or pyridine-HCl or in the presence of an acid binder such as an organic base, for example, pyridine or collidine. The acylation reaction is carried out in the presence of a solvent such as a hydrocarbon, for example, benzene or toluene. The reaction temperature may vary between room temperature and the boiling point of the solvent used. Since—if the starting material contains, apart from the 17-OH group, one or more further OH-groups—these will also be esterified, the further OH-groups have to be protected in advance.
b) Etherification: The corresponding ether derivatives might be obtained by reaction with suitable alkylating agents, e.g. by reaction with an alkylhalide in the presence of Ag₂O, or by reaction with a suitable dialkylsulfate, e.g. EtSO₄Et.

If necessary, the resulting compounds might then be again dehydrogenated to afford the 4,6 unsaturated derivative of general formula V-B (step G).

The two functionalization steps at the C2-C3 (SCHEME I) and the C17 position (SCHEME III) might also be performed in the alternative order.

B. Introduction of the hydroxy function in C11-Educts of General Formula (VI)

The introduction of any side chain in the C11 position of the retrosteroidal core affords the hydroxylation of the respective carbon atom delivering 9β,10α-steroidal (retrosteroidal) compounds of general formulae (VI) or (VI-b)

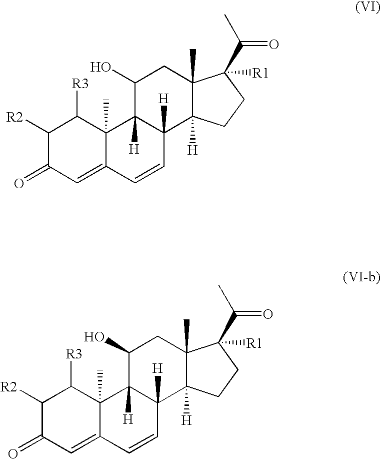

wherein
R1 is selected from hydrogen, —OH, —O—(C₁-C₄)alkyl, and —O—CO—(C₁-C₄)alkyl, and
R2 and R3 are both hydrogen or together form a methylene group.
   The hydroxylation of the respective carbon atom can be achieved starting from the corresponding intermediate compound of general formula (V) as depicted in the following general SCHEME IV.

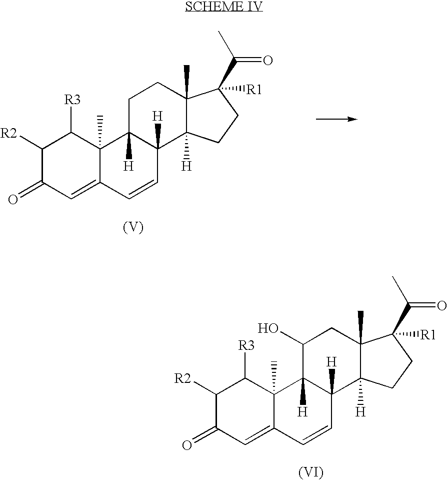

wherein R2 and R3 are both hydrogen or together form a methylene group, and
wherein R1 is hydrogen, —OH, —O— (C₁-C₄)alkyl or —O—CO—(C₁-C₄)alkyl.

This hydroxylation is typically achieved by a microbial transformation step, a process well known in the state of the art. Typically, fungal strains of the species Aspergillus or Rhizopus are used for 11α-hydroxylation of steroids with 9α,10β-conformation (as disclosed e.g. in European patent application EP 0028309^{xlv} and U.S. Pat. No. 6,046,023^{xlvi}). The C1-hydroxylation of retrosteroids, for example of 9β,10α-progesterone, in the 11α-position with the fungal strain Aspergillus ochraceus NRRL405 was described by van der Sijde et al [1966]^{xlvii}. Furthermore a publication by Saucy et al [1966]^{xlviii} discloses the microbial hydroxylation of retrosteroids in the C11 position while using Aspergillus ochraceus for 11α-hydroxylation and an unrevealed microorganism for 11β-hydroxylation. Additionally, the patent specification GB 1,111,320 disclosed the microbial hydroxylation of some specific retrosteroids in the C11β position.

An optimized process for the microbial in vitro transformation of a 9β,10α-steroidal compound of the following general formula

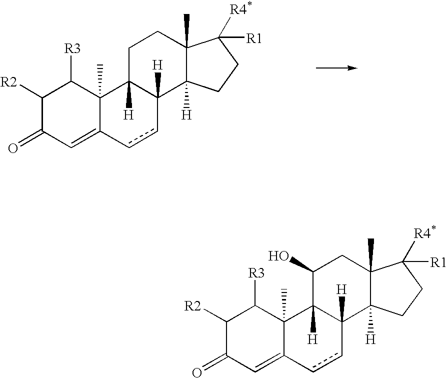

wherein
R1 and R4* together form an oxygen, OR R4* is a β-acetyl group and R1 is selected from hydrogen, —OH, —O—(C₁-C₄)alkyl, and —O—CO—(C₁-C₄)alkyl, and
R2 and R3 are both hydrogen or together form a methylene group,
   into its corresponding 11β-hydroxyl analogue by using a bacterial microorganism of the species Amycolatopsis mediterranei has been described within international patent application WO 2007082891^{xlix}. Preferably the Amycolatopsis mediterranei strain is selected from the group consisting of Amycolatopsis mediterranei LS30, DSM 43304 (corresponding to ATCC 13685, CBS 121.63, CBS 716.72, DSM 40501, IFO 13415, IMET 7651, ISP 5501, JCM 4789, KCC S-0789, LBG A 3136, NBRC 13142, NBRC 13415, NCIB 9613, NRRL B-3240, RIA 1376 or VKM Ac-798), DSM 40773, and DSM 46096 (corresponding to ATCC 21411, IMET 7669). The preferably used microorganism is bacterial strain Amycolatopsis mediterranei LS30 as deposited under DSM 17416 at the “Deutsche Sammlung von Mikroorganismen und Zellkulturen”, the DSMZ (Address: Mascheroder Weg 1b, D-38124 Braunschweig, Germany).

### C. Further Modification of the C11 Hydroxyl Group—Compounds of the Invention (I)

Starting from the 11β-hydroxyl or 11α-hydroxyl 9β,10α-steroidal compound of general formula (VI) the compounds of the invention can be synthesized by a series of several reaction steps as summarized in the following SCHEME V:

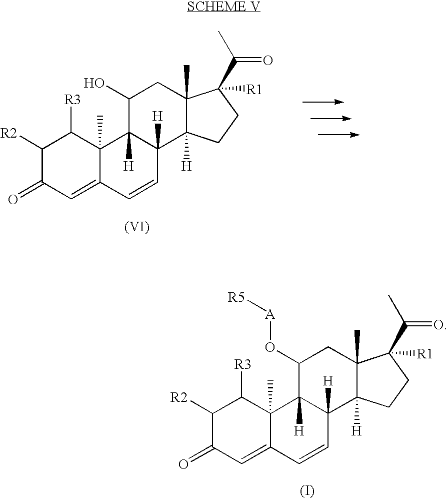

The sequence of steps for the general schemes to synthesize the compounds of the present invention is shown below. In each of the Schemes the R groups (e.g., R1, R2, etc.) correspond to the specific substitution patterns noted in the Description and the Examples unless explicitly depicted otherwise. However, it will be understood by those skilled in the art that other functionalities disclosed herein at the indicated positions of compounds of formulae I, II, III and IV also comprise potential substituents for the analogous positions on the structures within the Schemes.

### C-1. Synthesis of Compounds of Formula (I) Wherein A Represents —CO—

The compounds of general formula (VI) can be esterified to yield the corresponding 11α or 11β-(esterified hydroxyl)-9β,10α steroids by a reaction with the corresponding carboxylic acid R5-COOH as depicted in the following general SCHEME VI.

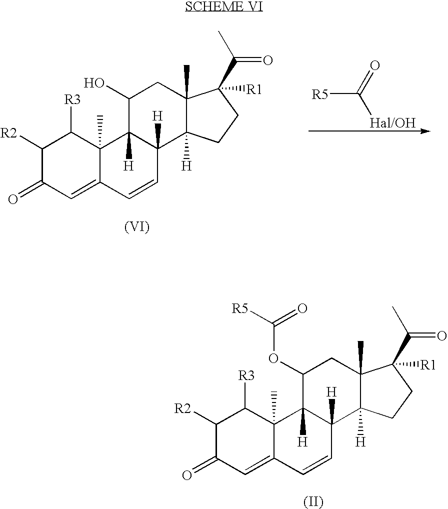

The esterification can be carried out in accordance with methods known to the skilled artisan, for example, by reaction of the 11-hydroxy-retrosteroid with a reactive derivative of a carboxylic acid (e.g. the anhydride or the halide R5-CO-Hal, preferably the chloride R5-CO—Cl) in the presence of a base such as pyridine.

C-II. Synthesis of Compounds of Formula (I) wherein A Represents —CO-NR6

The compounds of general formula (VI) can be converted into the corresponding 11α or 11β-substituted carbamate-9β,10α steroids of general formula (III) by different type of reactions depending on the nature of the substitutents R5 and R6 as depicted in the following general SCHEME VII.

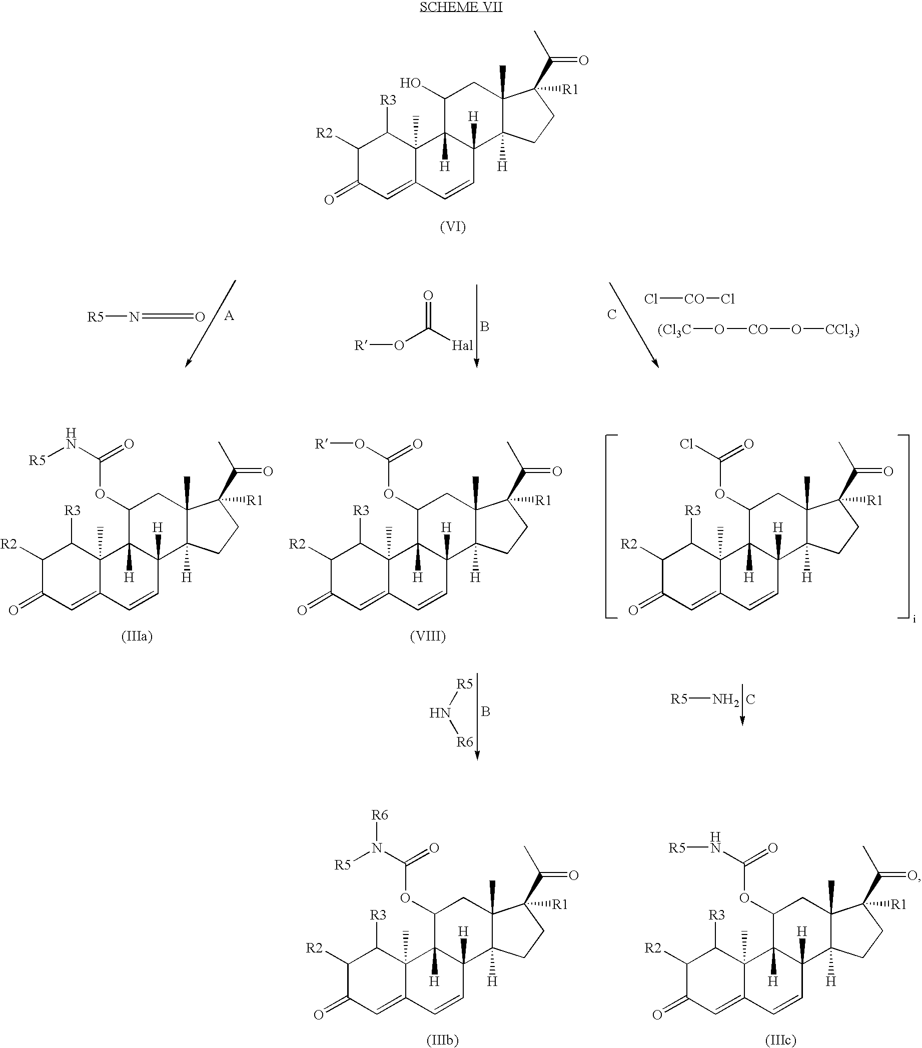

wherein R′—O— represents a suitable leaving group, and all other residues R1, R2, R3, R5 and R6 have the meanings as defined herewithin.

### Compounds of General Formula (IIIa)—Synthesis Pathway A

In order to obtain compounds of general formula (IIIa) falling under the scope of the compounds of the invention, the free C11 hydroxyl group of compounds of general formula (VI) may be reacted with an appropriately substituted isocyanate R5-N═C═O in a so called N-Hydro-C-alkoxy-addition [see e.g. Cairns & McKusick, 1950^{l}].

### Compounds of General Formula (IIIb)—Synthesis Pathway B

In order to obtain compounds of general formula (IIIb) falling under the scope of the compounds of the invention, the free C11 hydroxyl group of compounds of general formula (VI) may be converted into a intermediate carbonate group —O—CO—O—R′ at the C11 position (compounds of formula (VIII)), which group is then reacted with the appropriate primary or secondary amine R5R6-NH to deliver the desired compound with the carbamate group [ee e.g. Anderson & McGregor (1957)^{l}].

### Compounds of General Formula (IIIc)—Synthesis Pathway C

In order to obtain compounds of general formula (IIIc) falling under the scope of the compounds of the invention, the free C11 hydroxyl group of compounds of general formula (VI) may be converted by reaction with phosgene or triphosgen into an intermediate reactive chloroformate group —O—CO—Cl at the C11 position, which chloroformate group is then reacted with the appropriate primary amine R5-NH₂ to deliver the desired compound with the carbamate group in C11 [see e.g. Boden et al. (1993)^{lii}].

### C-III. Synthesis of Compounds of Formula (I) Wherein A Represents —CO-NR4-NR6-

The compounds of general formula (VI) can be converted into the corresponding 9β,10α steroidal 11α or 11β-substituted hydrazinecarboxylic acid esters of general formula (VII) as depicted in the following general SCHEME VIII.

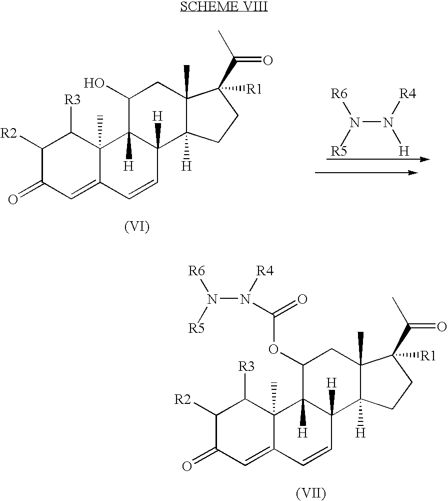

Starting compound (VI) may be converted into a reactive intermediate—a carbonate or chloroformate—as displayed in general SCHEME VII and then reacted with the appropriately substituted hydrazine R5R6-NHR4, preferably R5R6-NH₂, to deliver the desired compound of general formula (VII) [see e.g. Rosling et al, 1997^{liii}].

### C-IV. Synthesis of Compounds of Formula (I) Wherein A Represents —CO—NH—SO₂—NR6-

The compounds of general formula (VI) can be converted into the corresponding 9β,10α steroidal 11α or 11β-substituted hydrazinecarboxylic acid esters of general formula (VII) as depicted in the following general SCHEME VIII.

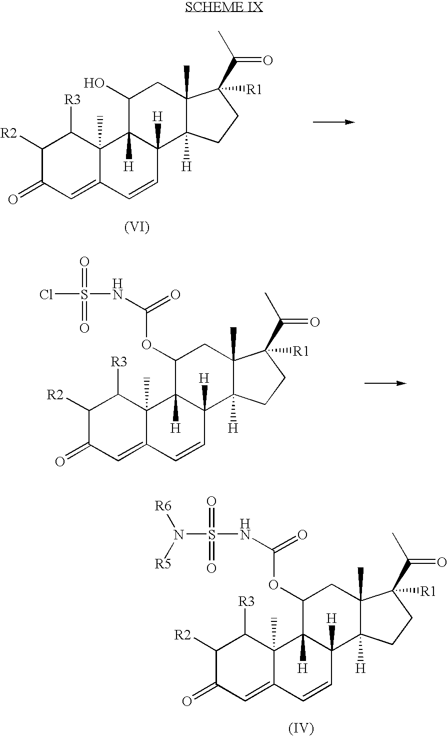

In a first step, the C11-hydroxyl group of compound (VI) may be reacted with chlorosulfonyl isocyanate to deliver the intermediary retrosteroid carrying a —O—CO—NH—SO₂—Cl group in C11 position, which is then secondly converted into the desired compound of general formula (IV) by reaction with the appropriate primary or secondary amine R5R6-NH [see e.g. Graf (1963)^{liv}].

D. Modification of the R5 Substituent Introduced in the C11 Position—Further Compounds of the Invention (I)

In case that R5 represents a substituted aryl, aryl-(C₁-C₄)alkyl, heteroaryl or heteroaryl-(C₁-C₄)alkyl group and R6 represents hydrogen, further modifications of any substituents located at the aryl or heteroaryl moiety might be carried out if necessary or desired.

### D-I. Derivatisation of a —OH Substituent in the Aryl or Heteroaryl Moiety of R5

In case that R5 comprises an aryl or heteroaryl group substituted with at least one —OH substituent, the free hydroxyl substituent may be reacted with

a) an appropriately substituted isocyanate R¹⁰—N═C═O to produce the corresponding compound with the desired R5 side chain comprising an aryl or heteroaryl group carrying at least one substituent —O—CO—NHR¹⁰, or
b) an appropriately substituted carboxylic acid R¹¹—CO—OH or a more reactive derivative thereof (e.g. an acid anhydride or an acid chloride) in an esterification reaction, to produce the corresponding compound with the desired R5 side chain comprising an aryl or heteroaryl group carrying at least one substituent —O—CO—R¹¹.

### D-II. Derivatisation of a —CH₂—OH Substituent in the Aryl or Heteroaryl Moiety of R5

In case that R5 comprises an aryl or heteroaryl group substituted with at least one —CH₂—OH group, the derivatisation may include the oxidation of the —CH₂—OH group into a carbonyl —CHO group, e.g. using a Jones reagent. Alternatively, the oxidation reaction may for example be performed using DMSO as oxidizing agent in the presence of an electrophile, for example Dicyclohexylcarbodiimide (DCC) or oxalyl chloride (so-called “Swern oxidation”). Furthermore, selective oxidation can also be performed with PCC as oxidizing agent. Another option is to perform the oxidation reaction in the presence of a catalytic amount of a stable organic nitroxyl radical. The above reaction may be carried out by electro-oxidation in the presence of the organic nitroxyl radical. Alternatively, the oxidation reaction may be carried out in the presence of a nitroxyl radical and at least one molar equivalent of a co-oxidant selected from the group consisting of m-chloroperbenzoic acid, high-valent metal salts, sodium bromite, sodium or calcium hypochlorite, N-chlorosuccinimide or hypervalent iodine compounds such as [bis(acetoxy)iodo]benzene. Preferably, the co-oxidant is sodium hypochlorite. The stable organic radical preferably comprises a completely α-substituted piperidin-1-oxy radical, such as 2,2,6,6-tetramethyl-1-piperidinyloxy, free radical (TEMPO, free radical). The resulting carbonyl function may be further functionalized (see D-V).

Another possible modification of the —CH₂—OH substituent is the reaction of the free hydroxyl group according to the procedures described within paragraph D-I.

D-III. Derivatisation of a —(C₀-C₄)alkyl-COOH substituent in the aryl or heteroaryl moiety of R5

If R5 comprises an aryl or heteroaryl group substituted with at least one —COOH or —(C₁-C₄)alkyl-COOH group, a reaction may be carried out comprising the modification of the —COOH substituent into an ester or amide derivative by nucleophilic substitution with the appropriate alcohol R⁹—OH or the appropriate amine R⁷R⁸NH by reactions well known to the skilled artisan (e.g. EDCl coupling), thereby resulting in the desired derivative of compounds of general formula (I) with a residue R5 comprising an aryl or heteroaryl group carrying at least one substituent —CO—O—R⁹ and —CO—NR⁷R⁸, respectively.

### D-IV. Derivatisation of a —NHR¹³ or —NH₂ Substituent in the Aryl or Heteroaryl Moiety of R5

In case that R5 comprises an aryl or heteroaryl group substituted with at least one —NR7R8 group being a —NHR¹³ and/or —NH₂ group, a subsequent reaction may give rise to compounds with at least one —NH—CO—R¹¹, —NH—CO—NHR¹⁰, or —NH—CO—O—R⁹, and —NR¹³—CO—R¹¹, —NR¹³—CO—NHR¹⁰, or —NR¹³—CO—O—R⁹ substituent in the aryl or heteroaryl group of R5 by reaction of the amine function —NH₂ or —NHR¹³ with an appropriately substituted acid halide R¹¹—CO-Hal, an appropriately substituted isocyanate R¹⁰—N═C═O, and an appropriately substituted chloroformic acid ester R⁹—O—CO—Cl, respectively.

### D-V. Derivatisation of a —CHO Substituent in the Aryl or Heteroaryl Moiety of R5

In case that the compounds of the invention carry a residue R5 comprising an aryl or heteroaryl group substituted with at least one —CH₂—OH group, the oxidation of said —CH₂—OH group to a carbonyl —CHO group as explained above produces a valuable starting compound for further functionalization. The derivatisation of the carbonyl function on the aryl or heteroaryl group A may give rise to a substituent selected from the group consisting of —CH═N—O—R¹², —CH═N—O—CO—NHR¹⁰, —CH═N—O—CO—R¹¹ and —CH═N—O—CO—O—R⁹, and may be performed by reactions of the carbonyl function according to the procedures described within U.S. Pat. No. 5,693,628.

For example, the carbonyl group may be reacted with a compound of general formula NH₂—O—R¹², wherein R¹² is a hydrogen atom, an —(C₁-C₄)alkyl residue, a halogenated —(C₁-C₄)alkyl residue, an aryl or an aryl-(C₁-C₄)alkyl group, producing a compound with a —CH═N—O—R¹² substituent in the aryl or heteroaryl moiety of R5, respectively. The compound of general formula NH₂—O—R¹² is present in the form of such compound, or in a form from which the compound of the general formula NH₂—O—R¹² is released under the selected conditions of the reaction. Preferably, the reaction is carried out with equimolar ratios of the corresponding educts.

A resulting compound with a —CH═N—OH substituent in the aryl or heteroaryl group of R5 may be modified further by well known reactions of the hydroxyl-imino-methyl group:
a) formation of the corresponding urethane derivative —CH═N—O—CO—NHR¹⁰ by reaction with an appropriately substituted isocyanate R¹⁰—N═C═O in inert solvents;
b) esterification to produce the corresponding —CH═N—O—CO—R¹¹ side chain by using acylating agents such as appropriately substituted acid halogenides R¹¹—CO-Hal or acid anhydrides (R¹¹—CO)₂O in the presence of bases; or
c) formation of the corresponding —CH═N—O—CO—O—R⁹ derivative by reaction with an appropriately substituted chloro-formic acid ester derivative R⁹—O—CO—Cl.

### EXPERIMENTAL SECTION

### General Experimental Conditions

In single compound synthesis all reactions were stirred magnetically or shaked with an orbital shaker unless otherwise indicated. Sensitive liquids and solutions were transferred via syringe or cannula, and introduced into reaction vessels through rubber septa, in these cases the reaction were carried out under a positive pressure of dry argon or dry nitrogen. Commercial grade reagents and solvents were used without further purification.

Unless otherwise stated, the term “concentration under reduced pressure” refers to use of a Buchi or Heidolph rotary evaporator (“Rotavapor”) or vacuum centrifuges (“GeneVac”) at approximately 15 mm of Hg. All temperatures are reported uncorrected in degrees Celsius (° C.). Unless otherwise indicated, all parts and percentages are by volume.

Thin-layer chromatography (TLC) was performed on Merck® pre-coated glass-backed silica gel or aluminium sheets 60A F-254 250 μm plates unless stated otherwise. Visualization of plates was effected by one or more of the following techniques: (a) ultraviolet illumination (254 nm or 266 nm), (b) exposure to iodine vapour or iodine vapour and phosphomolybdic acid and subsequent heating, (c) spraying of the plate with Schlittler's reagent solution followed by heating, (d) spraying of the plate with anisaldehyde solution followed by heating, and/or (e) spraying of the plate with Rauxz reagent solution followed by heating.

Preparative HPLC was performed on a binary HPLC system from Waters (pump 2525, fraction collector 2767, detector ZQ2000 singlequadruploe MS-detector).

Melting points (mp) were determined using a Reichert Thermovar melting point apparatus or a Mettler DSC822 automated melting point apparatus and are uncorrected.

Proton (1H) nuclear magnetic resonance (NMR) spectra were measured with a Bruker ARX (400 MHz) or Bruker ADVANCE (500 MHz) spectrometer with either Me₄Si (δ 0.00) or residual protonated solvent (CHCl₃ δ 7.26; CHD₂OD δ 3.30; DMSO-d₅ δ 2.50) as standard. Carbon (¹³C) NMR spectra were measured with a Bruker ARX (100 MHz) spectrometer with either Me₄Si (δ 0.00) or solvent (CDCl₃ δ 77.05; CD₃OD δ 49.0; DMSO-d₆ δ 39.45) as standard.

NMR spectra and elemental analyses of the compounds were consistent with the assigned structures.

### Important Intermediates or Reference Examples—Detailed Synthesis

### 1,2-Methylene-9β,10α-pregna-4,6-diene-3,20-dione

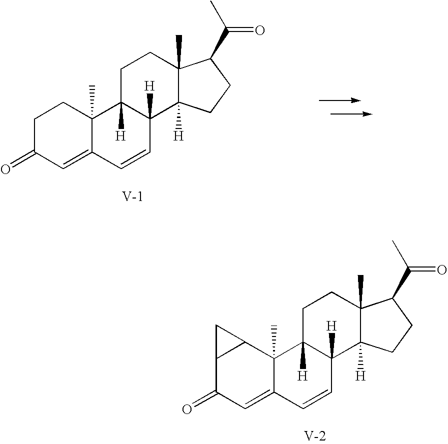

Commercially available Dydrogesterone (V-1) is converted into the corresponding 1,2-Methylene-9β,10α-pregna-4,6-diene-3,20-dione (1,2-Methylene-dydrogesterone) (V-2) by dehydrogenation and subsequent reaction with Dimethylsulfoxonium methylide or a similar donor reagent for a methylene group, as described within U.S. Pat. No. 3,937,700^{xxvii}.

The dehydrogenation reaction was carried out by stirring together 6 g Dydrogesterone (V-1), 5.8 g DDQ and 120 ml dioxane/HCl (1 mg/ml HCl) for 90 min. Then, 0.8 g CaCO₃ were carefully added and the reaction mixture stirred for another 20 min. The participate was removed by filtration and the filtrate thereafter refluxed for 90 min. After adding 1 l EtOAc, the organic layer was washed with 5% thiosulfate, 5% NaHCO₃, 5% NaOH solution and brine. After drying with Na₂SO₄ and evaporation 6.2 g crude material were obtained which were used in the next step without further purification.

To introduce the methylene group, 1.7 g NaH were dissolved in 100 ml DMSO and heated for 60 min at 60° C. Then, 13.7 g trimethyl sulfoxonium iodide were added. After 20 minutes a solution of 6.3 g Δ1,2-Dydrogensterone and 70 ml THF were added at RT and the reaction mixture was stirred for further 4 h at ambient temperature. The reaction was quenched by adding water and the reaction mixture was evaporated. The residue was dissolved in EtOAc and washed several times with brine, dried over Na₂SO₄ and evaporated to dryness under reduced pressure. Further purification by flash chromatography with EtOAc yielded 3 g of the desired product. LC-MS (ES+): rt 5.85 min, m/z (rel. Intens) 325 [(M+H)⁺, 100%]

### 9β,10α-Pregna-4-ene-3,20-dione (9β,10α-Progesterone)

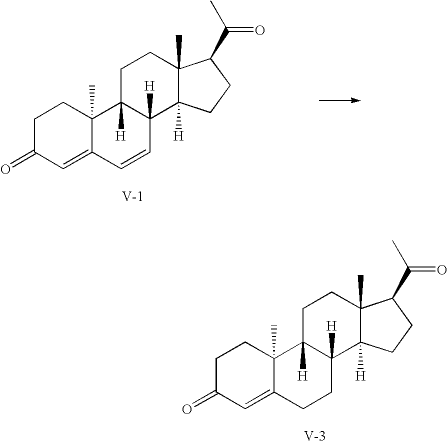

Commercially available Dydrogesterone (V-1) is converted to the corresponding 9β,10α-Progesterone (V-3) under reducing conditions.

A suspension of 0.75 g of Pd/CaCO₃ (5% Pd) in 100 ml of toluene was hydrogenated with H₂. Then a solution of 50 g of Dydrogesterone (160 mmol) in 550 ml of toluene was added; residues of Dydrogesterone were added by rinsing with 2×50 ml portions of toluene. The hydrogenation was carried out under vigorous stirring until 3.6 l of H₂ have been absorbed (approx. 1 h). The suspension was suction filtered through diatomaceous earth and rewashed with some toluene. The solvent was removed under a vacuum, and the resulting residue redissolved in approx. 90 ml of dichloromethane (DCM). The product was crystallized by addition of 900 ml of warm hexane. The crystals formed were removed by suction filtration and rewashed with 100 ml of 10% DCM/hexane. Vacuum-drying gave rise to 36.9 g of (I-3) ([α]D₂₀=−60 (c=1, CHCl₃)). The solvent was completely removed from the mother liquor and the residue (approx. 13 g) was dissolved in approximately 20 ml of DCM. Crystallization was initiated by addition of 150 ml of hexane. After suction filtration and washing, 7.3 g of secondary crystals of (1-3) were obtained. Overall yield: 44.2 g of (1-3) (88%).

### 17α-Ethoxy-9β,10α-pregna-4,6-diene-3,20-dione

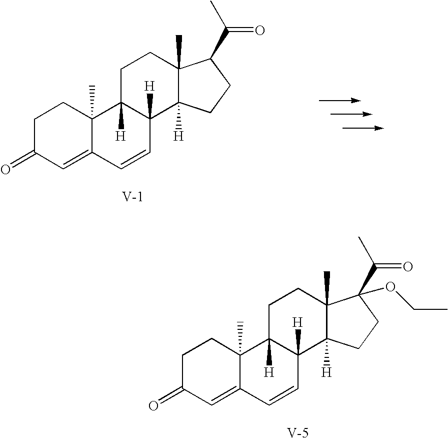

Dydrogesterone (V-1) is converted into the corresponding 17α-Ethoxy-9β,10α-pregna-4,6-diene-3,20-dione of formula (V-5) by a multi-step reaction as described in the general section, part A-III, and as displayed in SCHEME III.

### Detailed Synthesis:

To a solution of 4.1 g Dydrogesterone (V-1) dissolved in MeOH were added 3.5 g Bu₄NBH₄ in several portions. The reaction mixture was stirred over night at ambient temperature. After careful quenching of the reaction mixture with diluted HCl solution, the reaction mixture was evaporated and than diluted with EtOAc. The organic phase was washed with 1 M NaHCO₃, dried over Na₂SO₄ and evaporated to dryness under reduced pressure. The solid residue was crystallized from acetone and the mother liquor further purified by flash chromatography yielding in total to 4.8 g C20-OH-Dydrogesterone (LC-MS (ES+): rt 5.87 min, m/z (rel. Intens) 315 [(M+H)⁺, 100%]).

To a solution of 4.8 g C20-OH-Dydrogesterone and 23 ml pyridine were added dropwise 4.1 g pTosCl at a temperature between −10° C. and 0° C. The reaction mixture was kept in a fridge for 4 d. The reaction was quenched with MeOH. Thereafter the main part of methanol and pyridine was removed by evaporation under reduced pressure. The residue was dissolved in DCM and 8% H₂SO₄ solution (pH has to be below 5). After phase separation the organic layer was dried over Na₂SO₄ and evaporated to dryness under reduced pressure at a temperature below 35° C. bath temperature yielding 6.05 g 20-TosO-Dydrogesterone (contains still some solvent residues; LC-MS (ES+): rt 7.32 min). The obtained material was dissolved in 10 ml 2,4,6-collidine and keep for 5 h at 140° C. After removal of 2,4,6-collidine under reduced pressure, toluene and 3 M H₂SO₄ solution were added. The water layer was extracted twice with further toluene. The combined organic layers were dried over Na₂SO₄ and evaporated under reduced pressure to dryness yielding 3.5 g Δ17,20-dydrogesterone (LC-MS (ES+): rt 7.73 min, m/z (rel. Intens) 297 [(M+H)⁺, 100%]).

3.5 g Δ17,20-dydrogesterone was oxidized by adding 150 ml tert. butanol, 5 ml pyridine, 2.4 g NMMO, 2.5 ml 30% H₂O₂ and 100 mg OsO₄ and stirring the reaction mixture for 4 h at ambient temperature. The reaction was than quenched with Na₂S₂O₃ solution, further diluted with 1 M KHSO₄ solution and extracted several times with EtOAc. After drying the organic layer over Na₂SO₄, evaporating to dryness and further purification by flash chromatography with CH/EtOAc, 1.7 g 17α-OH-Dydrogesterone were obtained (LC-MS: retention time 5.3 minutes).

(LC-MS (ES+): rt 5.3 min, m/z (rel. Intens) 329 [(M+H)⁺, 100%]).

Under a dry N₂-atmosphere, to a suspension of 164 mg 17α-OH-Dydrogesterone and 600 μl dimethylgycol were added 310 μl diethylsulfate and 164 mg KOH (powder) at −10° C. The reaction mixture was stirred over night at ambient temperature. The reaction mixture was diluted with DCM and water. The organic layer was separated, dried over Na₂SO₄ and evaporated under reduced pressure to dryness yielding 385 mg of the crude product, which was further purified by flash chromatography (eluent CH/EtOAc 100/1→100/5). 111 mg pure 17α-Ethoxy-dydrogesterone were obtained (LC-MS (ES+): rt 6.83 min, m/z (rel. Intens) 357 [(M+H)⁺, 100%]).

### 17α-Ethoxy-1,2-methylene-9β,10α-pregna-4,6-diene-3,20-dione

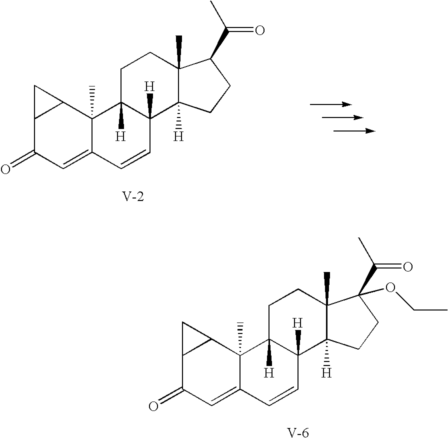

1,2-Methylene-dydrogesterone (V-2) is converted into the corresponding 17α-Ethoxy-1,2-methylene-9β,10α-pregna-4-ene-3,20-dione (V-6) according to the protocols displayed hereinabove.

### 11β-Hydroxy-dydrogesterone Derivatives of General Formula (VI)

The following 11β hydroxylated Dydrogesterone derivatives were obtained starting from the educts disclosed above by microbial hydroxylation as fully described within international patent application WO2007082891^{xlix}.

### 11β-Hydroxy-dydrogesterone (VI-1)

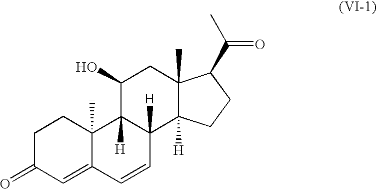

¹H NMR (400 MHz, CDCl₃): δ ppm 0.9 (s, 3H) 1.2 (s, 3H) 1.4-1.5 (m, 1H) 1.7-2.0 (m, 6H) 2.2 (s, 3H) 2.2-2.3 (m, 1H) 2.3-2.4 (m, 2H) 2.4-2.6 (m, 3H) 2.7 (ddd, J=12.0, 5.8, 5.6 Hz, 1H) 4.4-4.5 (m, 1H) 5.7-5.7 (m, 1H) 6.2-6.2 (m, 2H)

¹³C NMR (101 MHz, CDCl₃): δ ppm 14.7 (q, 1C) 21.7 (q, 1C) 22.6 (t, 1C) 24.8 (t, 1C) 31.2 (q, 1C) 33.7 (t, 1C) 35.2 (d, 2C) 35.7 (s, 1C) 43.4 (s, 1C) 46.2 (t, 1C) 49.7 (d, 1C) 49.9 (d, 1C) 63.6 (d, 1C) 67.6 (d, 1C) 124.2 (d, 1C) 126.9 (d, 1C) 140.2 (d, 1C) 162.2 (s, 1C) 199.1 (s, 1C) 208.6 (s, 1C)

### 17α-Ethoxy-11β-hydroxy-9β,10α-pregna-4,6-diene-3,20-dione (VI-5)

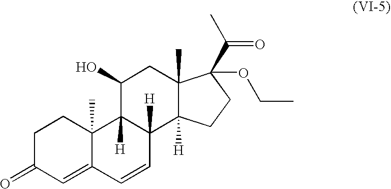

¹H NMR (400 MHz, CDCl₃): δ ppm 0.86 (s, 3H) 1.15 (t, J=6.9 Hz, 3H) 1.28 (s, 3H) 1.37-1.52 (m, 1H) 1.64-1.94 (m, 5H) 2.15 (s, 3H) 2.27-2.68 (m, 6H) 2.70-2.79 (m, 1H) 2.97-3.07 (m, 1H) 3.39-3.52 (m, 1H) 4.48-4.54 (m, 1H) 5.70-5.73 (m, 1H) 6.18-6.26 (m, 2H)

¹³C NMR (101 MHz, CDCl₃): δ ppm 15.6 (q, 1C) 16.3 (q, 1C) 21.5 (q, 1C) 23.3 (t, 1C) 24.2 (t, 1C) 26.4 (q, 1C) 33.8 (t, 1C) 35.1 (d, 1C) 35.4 (t, 1C) 35.7 (s, 1C) 38.0 (d, 1C) 44.7 (t, 1C) 47.0 (s, 1C) 50.1 (d, 1C) 59.8 (t, 1C) 68.2 (d, 1C) 95.7 (s, 1C) 124.1 (d, 1C) 127.0 (d, 1C) 140.6 (d, 1C) 162.5 (s, 1C) 199.1 (s, 1C) 210.3 (s, 1C)

### 17α-Ethoxy-11β-hydroxy-1,2-methylen-dydrogesterone (VI-6)

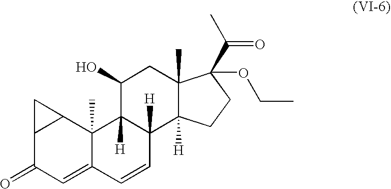

¹H NMR (501 MHz, CDCl₃): δ ppm 0.87 (s, 3H) 0.89-0.93 (m, 1H) 1.13-1.18 (m, 3H) 1.30-1.36 (m, 1H) 1.39 (s, 3H) 1.40-1.49 (m, 1H) 1.69-1.83 (m, 3H) 1.87-1.94 (m, 1H) 1.95-2.01 (m, 1H) 2.11-2.17 (m, 4H) 2.39-2.47 (m, 1H) 2.49-2.55 (m, 1H) 2.59 (dd, J=14.5, 4.4 Hz, 1H) 2.70-2.77 (m, 1H) 2.98-3.06 (m, 1H) 3.39-3.47 (m, 1 H) 4.70-4.75 (m, J=2.4 Hz, 1H) 5.51-5.53 (m, 1H) 6.08-6.10 (m, 2H)

¹³C NMR (126 MHz, CDCl₃): δ ppm 13.8 (q, 1C) 15.6 (q, 1C) 16.4 (q, 1C) 23.3 (t, 1C) 24.2 (t, 1C) 25.4 (d, 1C) 26.5 (q, 1C) 26.7 (d, 1C) 28.6 (q, 1C) 34.9 (d, 1C) 36.9 (s, 1C) 37.9 (t, 1C) 44.2 (d, 1C) 47.6 (s, 1C) 48.1 (d, 1C) 59.9 (t, 1C) 69.3 (d, 1C) 95.8 (s, 1C) 120.3 (d, 1C) 127.2 (d, 1C) 139.0 (d, 1C) 156.2 (s, 1C) 198.3 (s, 1C) 210.3 (s, 1C)

### Dydrogesterone-11β-yl 4-nitrophenyl carbonate (VII-1)

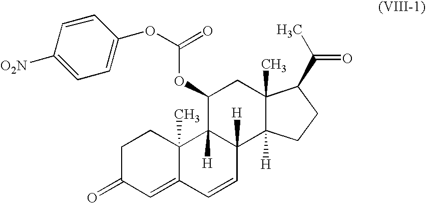

To a solution of 11β-hydroxydydrogesterone (VI-1) (328 mg, 1.00 mmol) in DCM (15 ml) and pyridine (0.6 ml) was slowly added a solution of 4-nitrophenylchloroformate (400 mg, 1.98 mmol) in DCM at 0° C. The reaction mixture was stirred at RT over night. After dilution with DCM the organic phase was washed successively with 1M aq KHSO₄ solution and 5% aq NaHCO₃ solution, dried over Na₂SO₄, filtered and concentrated in vacuo to yield the carbonate (577 mg, quant). The product of formula (VIII-1) was used for subsequent reactions without further purification. LC-MS (ES+): rt 6.26 min, m/z (rel. Intens) 494 [(M+H)⁺, 100%]

### 17-Ethoxy-dydrogesterone-11β-yl 4-nitrophenyl carbonate (VII-2)

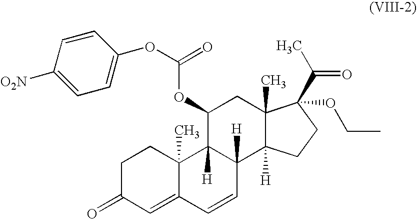

Intermediate compound (VIII-2) was prepared according to the procedure described for Dydrogesterone-11β-yl 4-nitrophenyl carbonate (VIII-1) starting from 17-Ethoxy-11β-hydroxy dydrogesterone (VI-5). LC-MS (Es+): rt 6.86 min, m/z (rel. Intens) 539 [(M+H)+, 100%]

### EXAMPLES

### Detailed Synthesis

In order to more fully illustrate the nature of the invention and the manner of practicing the same, the following examples are presented, but they should not be taken as limiting.

In principle, the reaction conditions for the examples presented below are chosen from one of the following General Procedures (according to SCHEME VI or SCHEME VII) unless explicitly indicated otherwise.

### General Procedure A (According to SCHEME VII)

To a solution of the respective 11β-Hydroxy-dydrogesterone (VI) in dry DCM/THF was added copper (I) bromide (1 eq.) under stirring. Isocyanate R5-N═C═O (3.3-4.4 eq) was added dropwise to the resulting suspension. The mixture was stirred at RT. After consumption of the starting material the reaction mixture was diluted with DCM and the organic phase was washed with water. The organic layer was dried over Na₂SO₄, filtered and concentrated in vacuo. The crude product was purified by column chromatography (silica gel).

### General Procedure B (According to SCHEME VII)

The crude nitrophenyl carbonate of the respective 11β-Hydroxy-dydrogesterone (VIII) was dissolved in DCM and an excess of the respective amine R5R6-NH, pyridine (where appropriate) and DMAP (catalytic amount) was added. The reaction mixture was stirred at RT or heated to reflux. The progress of the reaction was followed by HPLC. After dilution with DCM the organic phase was washed successively with water and 1M KHSO₄ solution, dried over Na₂SO₄, filtered and concentrated in vacuo. The crude product was purified by column chromatography (silica gel).

### General Procedure C-1 (According to SCHEME VII)

To a solution of the respective 11β-Hydroxy-dydrogesterone (VI) in DCM/toluene was added pyridine and triphosgene. The mixture was stirred at RT for 30-45 min. Then the respective amine R5-NH₂ and pyridine were added and the reaction mixture was stirred at RT. After dilution with DCM the organic phase was washed successively with water, 1M aq KHSO₄ solution and brine, dried over Na₂SO₄, filtered and concentrated in vacuo. The crude product was purified by column chromatography (silica gel).

### General Procedure C-2 (According to SCHEME VII)

To a solution of the respective 11β-Hydroxy-dydrogesterone (VI) in dry DCM was added DMAP and triphosgene. The mixture was stirred at RT for 50 min. Then the respective amine R5-NH₂ and DMAP were added and the reaction mixture was stirred at RT. After dilution with DCM the organic phase was washed successively with 1M aq KHSO₄ solution, 1M aq NaHCO₃ solution and brine, dried over Na₂SO₄, filtered and concentrated in vacuo. The crude product was purified by column chromatography (silica gel).

### General Procedure D for the Synthesis of Compounds of Formula (II) (According to SCHEME VI)

The respective 11β-Hydroxy-dydrogesterone (VI) was dissolved in dry pyridine. Then, the respective acyl chloride R5-CO—Cl (1.8-4 eq) was added at 0° C. The mixture was stirred at RT until TLC showed completion of the reaction. The reaction mixture was diluted with DCM and extracted with 1 M aq KHSO₄ solution. The organic layer war dried over Na₂SO₄, filtered and concentrated in vacuo. The crude product was purified by column chromatography (silica gel).

### Dydrogesterone-11β-yl isopropylcarbamate (Compound No. 1)

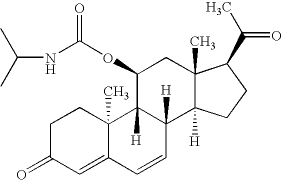

According to the General Procedure A, 11β-hydroxydydrogesterone (VI-1) (150 mg, 0.457 mmol) was treated with isoproylisocyanate (178 μL, 1.53 mmol) and copper (I) bromide (66 mg, 0.46 mmol). The mixture was stirred at RT for 3 d. After work-up the crude product was purified by column chromatography (eluent CH/EtOAc 1:2) to yield the carbamate (86 mg, 46%).

LC-MS (ES+): rt 5.53 min, m/z (rel. Intens) 414 [(M+H)⁺, 100%]

¹³C NMR (100.6 MHz, CDCl₃) δ (ppm) 14.4, 22.2, 22.6, 22.8, 23.6, 24.9, 31.5, 33.9, 35.3, 35.7, 36.0, 42.1, 42.8, 43.3, 43.7, 47.3, 49.8, 63.5, 70.1, 124.5, 127.2, 139.4, 154.6, 161.7, 199.1, 208.4.

### Dydrogesterone-11β-yl benzyl-carbamate (Compound No. 2)

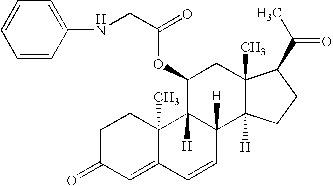

According to the General Procedure A, 11β-hydroxydydrogesterone (VI-1) (100 mg, 0.305 mmol) was treated with benzylisocyanate (164 μL, 1.33 mmol) and copper (I) bromide (44 mg, 0.306 mmol). The mixture was stirred at RT for 2 d. After work-up the crude product was purified by column chromatography (eluent CH/EtOAc 2:1 to 1:1) to yield the carbamate (30 mg, 21%).

LC-MS (ES+): rt 5.82 min, m/z (rel. Intens) 462 [(M+H)⁺, 100%]

¹H NMR (400 MHz, CDCl₃) δ (ppm) 0.80 (s, 3H), 1.27 (s, 3H), 1.35-1.43 (m, 1H), 1.68-2.00 (m, 6H), 2.08-2.28 (m, 4H), 2.39-2.61 (m, 6H), 4.31-4.41 (m, 2H), 4.91-4.94 (m, 1H), 5.48 (broad s, 1H), 5.70 (s, 1H), 6.18 (broad s, 2H), 7.25-7.34 (m, 5H).

¹³C NMR (100.6 MHz, CDCl₃) δ (ppm) 14.2, 22.0, 22.4, 24.7, 31.3, 33.7, 35.2, 35.5, 35.8, 43.0, 43.5, 45.1, 47.1, 49.6, 63.3, 70.6, 124.4, 127.0, 127.46, 127.52, 128.6, 138.2, 139.1, 155.3, 161.4, 198.9, 208.1.

### Dydrogesterone-11β-yl diethylcarbamate (Compound No. 3)

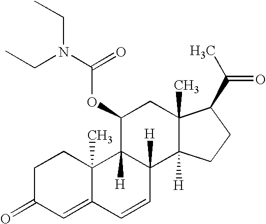

According to the General Procedure B, nitrophenyl carbonate (VIII-1) (97 mg, 0.197 mmol) dissolved in DCM (2 ml) was treated with diethylamine (100 μl, 0.957 mmol) and DMAP (cat). The mixture was stirred at RT for 4 d. After work-up the crude product was purified by column chromatography (eluent CH/EtOAc 1:1) to yield the carbamate (25 mg, 30%).

LC-MS (ES+): rt 6.01 min, m/z (rel. Intens) 427 [(M+H)⁺, 55%], 311 [(M-Et₂NCO₂⁻)⁺, 100%]

¹H NMR (400 MHz, CDCl₃) δ (ppm) 0.86 (s, 3H), 1.02-1.17 (m, 6H), 1.28 (s, 3H), 1.39-1.48 (m, 1H), 1.70-2.03 (m, 6H), 2.15 (s, 3H), 2.19-2.68 (m, 7H), 3.15-3.40 (m, 4H), 5.50 (m_{c}, 1H), 5.70 (s, 1H), 6.17-6.22 (m, 2H).

¹³C NMR (100.6 MHz, CDCl₃) δ (ppm) 13.4, 14.1, 22.1, 22.5, 24.7, 26.8, 31.3, 33.7, 35.2, 35.5, 35.9, 40.9, 41.7, 43.0, 43.8, 47.5, 49.6, 63.3, 70.3, 124.4, 127.1, 139.2, 154.8, 161.5, 199.0, 208.3.

### Morpholine-4-carboxylic acid dydrogesterone-11β-yl ester (Compound No. 4)

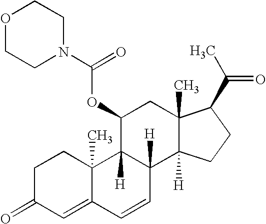

According to the General Procedure B, nitrophenyl carbonate (VIII-1) (82 mg, 0.167 mmol) dissolved in DCM was treated with morpholine (0.5 ml). The mixture was heated to reflux for 2 h and then stirred at RT over night. After work-up the crude product was purified by column chromatography (eluent DCM/methanol 97:3) to yield the carbamate (25 mg, 34%).

LC-MS (ES+): rt 5.21 min, m/z (rel. Intens) 442 [(M+H)⁺, 100%]

¹³C NMR (100.6 MHz, CDCl₃) δ (ppm) 14.5, 22.0, 22.6, 24.7, 31.4, 33.7, 35.1, 35.5, 35.9, 43.0, 43.5, 44.0, 47.2, 49.5, 63.2, 66.4, 71.1, 124.5, 127.1, 139.0, 154.3, 161.3, 198.9, 208.2.

### (2-Hydroxyethyl)-methyl-carbamic acid dydrogesterone-11β-yl ester (Compound No. 5)

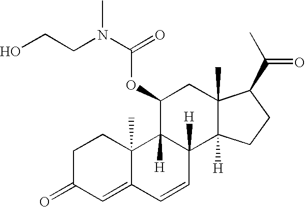

According to the General Procedure B, nitrophenyl carbonate (VIII-1) (67 mg, 0.136 mmol) dissolved in DCM was treated with methylaminoethanol (0.3 ml) and DMAP (cat). The mixture was stirred at RT for 1d. After work-up the crude product was purified by column chromatography (eluent DCM/MeOH 97:3) to yield the carbamate (22 mg, 38 LC-MS (ES+): rt 4.76 min, m/z (rel. Intens) 430 [(M+H)⁺, 40%], 311 [(M−HOCH₂CH₂(CH₃)NCO₂)⁺, 100%]

¹H NMR (400 MHz, CDCl₃) δ (ppm) 0.87 (s, 3H), 1.27 (s, 3H), 1.37-1.47 (m, 1H), 1.71-2.00 (m, 7H), 2.15 (s, 3H), 2.20-2.68 (m, 7H), 2.87-3.02 (broad s, 3H), 3.40-3.50 (m, 2H), 3.61-3.84 (m, 2H), 5.49 (broad s, 1H), 5.70 (s, 1H), 6.16-6.22 (m, 2H).

¹³C NMR (100.6 MHz, CDCl₃) δ (ppm) 14.4, 22.1, 22.5, 24.7, 31.3, 33.7, 35.1, 35.3, 35.4, 35.9, 43.0, 43.6, 47.3, 49.5, 51.9, 61.2, 63.3, 71.1, 124.4, 127.1, 139.2, 156.7, 161.5, 199.0, 208.3.

### Butyl-methyl-carbamic acid dydrogesterone-11β-yl ester (Compound No. 6)

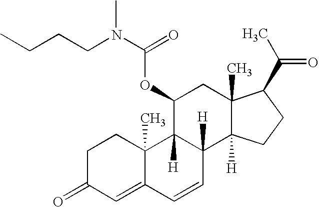

According to the General Procedure B, nitrophenyl carbonate (VIII-1) (147 mg, 0.299 mmol) dissolved in DCM was treated with N-methylbutylamine (0.14 ml), pyridine (0.3 ml) and DMAP (cat). The mixture was stirred at RT for 11 d. After work-up the crude product was purified by column chromatography (eluent DCM/MeOH 97:3) to yield the carbamate (18 mg, 14%).

LC-MS (ES+): rt 6.36 min, m/z (rel. Intens) 442 [(M+H)⁺, 50%], 311 [(M-CH₃CH₂CH₂CH₂(CH₃)NCO₂⁻)⁺, 100%].

¹H NMR (400 MHz, CDCl₃) δ (ppm) 0.86-0.92 (m, 5H), 1.24-1.33 (m, 6H), 1.39-1.47 (m, 3H), 1.69-1.99 (m, 6H), 2.14 (s, 3H), 2.21-2.70 (m, 7H), 2.82-2.89 (m, 3H), 3.08-3.35 (m, 2H), 5.49 (broad s, 1H), 5.70 (s, 1H), 6.16-6.22 (m, 2H).

¹³C NMR (100.6 MHz, CDCl₃) δ (ppm) 13.7, 14.3, 19.8, 22.1, 22.5, 24.7, 26.8, 29.6, 30.1, 31.3, 34.5, 35.2, 35.5, 35.9, 43.1, 43.7, 47.4, 48.4, 48.9, 49.6, 63.3, 70.6, 124.5, 127.1, 139.2, 155.3, 161.5, 199.0, 208.2.

### Methyl-(1-methyl-piperidin-4-yl)-carbamic acid dydrogesterone-11-yl ester (Compound No. 7)

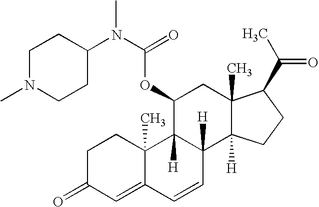

According to the General Procedure B, nitrophenyl carbonate (VIII-1) (246 mg, 0.500 mmol) dissolved in DCM was treated with 1-methyl-4-(methylamino)-piperidine (290 μl, 2.00 mmol), pyridine (0.3 ml) and DMAP (cat). The mixture was stirred at RT for 4 d. After work-up the crude product was purified by column chromatography (eluent EtOAc) to yield the carbamate (14 mg, 6%).

LC-MS (ES+): rt 4.34 min, m/z (rel. Intens) 483 [(M+H)⁺, 100%]

¹³C NMR (100.6 MHz, CDCl₃) δ (ppm) 14.4, 22.1, 22.1, 22.6, 24.7, 28.3, 28.9, 29.6, 31.4, 33.7, 35.1, 35.5, 35.9, 43.0, 43.7, 45.8, 47.3, 49.6, 55.0, 63.3, 70.6, 124.5, 127.1, 139.2, 155.3, 161.5, 199.0, 208.3.

### Phenyl-carbamic acid dydrogesterone-11β-yl ester (Compound No. 8)

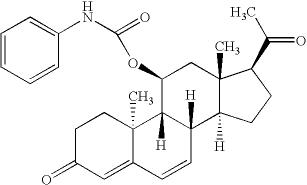

According to the General Procedure B, nitrophenyl carbonate (VIII-1) (94 mg, 0.190 mmol) dissolved in DCM was treated with aniline (0.1 ml), pyridine (0.3 ml) and DMAP (cat). The mixture was stirred at RT for 5 d. After work-up the crude product was purified by column chromatography (eluent CH/EtOAc) to yield the carbamate (25 mg, 29%).

LC-MS (ES+): rt 5.95 min, m/z (rel. Intens) 448 [(M+H)⁺, 100%]

¹H NMR (400 MHz, CDCl₃) δ (ppm) 0.85 (s, 3H), 1.29 (s, 3H), 1.37-1.48 (m, 1H), 1.71-2.06 (m, 6H), 2.14 (m, 3H), 2.21-2.30 (m, 1H), 2.44-2.69 (m, 6H), 5.55 (broad s, 1H), 5.71 (s, 1H), 6.19 (s, 2H), 6.51 (broad s, 1H), 7.05-7.10 (m, 1H), 7.27-7.37 (m, 4H).

¹³C NMR (100.6 MHz, CDCl₃) δ (ppm) 14.3, 22.0, 22.5, 24.7, 31.3, 33.7, 35.2, 35.5, 35.8, 43.0, 43.5, 47.2, 49.6, 63.3, 70.9, 118.8, 123.7, 124.5, 127.1, 129.0, 137.5, 139.0, 152.3, 161.3, 198.9, 208.1.

### (3-Hydroxy-phenyl)-carbamic acid dydrogesterone-11β-yl ester (Compound No. 9)

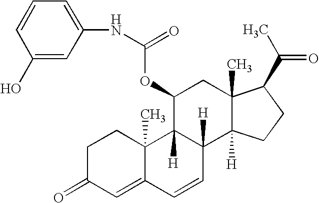

According to the General Procedure B, nitrophenyl carbonate (VIII-1) (294 mg, 0.598 mmol) dissolved in DCM was treated with 3-hydroxyaniline (262 mg, 2.40 mmol), pyridine (0.3 ml) and DMAP (cat). The mixture was stirred at RT for 12 d. After work-up the crude product was purified by column chromatography (eluent CH/EtOAc 2:1 to 1:2) to yield the carbamate (153 mg, 55%).

LC-MS (ES+): rt 5.68 min, m/z (rel. Intens) 464 [(M+H)⁺, 100%]

¹H NMR (400 MHz, CDCl₃) δ (ppm) 0.85 (s, 3H), 1.28 (s, 3H), 1.37-1.49 (m, 1H), 1.73-2.02 (m, 6H), 2.15 (s, 3H), 2.20-2.73 (m, 7H), 3.80 (broad s, 2H), 5.42 (broad s, 1H), 5.72 (s, 1H), 6.20 (s, 2H), 6.45 (t, J=2.3 Hz, 1H), 6.49-6.54 (m, 2H), 7.11 (t, J=8.1 Hz, 1H).

¹³C NMR (100.6 MHz, CDCl₃) δ (ppm) 12.3, 20.4, 20.9, 22.9, 29.5, 31.9, 33.4, 33.6, 34.0, 41.1, 41.3, 45.3, 47.7, 61.5, 73.4, 105.8, 108.8, 111.0, 122.8, 125.3, 128.3, 137.3, 146.1, 105.2, 151.0, 159.4, 197.0, 206.4.

### (2-Methoxy-phenyl)-carbamic acid dydrogesterone-11β-yl ester (Compound No. 10)

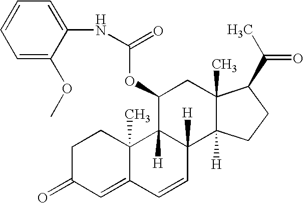

According to the General Procedure B, nitrophenyl carbonate (VIII-1) (246 mg, 0.500 mmol) dissolved in DCM was treated with o-anisidine (225 μl, 2.00 mmol), pyridine (0.3 ml) and DMAP (cat). The mixture was stirred at RT for 7 d. After work-up the crude product was purified by column chromatography (eluent CH/EtOAc) to yield the carbamate (43 mg, 18%).

LC-MS (ES+): rt 4.23 min, m/z (rel. Intens) 478 [(M+H)⁺, 100%]

¹³C NMR (100.6 MHz, CDCl₃) δ (ppm) 14.3, 22.0, 22.5, 24.8, 31.3, 33.7, 35.2, 35.5, 35.8, 43.1, 43.5, 47.1, 49.6, 55.6, 63.3, 70.7, 110.0, 118.3, 121.0, 123.0, 124.5, 127.1, 127.2, 139.1, 147.6, 152.2, 161.4, 198.9, 208.1.

### (4-Methoxy-phenyl)-carbamic acid dydrogesterone-11β-yl ester (Compound No. 11)

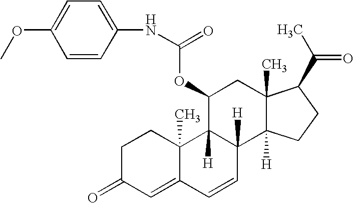

According to the General Procedure B, nitrophenyl carbonate (VIII-1) (147 mg, 0.299 mmol) dissolved in DCM was treated with p-anisidine (148 mg, 1.20 mmol), pyridine (0.3 ml) and DMAP (cat). The mixture was stirred at RT for 5 d. After work-up the crude product was purified by column chromatography (eluent CH/EtOAc 2:1 to 1:1) to yield the carbamate (74 mg, 52%).

LC-MS (ES+): rt 5.84 min, m/z (rel. Intens) 476 [(M+H)⁺, 100%]

¹³C NMR (100.6 MHz, CDCl₃) δ (ppm) 14.3, 22.0, 22.5, 24.7, 31.3, 33.7, 35.2, 35.5, 35.8, 43.0, 43.5, 47.2, 49.6, 55.4, 63.3, 70.7, 114.2, 120.8, 124.5, 127.1, 130.5, 139.1, 152.8, 156.2, 161.4, 198.9, 208.12.

### (3-Methoxy-phenyl)-carbamic acid 17-ethoxy-dydrogesterone-11β-yl ester (Compound No. 12)

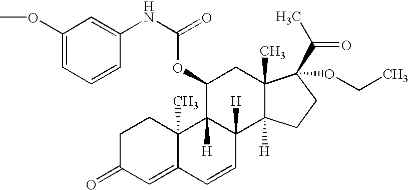

According to the General Procedure B, 17-Ethoxy-dydrogesterone-11β-yl 4-nitrophenyl carbonate (VIII-2) (575 mg, 1.07 mmol) dissolved in DCM was treated with m-anisidine (493 mg, 4.00 mmol), pyridine (1 mL) and DMAP (cat). The mixture was stirred at RT for 7 d. After work-up the crude product was purified by column chromatography (eluent CH/EtOAc 3:1) to yield the pure carbamate (102 mg, 20%) and 115 mg of a mixture of carbamate and N,N′-bis(3-methoxyphenyl)urea. Preparative HPLC (Varian Inertsil C18 50×21 mm, 3 μm; gradient 95% water+0.1 formic acid to 100% CH₃CN+0.1% formic acid) of the mixture gave the pure carbamate (57 mg, 10%).

LC-MS (ES+): rt 6.65 min, m/z (rel. Intens) 522 [(M+H)⁺, 100%]

¹H NMR (400 MHz, CDCl₃) δ (ppm) 0.79 (s, 3H), 1.16 (t, J=6.9 Hz, 3H), 1.33 (s, 3H), 1.39-1.47 (m, 1H), 1.73-2.01 (m, 5H), 2.14 (s, 3H), 2.43-2.70 (m, 7H), 2.98-3.04 (m, 1H), 3.41-3.47 (m, 1H), 3.79 (s, 3H), 5.59 (broad s, 1H), 5.71 (s, 1H), 6.17-6.22 (m, 2H), 6.49 (s, 1H), 6.60-6.63 (m, 1H), 6.85-6.87 (m, 1H), 7.08-7.09 (m, 1H), 7.19 (t, J=8.2 Hz, 1H).

¹³C NMR (100.6 MHz, CDCl₃) δ (ppm) 15.6, 16.0, 21.8, 23.3, 24.2, 26.5, 33.8, 35.3, 35.4, 35.6, 35.8, 44.4, 46.8, 47.3, 55.3, 59.9, 71.8, 95.5, 104.5, 109.4, 111.0, 124.5, 127.2, 129.8, 138.9, 139.6, 152.4, 160.3, 162.0, 199.2, 210.3.

### Benzo[1,3]dioxol-5-yl-carbamic acid dydrogesterone-11β-yl ester (Compound No. 13)

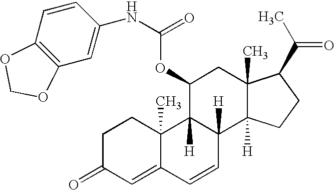

According to the General Procedure C-1, 11β-Hydroxy-dydrogesterone (VI-1) (800 mg, 2.44 mmol) dissolved in toluene/DCM (10/25 mL) was stirred with triphosgen (289 mg, 0.976 mmol) and pyridine (236 μl) for 30 min. Then 3,4-methylendioxyaniline (395 mg, 2.88 mmol) and pyridine (1.5 ml) was added and the reaction mixture was stirred at RT over night. After work-up the crude product was purified by column chromatography (eluent CH/EtOAc 2:1 to 1:1) to yield the carbamate (980 mg, 83%).

LC-MS (ES+): rt 5.96 min, m/z (rel. Intens) 492 [(M+H)⁺, 100%]

¹H NMR (400 MHz, CDCl₃) δ (ppm) 0.83 (s, 3H), 1.28 (s, 3H), 1.39-1.47 (m, 1H), 1.70-2.01 (m, 6H), 2.14 (s, 3H), 2.20-2.30 (m, 1H), 2.42-2.67 (m, 6H), 5.52 (broad s, 1H), 5.71 (s, 1H), 5.93 (s, 2H), 6.18 (s, 2H), 6.48 (broad s, 1H), 6.66-6.73 (m, 2H), 7.04 (broad s, 1H).

¹³C NMR (100.6 MHz, CDCl₃) δ (ppm) 14.4, 22.2, 22.6, 24.9, 31.4, 33.8, 35.3, 35.6, 35.9, 43.2, 43.6, 47.3, 49.7, 63.4, 70.9, 101.3, 102.0, 108.1, 112.3, 124.6, 127.2, 131.8, 139.2, 144.1, 148.0, 152.7, 161.5, 199.0, 208.2.

### (3-Carbamoyl-phenyl)-carbamic acid dydrogesterone-11β-yl ester (Compound No. 14)

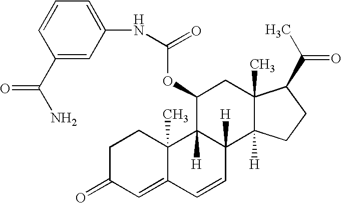

According to the General Procedure C-1, 11β-hydroxydydrogesterone (VI-1) (100 mg, 0.305 mmol) dissolved in toluene/DCM was stirred with triphosgen (89 mg, 0.305 mmol) and pyridine (72 μl, 0.915 mmol) for 20 min. After work-up the crude product was dissolved in DMF and 3-aminobenzamide (136 mg, 1.00 mmol) and pyridine (0.3 ml) was added and the reaction mixture was stirred at RT for 3 h. After work-up the crude product was purified by column chromatography (eluent EtOAc) to yield the carbamate (88 mg, 59

LC-MS (ES+): rt 4.94 min, m/z (rel. Intens) 491 [(M+H)⁺, 100%]

¹³C NMR (100.6 MHz, CDCl₃) δ (ppm) 12.6, 20.3, 20.8, 23.0, 29.5, 31.9, 33.4, 33.7, 34.1, 41.3, 41.7, 45.4, 47.8, 61.5, 69.3, 116.0, 120.3, 122.7, 125.3, 127.5, 132.4, 136.6, 137.4, 150.7, 159.7, 160.7, 167.1, 197.2, 206.5.

### (3-Methoxy-phenyl)-carbamic acid dydrogesterone-11β-yl ester (Compound No. 15)

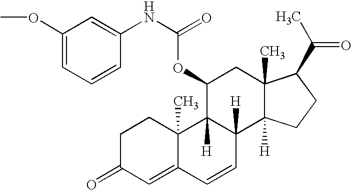

According to the General Procedure C-1, 11β-Hydroxy-dydrogesterone (VI-1) (200 mg, 0.610 mmol) dissolved in toluene/DCM was stirred with triphosgen (89 mg, 0.305 mmol) and pyridine (72 μl, 0.915 mmol) for 45 min. Then m-anisidine (0.15 mL) and pyridine (0.5 ml) was added and the reaction mixture was stirred at RT for 6 h. After work-up the crude product was purified by column chromatography (eluent CH/EtOAc 3:1 to 1:1) to yield the carbamate (186 mg, 64%).

LC-MS (ES+): rt 5.97 min, m/z (rel. Intens) 478 [(M+H)⁺, 100%]

¹H NMR (400 MHz, CDCl₃) δ (ppm) 0.84 (s, 3H), 1.36-1.47 (m, 1H), 1.69-2.01 (m, 6H), 2.14 (s, 3H), 2.20-2.29 (m, 1H), 2.42-2.67 (m, 6H), 3.79 (s, 3H), 5.53 (broad s, 1H), 5.70 (s, 1H), 6.18 (s, 2H), 6.60-6.62 (m, 1H), 6.77 (s, 1H), 6.67-6.89 (m, 1H), 7.10 (m, 1H), 7.18 (t, J=8.1 Hz, 1H).

¹³C NMR (100.6 MHz, CDCl₃) δ (ppm) 14.3, 22.0, 22.5, 24.7, 31.3, 33.6, 35.2, 35.5, 35.8, 43.0, 43.5, 47.1, 49.6, 55.2, 63.3, 70.8, 104.6, 109.3, 111.0, 124.4, 127.0, 129.7, 138.9, 139.1, 152.2, 160.2, 161.4, 198.9, 208.1.

### Ethyl-carbamic acid 3-(dydrogesterone-11β-yl-oxy-carbonylamino)-phenyl ester (Compound No 16)

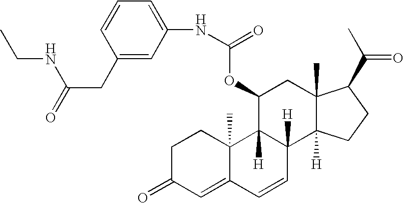

(3-Hydroxy-phenyl)-carbamic acid dydrogesterone-11β-yl ester (Compound No. 9) (66 mg, 0.142 mmol) dissolved in ACN (2 ml), ethylisocyanate (60 μl, 0.733 mmol) and triethylamine (5 μl) were mixed and heated to 65° C. The solvent was evaporated in vacuo and the crude product was purified by column chromatography (SiO₂, CH/EtOAc 1:2) to yield the carbamate (42 mg, 55%).

LC-MS (ES+): rt 5.54 min, m/z (rel. Intens) 535 [(M+H)⁺, 100%]

¹H NMR (400 MHz, CDCl₃) δ (ppm) 0.84 (s, 3H), 1.10 (t, J=7.1 Hz, 3H), 1.27 (s, 3H), 1.39-1.49 (m, 1H), 1.73-2.20 (m, 5H), 2.09-2.11 (m, 1H), 2.16 (s, 3H), 2.20-2.60 (m, 6H), 2.67-2.73 (m, 1H), 3.20-3.26 (m, 2H), 5.33 (t, J=5.3 Hz, 1H), 5.40 (s, 1H), 5.71 (s, 1H), 6.18-6.24 (m, 2H), 6.74-6.77 (m, 1H), 7.02-7.04 (m, 1H), 7.20 (t, J=8.1 Hz, 1H), 7.36-7.40 (m, 2H).

¹³C NMR (100.6 MHz, CDCl₃) δ (ppm) 12.3, 13.5, 20.4, 20.8, 22.9, 29.5, 31.9, 33.1, 33.3, 33.5, 33.5, 34.0, 41.1, 41.3, 47.7, 61.5, 73.5, 110.4, 113.0, 114.8, 122.7, 125.3, 127.8, 137.6, 138.9, 149.7, 151.0, 153.8, 159.9, 197.4, 206.7.

### Acetic acid dydrogesterone-11β-yl ester (Reference Compound)

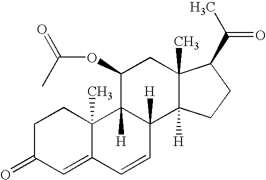

According to the General Procedure D, 11-hydroxydydrogesterone (VI-1) (113 mg, 0.345 mmol) dissolved in dry pyridine (3 ml) was treated with acetyl chloride (44 μl, 0.608 mmol) for 6 h. After work-up the crude product was purified by column chromatography (eluent CH/EtOAc 1:1) to yield the ester (66 mg, 51%).

LC-MS (ES+): rt 5.53 min, m/z (rel. Intens) 371 [(M+H)⁺, 100%]

¹H NMR (400 MHz, CDCl₃) δ (ppm) 0.84 (s, 3H); 1.28 (s, 3H), 1.38-1.49 (m, 1H), 1.70-1.94 (m, 6H), 2.02 (s, 3H), 2.13 (s, 3H), 2.20-2.70 (m, 7H), 5.55-5.56 (m, 1H), 5.71 (s, 1H), 6.17-6.22 (m, 2H).

¹³C NMR (100.6 MHz, CDCl₃) δ (ppm) 14.2, 21.5, 22.1, 22.7, 24.9, 31.4, 33.8, 35.3, 35.6, 35.9, 43.1, 43.3, 47.2, 49.7, 63.4, 69.8, 124.6, 127.1, 139.2, 161.4, 169.6, 198.9, 208.2.

### Propionic acid dydrogesterone-11β-yl ester (Compound No. 17)

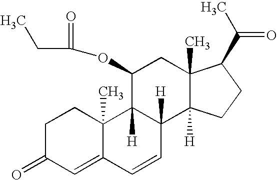

According to the General Procedure D, 11β-hydroxydydrogesterone (VI-1) (150 mg, 0.457 mmol) dissolved in dry pyridine (2 ml) was treated with propanoyl chloride (80 μL, 0.912 mmol) for 8 h. After work-up the crude product was purified by column chromatography (eluent CH/EtOAc 1:1) to yield the ester (75 mg, 43%).

LC-MS (ES+): rt 5.91 min, m/z (rel. Intens) 385 [(M+H)⁺, 100%]

¹³C NMR (100.6 MHz, CDCl₃) δ (ppm) 9.0, 14.3, 22.1, 22.7, 24.8, 28.1, 31.4, 33.8, 35.2, 35.6, 35.9, 43.1, 43.4, 47.2, 49.7, 63.4, 69.6, 124.5, 127.1, 139.3, 161.4, 172.9, 198.9, 208.2.

### Methoxy-acetic acid dydrogesterone-11β-yl ester (Compound No. 18)

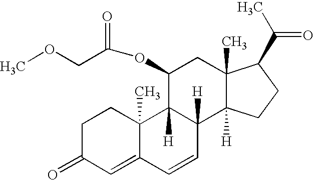

According to the General Procedure D, 11-hydroxydydrogesterone (VI-1) (150 mg, 0.457 mmol) dissolved in dry pyridine (2 ml) was treated with methoxyacetyl chloride (84 μL, 0.914 mmol) for 2.5 h. After work-up the crude product was purified by column chromatography (eluent CH/EtOAc 2:1 to 1.5:1) to yield the ester (135 mg, 74%).

LC-MS (ES+): rt 5.34 min, m/z (rel. Intens) 401 [(M+H)⁺, 100%]

¹³C NMR (100.6 MHz, CDCl₃) δ (ppm) 14.1, 22.0, 22.5, 24.7, 31.2, 33.6, 35.1, 35.4, 35.8, 42.9, 43.2, 47.1, 49.5, 59.3, 63.2, 69.9, 70.4, 124.5, 127.0, 138.9, 161.0, 168.8, 198.6, 208.0.

### Benzoic acid dydrogesterone-11β-yl ester (Compound No. 19)

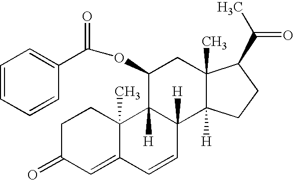

According to the General Procedure D, 11β-hydroxydydrogesterone (VI-1) (328 mg, 1.00 mmol) dissolved in dry pyridine (4 ml) was treated with benzoyl chloride (562 mg, 4.00 mmol) for 3 h. After work-up the crude product was purified by column chromatography (eluent CH/EtOAc 3:1) to yield the ester (335 mg, 77%).

LC-MS (ES+): rt 6.42 min, m/z (rel. Intens) 433 [(M+H)⁺, 100%]

¹³C NMR (100.6 MHz, CDCl₃) δ (ppm) 14.7, 22.2, 22.7, 24.9, 31.4, 33.8, 35.3, 35.7, 36.1, 43.1, 43.6, 47.3, 49.8, 63.4, 70.5, 124.7, 127.2, 128.5, 129.6, 130.3, 133.1, 139.1, 161.3, 165.3, 198.9, 208.1.

m.p. 162-166° C.

[α]_{D}²⁰=−195.2 (c=0.788, MeOH)

### 2-Methoxy-benzoic acid dydrogesterone-11β-yl ester (Compound No. 20)

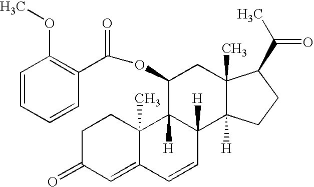

According to the General Procedure D, 11β-hydroxydydrogesterone (VI-1) (82 mg, 0.250 mmol) dissolved in dry pyridine (2 ml) was treated with 2-methoxybenzoyl chloride (80 μl, 0.535 mmol) for 3 h. After work-up the crude product was purified by column chromatography (eluent CH/ethyl acetate 2:1 to 1:1) to yield the ester (63 mg, 54%).

LC-MS (ES+): rt 6.10 min, m/z (rel. Intens) 463 [(M+H)⁺, 100%]

¹H NMR (400 MHz, CDCl₃) δ (ppm) 0.93 (s, 3H), 1.42-1.52 (m, 1H), 1.71-2.10 (m, 5H), 2.14 (s, 3H), 2.22-2.32 (m, 1H), 2.47-2.64 (m, 5H), 2.72-2.78 (m, 1H), 3.87 (s, 3H), 5.71 (s, 1H), 5.82-5.83 (m, 1H), 6.18-6.24 (m, 2H), 6.94-6.98 (m, 2H), 7.44-7.49 (m, 1H), 7.74-7.76 (m, 1H).

¹³C NMR (100.6 MHz, CDCl₃) δ (ppm) 12.5, 20.4, 20.8, 23.0, 29.6, 32.0, 33.5, 33.9, 34.2, 41.4, 41.7, 45.5, 47.9, 53.9, 61.6, 68.3, 110.2, 118.3, 122.7, 125.3, 129.8, 131.9, 137.5, 157.5, 159.8, 163.1, 197.3, 206.5.

### 3-Methoxy-benzoic acid dydrogesterone-11β-yl ester (Compound No. 21)

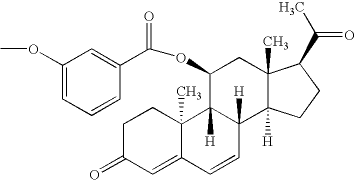

According to the General Procedure D, 11β-hydroxydydrogesterone (VI-1) (100 mg, 0.305 mmol) dissolved in dry pyridine (2 ml) was treated with 3-methoxybenzoyl chloride (182 μl, 1.22 mol) for 48 h. After work-up the crude product was purified by column chromatography (eluent CH/EtOAc) to yield the ester (38 mg, 27%).

LC-MS (ES+): rt 6.37 min, m/z (rel. Intens) 463 [(M+H)⁺, 100%]

¹³C NMR (100.6 MHz, CDCl₃) δ (ppm) 14.7, 22.2, 22.6, 24.8, 31.4, 33.7, 35.6, 36.0, 43.1, 43.5, 47.5, 47.1, 49.6, 55.3, 63.3, 70.5, 114.1, 119.5, 121.8, 124.5, 127.2, 129.5, 131.4, 139.1, 159.5, 161.4, 165.1, 199.1, 208.3.

### 4-Methoxy-benzoic acid dydrogesterone-11β-yl ester (Compound No. 22)

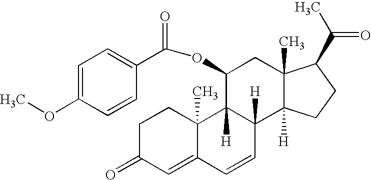

According to the General Procedure D, 11β-hydroxydydrogesterone (VI-1) (100 mg, 0.305 mmol) dissolved in dry pyridine (2 ml) was treated with 4-methoxybenzoyl chloride (100 μl, 0.739 mmol) for 3 h. After work-up the crude product was purified by column chromatography (eluent CH/EtOAc) to yield the ester (19 mg, 13%).

LC-MS (ES+): rt 6.32 min, m/z (rel. Intens) 463 [(M+H)⁺, 100%]

¹H NMR (400 MHz, CDCl₃) δ (ppm) 0.97 (s, 3H), 1.33 (s, 3H), 1.40-1.52 (m, 1H), 1.75-2.02 (m, 5H), 2.07-2.09 (m, 1H), 2.13 (s, 3H), 2.23-2.32 (m, 1H), 2.45-2.64 (m, 5H), 2.75-2.80 (m, 1H), 3.85 (s, 3H), 5.71 (s, 1H), 5.81 (broad s, 1H), 6.18-6.24 (m, 2H), 6.89-6.93 (m, 2H), 7.92-7.95 (m, 2H).

¹³C NMR (100.6 MHz, CDCl₃) δ (ppm) 14.6, 22.1, 22.6, 24.8, 31.3, 33.7, 35.2, 35.6, 36.0, 43.1, 43.5, 47.1, 49.7, 55.4, 63.3, 69.9, 113.7, 122.5, 124.5, 127.1, 131.5, 139.0, 161.3, 163.5, 164.9, 198.9, 208.1.

### Benzoic acid 17-ethoxy-dydrogesterone-11β-yl ester (Compound No. 23)

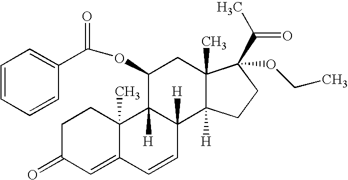

According to the General Procedure D, 17-Ethoxy-11β-hydroxydydrogesterone (VI-5) (45 mg, 0.121 mmol) dissolved in dry pyridine (1 ml) was treated with benzoyl chloride (25 μl, 0.215 mmol) for 2 h. After work-up the crude product was purified by column chromatography (eluent CH/EtOAc 4:1) to yield the ester (35 mg, 61%).

LC-MS (ES+): rt 7.16 min, m/z (rel. Intens) 477 [(M+H)⁺, 100%]

¹H NMR (400 MHz, CDCl₃) δ (ppm) 0.92 (s, 3H), 1.18 (t, J=6.9 Hz, 3H), 1.38 (s, 3H), 1.45-1.53 (m, 1H), 1.75-2.06 (m, 5H), 2.12 (s, 3H), 2.50-2.69 (m, 6H), 2.77-2.83 (m, 1H), 3.00-3.07 (m, 1H), 3.42-3.50 (m, 1H), 5.71 (s, 1H), 5.88-5.89 (m, 1H), 6.18-6.24 (m 2H), 7.40-7.44 (m, 2H), 7.55-7.57 (m, 1H), 7.96-7.99 (m, 2H).

¹³C NMR (100.6 MHz, CDCl₃) δ (ppm) 15.6, 16.2, 21.9, 23.4, 24.3, 26.5, 33.8, 35.4, 35.6, 35.6, 36.0, 44.5, 46.8, 47.4, 60.0, 71.4, 95.6, 124.5, 127.3, 128.5, 129.5, 130.4, 133.1, 139.6, 161.9, 165.4, 199.1, 210.1.

### (3,4,5-Trimethoxy-phenyl)-carbamic acid dydrogesterone-11β-yl ester (Compound No. 24)

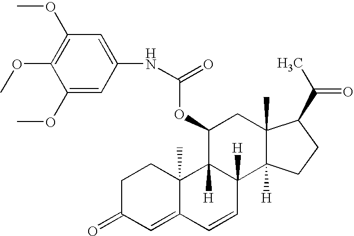

According to the General Procedure C-2, 11β-hydroxydydrogesterone (VI-1) (700 mg, 2.1 mmol) dissolved in DCM 100 ml was stirred with triphosgene (0.39 mg, 1.3 mmol) and DMAP 1 g for 50 min. Then Trimethoxyaniline (1.15 g, 6.3 mmol) and DMAP 1 g were added and the reaction mixture was stirred at RT over night. After work-up the crude product was purified by column chromatography (eluent CH/Eto Ac 2:1) to yield the carbamate (400 mg, 40%).

LC-MS (ES+): rt 5.67 min, m/z (rel. Intens) 538 [(M+H)⁺, 100%]

¹H NMR (500 MHz, CDCl₃) δ (ppm) 0.83 (s, 3H), 1.29 (s, 3H), 1.39-1.47 (m, 1H), 1.72-1.98 (m, 5H), 2.02-2.05 (m, 1H), 2.15 (s, 3H), 2.21-2.29 (m, 1H), 2.43-2.67 (m, 6H), 3.80 (s, 3H), 3.84 (s, 6H), 5.54 (broad s, 1H), 5.72 (s, 1H), 6.19 (s, 2H), 6.55 (s, 1H), 6.67 (s, 2H).

¹³C NMR (125.9 MHz, CDCl₃) δ (ppm) 14.4, 22.1, 22.6, 24.9, 31.4, 33.8, 35.3, 35.6, 35.9, 43.1, 43.6, 47.3, 49.7, 56.2 (2C), 61.0, 63.4, 70.9, 96.7 (2C), 110.6, 124.6, 127.2, 133.7, 139.2, 152.5, 153.5 (2C), 161.5, 199.0, 208.2.

m.p. 126-127° C.

[α]_{D}²⁰=−172.7 (c=0.11, MeOH)

### (1H-Indazol-6-yl)-carbamic acid dydrogesterone-11β-yl ester (Compound No. 25)

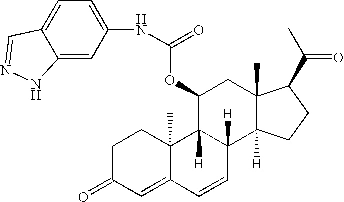

According to the General Procedure C-2, 11β-hydroxydydrogesterone (VI-1) (700 mg, 2.1 mmol) dissolved in 100 ml DCM was stirred with triphosgene (0.39 mg, 1.3 mmol) and DMAP 1 g for 50 min. Then 6-Aminoindazole (0.84 g, 6.3 mmol) and DMAP 1 g were added and the reaction mixture was stirred at RT over night. After work-up the crude product was purified by column chromatography (eluent CH/EtOAc 2:1) and preparative reverse phase HPLC yielding 250 mg of the desired carbamate (25%).

LC-MS (ES+): rt 5.27 min, m/z (rel. Intens) 488 [(M+H)⁺, 100%]

¹H NMR (501 MHz, DMSO-d₆) δ ppm 0.8 (s, 3H) 1.3 (s, 3H) 1.3-1.5 (m, 1H) 1.6-1.7 (m, 1H) 1.8-1.9 (m, 3H) 1.9-2.0 (m, 2H) 2.0-2.2 (m, 4H) 2.3-2.4 (m, 3H) 2.5-2.7 (m, 3H) 5.4-5.5 (m, 1H) 5.7 (s, 1H) 6.3 (s, 2H) 7.1 (dd, J=8.7, 1.7 Hz, 1H) 7.6 (d, J=8.9 Hz, 1H) 7.9 (s, 1H) 7.9 (s, 1H) 9.5 (s, 1H) 12.8 (s, 1H)

¹³C NMR (126 MHz, DMSO-d₆) δ ppm 14.2, 21.6, 21.9, 24.2, 30.9, 33.4, 34.7, 35.1, 35.3, 42.4, 42.4, 47.0, 48.7, 62.1, 69.9, 97.6, 113.6, 118.6, 120.4, 123.5, 126.5, 133.1, 137.4, 139.6, 140.4, 152.6, 161.5, 197.7, 208.1

### (3-Trifluoromethoxy-phenyl)-carbamic acid dydrogesterone-11β-yl ester (Compound No. 26)

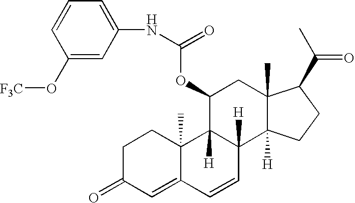

According to the General Procedure C-2, 11β-hydroxydydrogesterone (VI-1) (850 mg, 2.6 mmol) dissolved in 100 ml DCM was stirred with triphosgene (0.42 mg, 1.4 mmol) and DMAP (1 g) for 50 min. Then 3-Trifluormethoxyaniline (1.38 g, 7.8 mmol) and DMAP (1.5 g) were added and the reaction mixture was stirred at RT over night. After work-up the crude product was purified by column chromatography (eluent CH/EtOAc 2:1) and recrystallized to yield the carbamate (780 mg, 56%).

LC-MS (ES+): rt 6.61 min, m/z (rel. Intens) 532 [(M+H)⁺, 100%]

¹H NMR (501 MHz, DMSO-d₆) δ ppm 0.81 (s, 3H) 1.26 (s, 3H) 1.32-1.43 (m, 1H) 1.60-1.73 (m, 1H) 1.75-1.88 (m, 3H) 1.89-1.97 (m, 2H) 2.04-2.13 (m, 4H) 2.28-2.40 (m, 3H) 2.53-2.68 (m, 3H) 5.46 (s, 1H) 5.66 (s, 1H) 6.26 (d, 2H) 6.96 (d, 1H) 7.39 (t, 1H) 7.45 (d, 1H) 7.58 (s, 1H) 9.70 (s, 1H)

¹³C NMR (126 MHz, DMSO-d₆) δ ppm 14.1, 21.6, 22.0, 24.2, 30.9, 33.4, 34.6, 35.1, 35.3, 42.4, 42.4, 46.9, 48.7, 62.1, 70.3, 110.3, 114.2, 116.9, 120.0, 123.6, 126.5, 130.3, 139.5, 140.9, 148.6, 152.4, 161.4, 197.8, 208.1

### (4-Trifluoromethoxy-phenyl)-carbamic acid dydrogesterone-11β-yl ester (Compound No. 27)

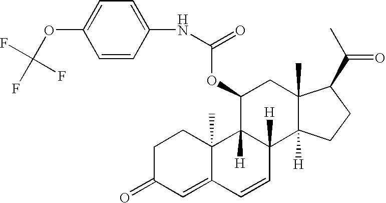

According to the General Procedure C-2, 11β-hydroxydydrogesterone (VI-1) (850 mg, 2.6 mmol) dissolved in 100 ml DCM was stirred with triphosgene (0.42 mg, 1.4 mmol) and DMAP (1 g) for 50 min. Then 4-Fluormethoxyaniline (1.38 g, 7.8 mmol) and DMAP (1.5 g) were added and the reaction mixture was stirred at RT over night. After work-up the crude product was purified by column chromatography (eluent CH/EtOAc 2:1) to yield the carbamate (650 mg, 47%).

LC-MS (ES+): rt 6.59 min, m/z (rel. Intens) 532 [(M+H)⁺, 100%]

¹H NMR (501 MHz, CDCl₃) δ ppm 0.85 (s, 3H) 1.29 (s, 3H) 1.37-1.49 (m, 1H) 1.71-2.00 (m, 5H) 2.04 (d, 1H) 2.14 (s, 3H) 2.20-2.30 (m, 1H) 2.42-2.62 (m, 5H) 2.66 (dd, 1H) 5.55 (s, 1H) 5.71 (s, 1H) 6.17-6.22 (m, 2H) 6.67 (s, 2H) 7.16 (d, 2H) 7.41 (d, 2 H)

¹³C NMR (126 MHz, CDCl₃) δ ppm 14.4, 22.2, 22.7, 24.9, 31.4, 33.8, 35.3, 35.6, 35.9, 43.1, 43.6, 47.3, 49.7, 63.4, 71.3, 120.5, 119.9, 121.9, 124.6, 127.2, 136.4, 139.1, 145.0, 152.4, 161.4, 199.0, 208.2

m.p. 273-275° C.

### Pyridin-2-yl-carbamic acid dydrogesterone-11β-yl ester (Compound No. 28)

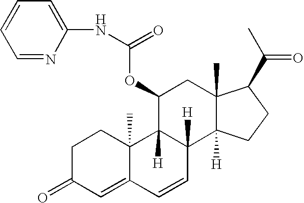

According to the General Procedure C-2, 11β-hydroxydydrogesterone (VI-1) (850 mg, 2.6 mmol) dissolved in 100 ml DCM was stirred with triphosgene (0.42 mg, 1.4 mmol) and DMAP (1 g) for 50 min. Then 2-Aminopyridine (0.73 g, 7.8 mmol) and DMAP (1.5 g) were added and the reaction mixture was stirred at RT over night. After work-up the crude product was purified by column chromatography (eluent CH/EtOAc 2.5:1) to yield the carbamate (800 mg, 69%).

LC-MS (ES+): rt 5.51 min, m/z (rel. Intens) 449 [(M+H)⁺, 100%]

m.p. 228-230° C.

¹H NMR (501 MHz, CDCl₃) δ ppm 0.84 (s, 3H) 1.30 (s, 3H) 1.39-1.49 (m, 1H) 1.74-1.93 (m, 4H) 1.94-2.02 (m, 1H) 2.06 (d, 1H) 2.14 (s, 3H) 2.20-2.30 (m, 1H) 2.42-2.63 (m, 5H) 2.68 (dd, 1H) 5.54-5.59 (m, 1H) 5.72 (s, 1H) 6.20 (m, 2H) 6.99 (dd, 1H) 7.56 (s, 1H) 7.66-7.72 (m, 1H) 7.94 (d, 1H) 8.23 (d, 1H)

¹³C NMR (126 MHz, CDCl₃) δ ppm 14.3, 22.2, 22.6, 24.8, 31.3, 33.8, 35.3, 35.5, 35.9, 43.1, 43.6, 47.2, 49.7, 63.4, 71.2, 112.4, 119.1, 124.6, 127.2, 138.4, 139.1, 147.9, 151.3, 152.0, 161.3, 198.9, 208.1

### (3-Methoxy-phenyl)-carbamic acid (17α-ethoxy-1,2-methylene-dydrogesterone-11β-yl ester (Compound No. 29)

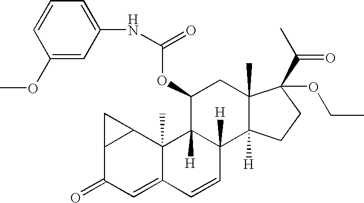

According to General Procedure C-2, 17α-Ethoxy-11β-hydroxy-1,2-methylen-dydrogesterone (VI-6) (120 mg, 0.3 mmol) dissolved in 15 ml DCM was stirred with triphosgene (47 mg, 0.16 mmol) and DMAP (100 mg) for 50 min. Then 3-Methoxyaniline (0.1 ml, 0.9 mmol) and DMAP (100 mg) were added and the reaction mixture was stirred at RT over night. After work-up the crude product was purified by column chromatography (eluent DCM/EtOAc 95:5) to yield the carbamate (85 mg, 53%).

LC-MS (ES+): rt 6.64 min, m/z (rel. Intens) 534 [(M+H)⁺, 100%]

¹H NMR (501 MHz, CDCl₃) δ ppm 0.80 (s, 3H) 0.82-0.87 (m, 1H) 1.16 (t, 3H) 1.35-1.48 (m, 5H) 1.72-1.86 (m, 2H) 1.91 (d, 1H) 1.96-2.03 (m, 1H), 2.04-2.09 (m, 1H) 2.14 (s, 3H) 2.21 (d, 1H) 2.44-2.57 (m, 2H) 2.60-2.71 (m, 2H) 2.99-3.06 (m, 1H) 3.41-3.48 (m, 1H) 3.80 (s, 3H) 5.52 (s, 1H) 5.81-5.85 (m, 1H) 6.03-6.13 (m, 2H) 6.52 (s, 1H) 6.62 (dd, 1H) 6.87 (dd, 1H) 7.10 (s, 1H) 7.19 (t, 1H)

¹³C NMR (126 MHz, CDCl₃) δ ppm 14.4, 15.6, 15.9, 23.4, 24.2, 25.8, 26.5 26.9, 28.8, 35.3, 35.7, 37.0, 44.1, 45.4, 47.3, 55.3, 60.0, 72.4, 95.6, 104.7, 109.3, 110.9, 120.7, 127.4, 129.8, 138.0, 139.0, 152.2, 155.4, 160.4, 198.4, 210.3

### Benzo[1,3]-dioxol-5-yl-carbamic acid 1,2-methylene-dydrogesterone-11β-yl ester (Compound No. 30)

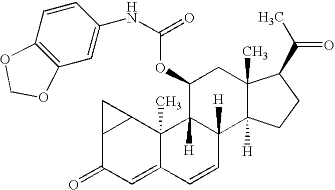

Compound No. 30 was prepared from Compound No. 13 in a two-step synthesis according to the general description part A-I and as displayed in SCHEME I, and as described in detail below:

### Intermediate: Benzo[1,3]dioxol-5-yl-carbamic acid 1,2-unsaturated dydrogesterone-11β-yl ester

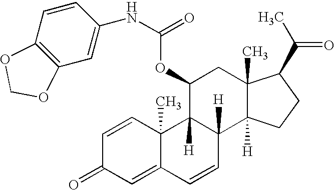

To a solution of the carbamate (Compound No. 13) (100 mg, 0.2 mmol) in 2 ml dry dioxane (containing 1 mg HCl/ml), was added 2,3-dichloro-5,6-dicyano-p-benzochinone (69 mg, 0.3 mmol). The mixture was stirred at ambient temperature for 90 min. Then 40 mg CaCO₃ were added and the reaction mixture was stirred at ambient temperature for 20 min. Subsequently to filtration the mixture was refluxed for 90 min. After dilution with DEE the organic phase was washed successively with 1M aq KHSO₄ solution, 1M aq NaHCO₃ solution and brine, dried over Na₂SO₄, filtered and concentrated in vacuo. The crude product was used without further purification (50 mg, 50%). LC-MS (ES+): rt 5.75 min, m/z (rel Intens) 490 [(M+H)⁺, 100%]

Product: A solution of trimethylsulfoxonium iodide (91 mg, 0.4 mmol) in DMSO (0.5 ml) was stirred for 60 min at 60° C. with 11 mg NaH (4.6 mmol). To this in situ prepared solution of dimethylsulfoxonium methylide a solution of the 1,2-unsaturated dydrogesterone-11β-yl ester (50 mg, 0.1 mmol) in 0.5 ml THF was added. After stirring for 4 h, the mixture stood over night at ambient temperature. The mixture was quenched with water and concentrated in vacuo. The residue was diluted with EtOAc. The organic phase was with brine, dried over Na₂SO₄, filtered and concentrated in vacuo. The crude product was purified by reversed phase preparative HPLC yielding 5 mg of the desired product (5%).

LC-MS (ES+): rt 5.81 min, m/z (rel Intens) 504 [(M+H)⁺, 100%]

¹H NMR (500 MHz, CDCl₃) δ (ppm) 0.85 (m, 4H), 1.33-1.39 (m, 2H), 1.40 (s, 3H), 1.70-1.93 (m, 4H), 2.00 (m, 1H), 2.07 (q, 1H), 2.15 (s, 3H), 2.20-2.29 (m, 2H), 2.45 (d, 1H), 2.55 (t, 1H), 2.61-2.70 (m, 1H), 5.53 (s, 1H), 5.75-5.79 (m, 1H), 5.94 (s, 2H), 6.06 (dd, 1H), 6.10 (d, 1H), 6.35 (s, 1H) 6.67 (dd, 1H), 6.73 (d, 1H), 7.05 (s, 1H).

### (3-Formyl-phenyl)-carbamic acid dydrogesterone-11β-yl ester (Compound No. 31)

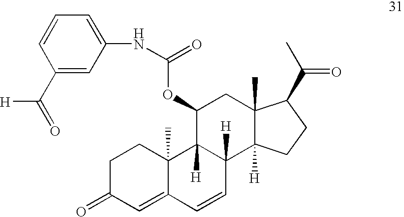

Compound No. 31 was prepared in a two step synthesis from 11β-Hydroxy-dydrogesterone (VI).

In a first step, the intermediate was prepared as described in general procedure C-2 (according to SCHEME VII) using 3-[1,3]Dioxolan-2-yl-aniline as amine building block. The following intermediate was obtained, which was used without further purifications in the next step:

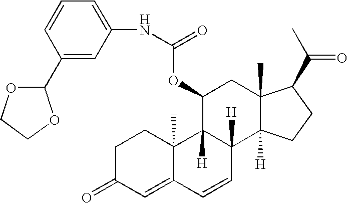

LC-MS (ES+): rt 5.76 min, m/z (rel Intens) 520 [(M+H)+, 100%]

Product: 227 mg of the intermediate steroid were dissolved in 23 ml acetone. 22 mg pTosOH, 2 ml H₂O and 1 ml 1 M H₂SO₄ were added. The reaction mixture was stirred for 5.5 h. The reaction mixture was than adjusted to pH 8 with 1 M NaHCO₃ solution. Acetone was removed by evaporation. The residue was dissolved in DCM and washed twice with water. After evaporation 147 mg were obtained which were further purified by flash chromatography (EtOAc/CH 1:1 to 99:1) yielding 120 mg compound No. 31 (68% overall yield).

LC-MS (ES+): rt 5.75 min, m/z (rel Intens) 476 [(M+H)⁺, 100%]

### [3-(Hydroxyimino-methyl)-phenyl]-carbamic acid dydrogesterone-11-yl ester (Compound No. 32)

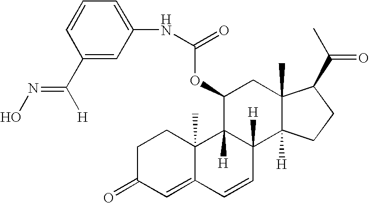

Compound No. 32 was prepared starting from compound No. 31 according to the general description part D-V and as described in more detail below.

115 mg of compound No. 31, 3 ml pyridine and 19 mg Hydroxylamine*HCl were mixed and stirred for 5 hours at ambient temperature. The reaction mixture was poured on ice and extracted 3 times with DCM. The organic layer was washed with water, 1 M KHSO₄ solution and dried over Na₂SO₄. The material was further purified using reverse phase prep. HPLC yielding 21 mg of compound No. 32.

LC-MS (ES+): rt 5.48 min, m/z (rel Intens) 491 [(M+H)⁺, 100%]

### [3-(N-Ethylcarbamoyl-oximino-formyl)-phenyl]-carbamic acid dydrogesterone-11β-yl ester (Compound No. 33)

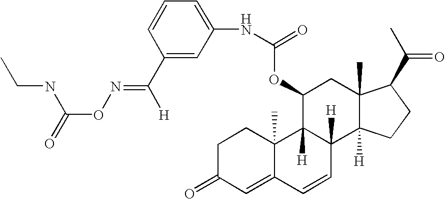

Compound No. 33 was prepared starting from compound No. 32 according to the general description part D-V: 19 mg of compound No. 32, 5 ml ACN, 4 μl TEA, and 8 μl ethylisocyanate were mixed and stirred for 48 hours at ambient temperature. The mixture was evaporated and than purified by flash chormatogrphy yielding 12 mg of the desired compound No. 33.

LC-MS (ES+): rt 5.68 min, m/z (rel Intens) 562 [(M+H)+, 100%]

### Benzo[1,3]-dioxol-5-yl-carbamic acid 17-ethoxy-dydrogesterone-11β-yl este (Compound No. 34)

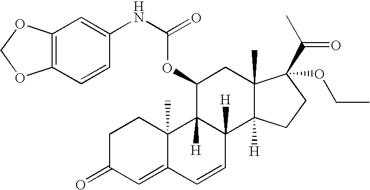

According to the General Procedure B, 17-Ethoxy-dydrogesterone-11β-yl 4-nitrophenyl carbonate (VIII-2) dissolved in DCM was treated with 3,4-methylendioxyaniline, pyridine and DMAP (cat). After complete reaction, the mixture was worked-up and the crude product was purified by column chromatography to yield the desired carbamate.

LC-MS (ES+): rt 6.58 min, m/z (rel Intens) 536 [(M+H)+, 100%]

### (4-Dimethylamino-phenyl)-carbamic acid dydrogesterone-11β-yl ester (Compound No. 35)

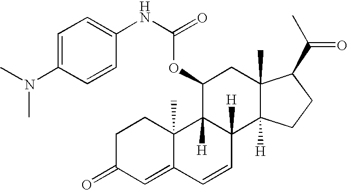

According to the General Procedure C-1, 11β-Hydroxy-dydrogesterone (VI-1) was reacted with 3-(N,N-dimethyl-amino)-aniline to yield the respective carbamate.

LC-MS (ES+): rt 5.93 min, m/z (rel Intens) 491 [(M+H)+, 100%]

Further examples of compounds of the invention were prepared by library synthesis. The library synthesis was generally performed according to General Procedure C-2 (according to SCHEME VII): To a solution of the 0.05 mmol 11β-Hydroxy-dydrogesterone (VI) in 4 ml dry DCM was added DMAP (0.39 mmol, 7.9 eq) and triphosgene (0.025 mmol, 0.5 eq). The mixture was stirred at RT for 30 min. Then the respective amine R5-NH₂ (0.15 mmol, 3 eq) and DMAP (40 mg) were added and the reaction mixture was stirred for 48 h at RT. After reaction, the organic phase was washed successively with H₂O (4 ml), 1M aq KHSO₄ solution (4 ml), and 1M aq NaCl solution (4 ml). Control of the reaction was performed by TLC analysis (EtOAc/CH 2:1). The organic phase was dried over Na₂SO₄, filtered and concentrated in vacuo. The crude product was analysed by LC-MS.

Illustrative compounds falling under the scope of formula (IIIb) with R1, R2 and R3=H, prepared by library synthesis, are listed in the following table 1:

TABLE 1

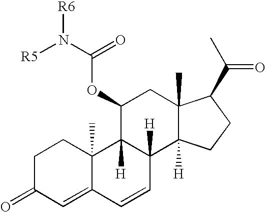

| | | R5 | | | |
|---|---|---|---|---|---|
| CpdNo. | NAME | 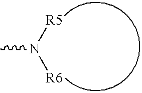 | R6 | MS(m/z) | Rt(min) |
| 36 | (3,5-Bis-trifluoromethyl-phenyl)-carbamic acid dydrogesterone-11-yl ester | 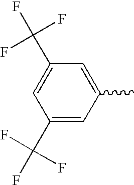 | H | 583 | 7.03 |
| 37 | (1,5-Dimethyl-3-oxo-2-phenyl-2,3-dihydro-1H-pyrazol-4-yl)-carbamic acid dydrogesterone-11-yl ester | 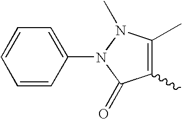 | H | 557 | 4.94 |
| 38 | (5-Methyl-isoxazol-3-yl)-carbamic acid dydrogesterone-11-yl ester | 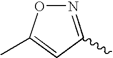 | H | 453 | 5.40 |
| 39 | [2-(dydrogesterone-11-yloxycarbonylamino)-thiazol-4-yl]-acetic acid ethyl ester | 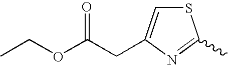 | H | 540 | 5.63 |
| 40 | [2-(1H-Indol-3-yl)-ethyl]-carbamic acid dydrogesterone-11-yl ester | 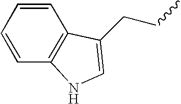 | H | 514 | 5.80 |
| 41 | (1H-Indazol-6-yl)-carbamic aciddydrogesterone-11-yl ester | 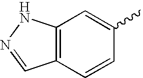 | H | 487 | 5.28 |
| 42 | Benzo[1,3]dioxol-5-ylmethyl-carbamic acid dydrogesterone-11-yl ester | 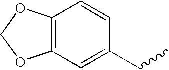 | H | 505 | 5.71 |
| 43 | 4-(2-Methoxy-phenyl)-piperazine-1-carboxylic aciddydrogesterone-11-yl ester | 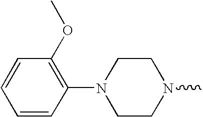 | | 546 | 546 |
| 44 | 4-(3-Trifluoromethyl-phenyl)-piperazine-1-carboxylic aciddydrogesterone-11-yl ester | 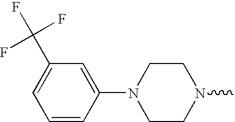 | | 585 | 585 |
| 45 | (4-Morpholin-4-yl-phenyl)-carbamic acid dydrogesterone-11-yl ester | 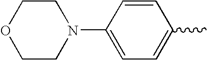 | H | 532 | 5.61 |
| 46 | (2-Morpholin-4-yl-ethyl)-carbamic acid dydrogesterone-11-yl ester | 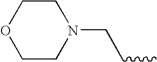 | H | 484 | 4.38 |
| 47 | Pyridin-2-ylmethyl-carbamic aciddydrogesterone-11-yl ester | 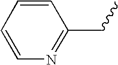 | H | 462 | 4.96 |
| 48 | (2-Pyridin-2-yl-ethyl)-carbamicacid dydrogesterone-11-yl ester | 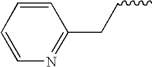 | H | 476 | 4.97 |
| 49 | Pyridin-3-ylmethyl-carbamic aciddydrogesterone-11-yl ester | 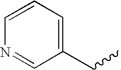 | H | 462 | 4.85 |
| 50 | Pyridin-4-ylmethyl-carbamic aciddydrogesterone-11-yl ester | 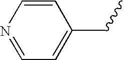 | H | 462 | 4.78 |
| 51 | Quinolin-3-yl-carbamic aciddydrogesterone-11-yl ester | 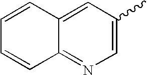 | H | 498 | 5.70 |
| 52 | Pentafluorophenyl-carbamic aciddydrogesterone-11-yl ester | 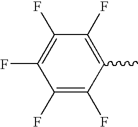 | H | 537 | 6.13 |
| 53 | (3,4-Dimethoxy-benzyl)-carbamic acid dydrogesterone-11-yl ester | 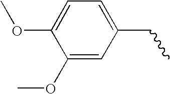 | H | 521 | 5.45 |
| 54 | (3-Imidazol-1-yl-propyl)-carbamic acid dydrogesterone-11-yl ester | 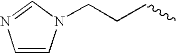 | H | 479 | 7.39 |
| 55 | (4-Methoxy-benzyl)-carbamicacid dydrogesterone-11-yl ester | 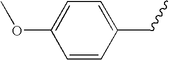 | H | 491 | 5.77 |
| 56 | [2-(3-Methoxy-phenyl)-ethyl]-carbamic acid dydrogesterone-11-yl ester | 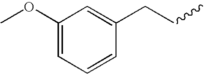 | H | 505 | 5.88 |
| 57 | [2-(3,4-Dimethoxy-phenyl)-ethyl]-carbamic aciddydrogesterone-11-yl ester | 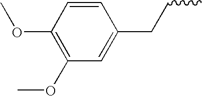 | H | 535 | 5.52 |
| 58 | [2-(4-Fluoro-phenyl)-ethyl]-carbamic acid dydrogesterone-11-yl ester | 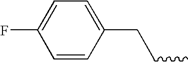 | H | 493 | 5.96 |
| 59 | [2-(4-Methoxy-phenyl)-ethyl]-carbamic acid dydrogesterone-11-yl ester | 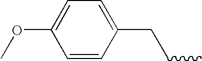 | H | 505 | 5.87 |
| 60 | (2-Oxo-tetrahydro-furan-3-yl)-carbamic acid dydrogesterone-11-yl ester | 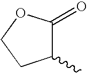 | H | 455 | 4.91 |
| 61 | (2-Oxo-2-phenyl-ethyl)-carbamicacid dydrogesterone-11-yl ester | 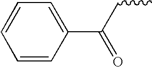 | H | 489 | 5.66 |
| 62 | [2-(4-Sulfamoyl-phenyl)-ethyl]-carbamic acid dydrogesterone-11-yl ester | 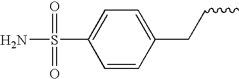 | H | 554 | 5.08 |
| 63 | (3-Trifluoromethoxy-phenyl)-carbamic acid dydrogesterone-11-yl ester | 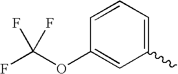 | H | 531 | 6.70 |
| 64 | [2-(7-Methyl-1H-indol-3-yl)-ethyl]-carbamic aciddydrogesterone-11-yl ester | 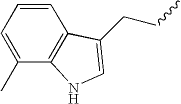 | H | 528 | 6.00 |
| 65 | (4-Fluoro-3-trifluoromethyl-benzyl)-carbamic aciddydrogesterone-11-yl ester | 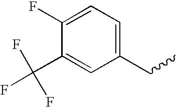 | H | 547 | 6.22 |
| 66 | (5-Phenyl-1H-pyrazol-3-yl)-carbamic acid dydrogesterone-11-yl ester | 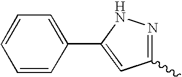 | | 514 | 5.57 |
| 67 | (4-Methanesulfonyl-benzyl)-carbamic acid dydrogesterone-11-yl ester | 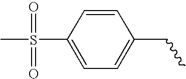 | H | 539 | 5.18 |
| 68 | (4-Morpholin-4-yl-benzyl)-carbamic acid dydrogesterone-11-yl ester | 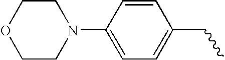 | H | 546 | 5.53 |
| 69 | Quinolin-6-yl-carbamic aciddydrogesterone-11-yl ester | 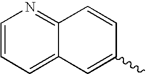 | H | 498 | 5.50 |
| 70 | (2,3-Dihydro-benzo[1,4]dioxin-6-yl)-carbamic aciddydrogesterone-11-yl ester | 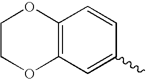 | H | 505 | 5.79 |
| 71 | (3-Cyano-phenyl)-carbamic aciddydrogesterone-11-yl ester | 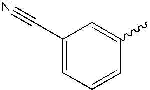 | H | 472 | 5.89 |
| 72 | (3,4-Difluoro-phenyl)-carbamicacid dydrogesterone-11-yl ester | 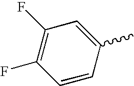 | H | 483 | 6.22 |
| 73 | (3,4-Dichloro-phenyl)-carbamicacid dydrogesterone-11-yl ester | 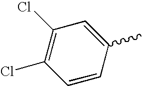 | H | 515 | 6.74 |
| 74 | (3-Trifluoromethoxy-phenyl)-carbamic acid dydrogesterone-11-yl ester | 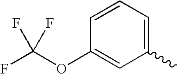 | H | 531 | 6.70 |
| 75 | (4-Difluoromethoxy-phenyl)-carbamic acid dydrogesterone-11-yl ester | 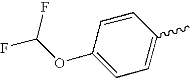 | H | 513 | 6.12 |
| 76 | (3-Bromo-4-methyl-phenyl)-carbamic acid dydrogesterone-11-yl ester | 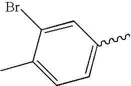 | H | 539 | 6.74 |
| 77 | (2-Methyl-4-oxo-4H-chromen-7-yl)-carbamic aciddydrogesterone-11-yl ester | 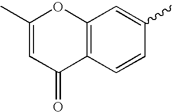 | H | 529 | 5.55 |
| 78 | (3-Fluoro-benzyl)-carbamic aciddydrogesterone-11-yl ester | 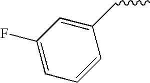 | H | 479 | 5.86 |
| 79 | (2-Thiophen-2-yl-ethyl)-carbamic acid dydrogesterone-11-yl ester | 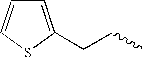 | H | 481 | 5.87 |
| 80 | Cyanomethyl-carbamic aciddydrogesterone-11-yl ester | 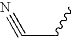 | H | 410 | 5.00 |
| 81 | (3-Acetyl-phenyl)-carbamic aciddydrogesterone-11-yl ester | 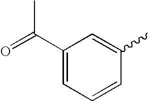 | H | 489 | 5.73 |
| 82 | (4-Acetylamino-phenyl)-carbamicacid dydrogesterone-11-yl ester | 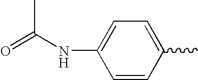 | H | 504 | 3.55 |
| 83 | (3-Oxo-1,3-dihydro-isobenzofuran-5-yl)-carbamicacid dydrogesterone-11-yl ester | 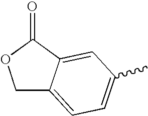 | H | 503 | 3.80 |
| 84 | (5-Methyl-thiazol-2-yl)-carbamicacid dydrogesterone-11-yl ester | 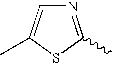 | H | 468 | 3.80 |
| 85 | (3-Cyano-4-fluoro-phenyl)-carbamic acid dydrogesterone-11-yl ester | 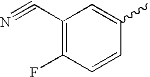 | H | 490 | 4.11 |
| 86 | (1H-Indol-4-yl)-carbamic aciddydrogesterone-11-yl ester | 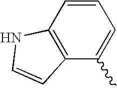 | H | 486 | 3.87 |
| 87 | [3-(Morpholine-4-sulfonyl)-phenyl]-carbamic aciddydrogesterone-11-yl ester | 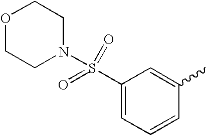 | H | 596 | 3.93 |
| 88 | (1H-Indol-6-yl)-carbamic aciddydrogesterone-11-yl ester | 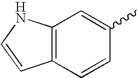 | H | 486 | 3.96 |
| 89 | (3-Methanesulfonylamino-phenyl)-carbamic aciddydrogesterone-11-yl ester | 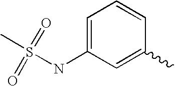 | H | 540 | 3.73 |
| 90 | [4-(4-Methyl-4H-[1,2,4]triazol-3-yl)-phenyl]-carbamic aciddydrogesterone-11-yl ester | 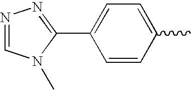 | H | 528 | 3.41 |
| 91 | (1H-Indol-5-yl)-carbamic aciddydrogesterone-11-yl ester | 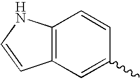 | H | 486 | 5.66 |
| 92 | (3-Methylsulfanyl-phenyl)-carbamic acid dydrogesterone-11-yl ester | 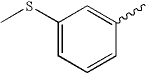 | H | 493 | 6.30 |
| 93 | (4-Hydroxy-phenyl)-carbamicacid dydrogesterone-11-yl ester | 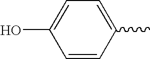 | H | 463 | 5.25 |
| 94 | (4-Methylsulfanyl-phenyl)-carbamic acid dydrogesterone-11-yl ester | 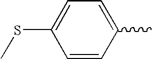 | H | 493 | 6.26 |
| 95 | (3-Acetylamino-phenyl)-carbamicacid dydrogesterone-11-yl ester | 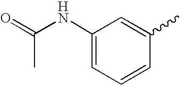 | H | 504 | |
| 96 | (1H-Benzotriazol-5-yl)-carbamicacid dydrogesterone-11-yl ester | 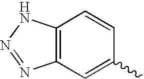 | H | 488 | 5.50 |
| 97 | (5-tert-Butyl-isoxazol-3-yl)-carbamic acid dydrogesterone-11-yl ester | 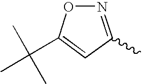 | H | 494 | 6.18 |
| 98 | (1,1-Dioxo-1H-1lambda*6*-benzo[b]thiophen-6-yl)-carbamicacid dydrogesterone-11-yl ester | 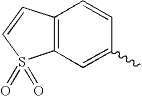 | H | 535 | 5.61 |
| 99 | (1-Methyl-1H-indazol-6-yl)-carbamic acid dydrogesterone-11-yl ester | 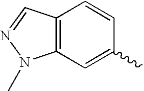 | H | 501 | 5.57 |
| 100 | [3-(2-Oxo-pyrrolidin-1-yl)-phenyl]-carbamic aciddydrogesterone-11-yl ester | 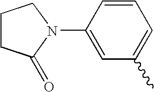 | H | 530 | 5.49 |
| 101 | (4-Methyl-3-oxo-3,4-dihydro-2H-benzo[1,4]oxazin-6-yl)-carbamic acid dydrogesterone-11-yl ester | 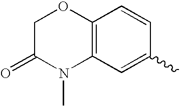 | H | 532 | 5.54 |
| 102 | (2-Methyl-3-oxo-3,4-dihydro-2H-benzo[1,4]thiazin-6-yl)-carbamic acid dydrogesterone-11-yl ester | 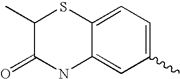 | H | 548 | 5.70 |
| 103 | (3-Pyrrol-1-yl-phenyl)-carbamicacid dydrogesterone-11-yl ester | 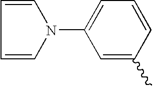 | H | 512 | 6.45 |
| 104 | (3-Methoxy-5-tetrazol-1-yl-phenyl)-carbamic aciddydrogesterone-11-yl ester | 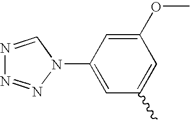 | H | 545 | 5.67 |

### Further Example Compounds Falling Under the Scope of Formula (IVb)

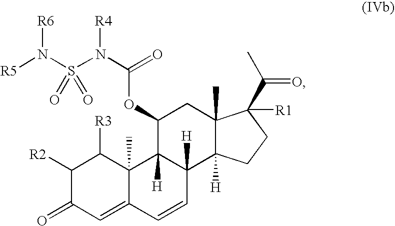 [0523] wherein R1, R2, R3, R4 and R6 each represent hydrogen.

The following compounds were prepared according to the general description part C-IV and as displayed in SCHEME IX, and as described in detail below: First, 0.2 mmol of 11β-Hydroxy-dydrogesterone (VIII) were dissolved in 5 ml DCM. Then, 0.2 mmol chlorosulfonylisocyanate were added at RT and the reaction mixture was stirred for 20 min before the correspondent amine (0.4 mmol) and 0.2 mmol TEA were added. The reaction mixture was stirred over night, diluted with further DCM and washed with 1 M KHSO₄ solution. The organic layer was dried over Na₂SO₄ and evaporated under reduced pressure. If necessary the product was further purified by flash chromatography with EtOAc and CH.

Examples of compounds of falling under the scope of formula (IVb) with R1, R2, R3, R4 and R6=H, are listed in table 2:

TABLE 2

| Cpd | | | MS | Rt |
|---|---|---|---|---|
| No. | FORMULA | R5 Name | (m/z) | (min) |
| 105 | 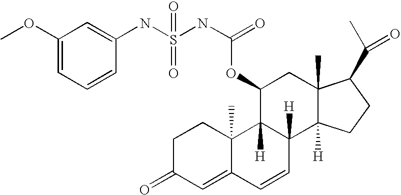 | 3-methoxy-phenyl | 557 | 5.11 |
| 106 | 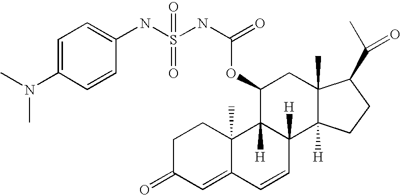 | 4-dimethylamino-phenyl | 570 | 5.37 |
| 107 | 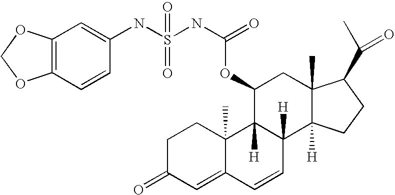 | benzo[1,3]dioxol-5-yl | 571 | 5.07 |
| 108 | 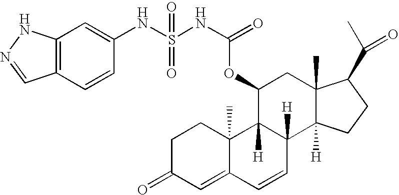 | 1H-indazol-6-yl | 567 | 4.70 |
| 109 | 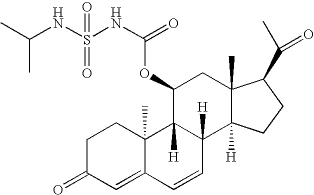 | isopropyl | 493 | 5.15 |
| 110 | 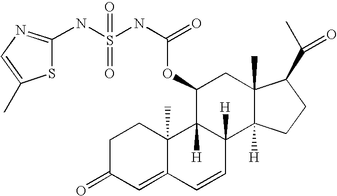 | 5-methyl-thiazol-2-yl | 548 | 4.82 |

### BRIEF DESCRIPTION OF THE DRAWINGS

FIG. 1 is a graph showing the results of the Clauberg-McPhail Assay, and

FIG. 2 is a graph showing the results of the Guinea Pig Model Assay.

### BIOLOGICAL TESTING MATERIALS AND METHODS

### I. Progesterone Receptor Binding Assay

The progesterone receptor (PR) binding assays were performed at CEREP (Celle I'Evescault, France).

### Procedure:

The binding to the human progesterone receptor (Assay Cat No: Progesterone receptor-814h) was be measured using 3H-R5020 as ligand and MCF7 cells as the source of progesterone receptor. The assay is performed as described by Eckert & Katzenellenbogen [1982]. The binding to the bovine progesterone receptor (Assay Cat No: Progesterone receptor-814) was measured using 3H-R5020 as ligand and uterus tissue as the source of progesterone receptor. The assay was performed as described by Hurd & Moudgil [1988]. The assay does not discriminate between the two progesterone receptor isoforms PRα and PRβ.

### Results

The results of the receptor binding assay are presented as the individual pKi values, which were determined by measuring the binding activity to the human or bovine PR, as indicated, for a concentration range of each compound. The data for selected compounds are summarized in the following Table 3:

TABLE 3

| | | Human PR | Bovine PR |
|---|---|---|---|
| Compound | | binding | binding |
| No. | Name | (pKi) | (pKi) |
| 1 | Isopropyl-carbamic acid dydrogesterone-11-yl | | 6.2 |
| | ester | | |
| 2 | Benzyl-carbamic acid dydrogesterone-11-yl ester | | 6.6 |
| 12 | (3-Methoxy-phenyl)-carbamic acid 17-ethoxy- | | 7.6 |
| | dydrogesterone-11-yl ester | | |
| 13 | Benzo[1,3]dioxol-5-yl-carbamic acid | 6.2 | 6.9 |
| | dydrogesterone-11-yl ester | | |
| 14 | (3-Carbamoyl-phenyl)-carbamic acid | | 6.4 |
| | dydrogesterone-11-yl ester | | |
| 15 | (3-Methoxy-phenyl)-carbamic acid | | 6.4 |
| | dydrogesterone-11-yl ester | | |
| 19 | Benzoic acid dydrogesterone-11-yl ester | | 6.9 |
| 21 | 3-Methoxy-benzoic acid dydrogesterone-11-yl | | 6.2 |
| | ester | | |
| 22 | 4-Methoxy-benzoic acid dydrogesterone-11-yl | | 7.4 |
| | ester | | |
| 23 | Benzoic acid 17-ethoxy-dydrogesterone-11-yl | | 6.6 |
| | ester | | |
| 24 | (3,4,5-Trimethoxy-phenyl)-carbamic acid | 6.7 | |
| | dydrogesterone-11-yl ester | | |
| 29 | (3-Methoxy-phenyl)-carbamic acid (17α-ethoxy- | 6.4 | |
| | 1,2-methylene-dydrogesterone-11β-yl ester | | |
| 32 | [3-(Hydroxyimino-methyl)-phenyl]-carbamic acid | 6.5 | |
| | dydrogesterone-11-yl ester | | |
| 34 | Benzo[1,3]dioxol-5-yl-carbamic acid 17-ethoxy- | 6.9 | |
| | dydrogesterone-11-yl ester | | |
| 41 | (1H-Indazol-6-yl)-carbamic acid dydrogesterone- | 6.5 | |
| | 11-yl ester | | |
| 42 | Benzo[1,3]dioxol-5-ylmethyl-carbamic acid | 6.8 | |
| | dydrogesterone-11-yl ester | | |
| 43 | 4-(2-Methoxy-phenyl)-piperazine-1-carboxylic | 6.2 | |
| | acid dydrogesterone-11-yl ester | | |
| 51 | Quinolin-3-yl-carbamic acid dydrogesterone-11-yl | 6.4 | |
| | ester | | |
| 55 | (4-Methoxy-benzyl)-carbamic acid | 7.0 | |
| | dydrogesterone-11-yl ester | | |
| 63 | (3-Trifluoromethoxy-phenyl)-carbamic acid | 6.2 | |
| | dydrogesterone-11-yl ester | | |
| 67 | (4-Methanesulfonyl-benzyl)-carbamic acid | 6.2 | |
| | dydrogesterone-11-yl ester | | |
| 69 | Quinolin-6-yl-carbamic acid dydrogesterone-11-yl | 6.2 | |
| | ester | | |
| 71 | (3-Cyano-phenyl)-carbamic acid dydrogesterone- | 6.2 | |
| | 11-yl ester | | |
| 76 | (3-Bromo-4-methyl-phenyl)-carbamic acid | 6.2 | |
| | dydrogesterone-11-yl ester | | |
| 77 | (2-Methyl-4-oxo-4H-chromen-7-yl)-carbamic | 6.2 | |
| | acid dydrogesterone-11-yl ester | | |
| 78 | (3-Fluoro-benzyl)-carbamic acid dydrogesterone- | 6.4 | |
| | 11-yl ester | | |
| 79 | (2-Thiophen-2-yl-ethyl)-carbamic acid | 6.2 | |
| | dydrogesterone-11-yl ester | | |
| 81 | (3-Acetyl-phenyl)-carbamic acid dydrogesterone- | 6.6 | |
| | 11-yl ester | | |
| 82 | (4-Acetylamino-phenyl)-carbamic acid | 6.6 | |
| | dydrogesterone-11-yl ester | | |
| 85 | (3-Cyano-4-fluoro-phenyl)-carbamic acid | 6.3 | |
| | dydrogesterone-11-yl ester | | |
| 91 | (1H-Indol-5-yl)-carbamic acid dydrogesterone- | 6.2 | |
| | 11-yl ester | | |
| 92 | (3-Methylsulfanyl-phenyl)-carbamic acid | 6.2 | |
| | dydrogesterone-11-yl ester | | |
| 99 | (1-Methyl-1H-indazol-6-yl)-carbamic acid | 6.3 | |
| | dydrogesterone-11-yl ester | | |
| 101 | (4-Methyl-3-oxo-3,4-dihydro-2H- | 6.5 | |
| | benzo[1,4]oxazin-6-yl)-carbamic acid | | |
| | dydrogesterone-11-yl ester | | |
| 102 | (2-Methyl-3-oxo-3,4-dihydro-2H- | 6.3 | |
| | benzo[1,4]thiazin-6-yl)-carbamic acid | | |
| | dydrogesterone-11-yl ester | | |

### II. Progesterone-Dependent Alkaline Phosphatase Expression Assay

The progesterone-dependent modulation of alkaline phosphatase expression was examined using T47D human breast carcinoma cells [Keydar et al., 1979]. The assay was performed as previously described by Di Lorenzo et al. (1991) with the modification of using Dydrogesterone as comparative progestin to determine the antagonistic and agonistic activity.

### Procedure

The cell line was purchased from CLS Cell Lines Service (Hildastrasse 21, D-69214 Eppelheim, Germany). In brief, the cells were plated in 96-well plates at 40,000 cells/well using the following growth medium: RPMI 1640 with: 10% FBS, 1 mM Sodium Pyruvat MEM, 10 mM Hepes, 0.01 mg/ml Bovine insulin, and 25 μg/ml Gentamycin. After 24 h of cultivation, the growth medium was replaced with medium containing 2% fetal bovine serum and the test compounds were added to each well to achieve the appropriate compound concentration: For determination of agonistic activity only the test compounds were added; for measurement of antagonistic activity the test compounds and additionally Dydrogesterone as standard progesterone agonist was added to a final concentration of 1 nM. After 48 h of cultivation, the medium was removed and the cells were washed with 200 μl of Dulbecco's phosphate-buffered saline without calcium and magnesium (PBS(−)). Then the cells were fixed with 3.7% formaldehyde in phosphate-buffered saline for 15 min at 22° C. After washing the cells with PBS, 100 μl of a para-nitro-phenole (pNPP) solution (pNPP Liquid Substrate System; Sigma) was added to each well and incubated for 2 h at room temperature protected from light. The reaction was stopped with 100 μl 1N NaOH and the absorbance was measured with a spectrophotometer (Victor, Perkin Elmer) at 405 nm. The results are expressed as alkaline phosphatase induction (as 100% with 1 nM Dydrogesterone) or inhibition (against alkaline phosphatase induction by 1 nM Dydrogesterone) at a certain concentration of test compound.

### Calculations:

% stimulation=(effect compound−basal)/(effect dydro 1 nM−basal)*100% inhibition of 1 nM Dydro=100*{1-[(effect compound−basal)/(effect dydro 1 nM−basal)]}

For each compound the % inhibition (PI) and % stimulation (PS), respectively, at a compound concentration of 100 nM was determined. For selected compounds, the corresponding values were measured for several different concentrations, and subsequently were plotted against the concentration of the test compound, and used to calculate the IC50 value (for the antagonistic potency; the IC50 value is the concentration (nM), required to reduce the maximal response by 50%) and EC50 values (for the agonistic potency; the EC50 value is the effective concentration (nM) that produced 50% of the maximum response), respectively.

### Results

The results of the AP assay are presented in the following table 4.

TABLE 4

| | | AP Assay Results | | | |
|---|---|---|---|---|---|
| Compound | | PI | PS | | |
| No. | Name | [100 nM] | [100 nM] | pIC50 | pEC50 |
| 1 | Isopropyl-carbamic acid | 46.0 | −3.0 | 6.8 | |
| | dydrogesterone-11-yl ester | | | | |
| 11 | (4-Methoxy-phenyl)-carbamic acid | 39.0 | 0.0 | | |
| | dydrogesterone-11-yl ester | | | | |
| 12 | (3-Methoxy-phenyl)-carbamic acid 17- | 72.0 | 23.0 | 8.2 | |
| | ethoxy-dydrogesterone-11-yl ester | | | | |
| 13 | Benzo[1,3]dioxol-5-yl-carbamic acid | 73.0 | 5.0 | 7.5 | 6.4 |
| | dydrogesterone-11-yl ester | | | | |
| 14 | (3-Carbamoyl-phenyl)-carbamic acid | 38.0 | −1.0 | 7.3 | |
| | dydrogesterone-11-yl ester | | | | |
| 15 | (3-Methoxy-phenyl)-carbamic acid | 43.0 | −3.0 | 6.9 | |
| | dydrogesterone-11-yl ester | | | | |
| 22 | 4-Methoxy-benzoic acid | 55.0 | 29.0 | 7.7 | 7.9 |
| | dydrogesterone-11-yl ester | | | | |
| 23 | Benzoic acid 17-ethoxy- | 69.0 | 16.0 | 7.8 | |
| | dydrogesterone-11-yl ester | | | | |
| 24 | (3,4,5-Trimethoxy-phenyl)-carbamic | 88.0 | 0.0 | 7.3 | |
| | acid dydrogesterone-11-yl ester | | | | |
| 29 | (3-Methoxy-phenyl)-carbamic acid | 87.0 | 0.0 | 8.3 | |
| | (17α-ethoxy-1,2-methylene- | | | | |
| | dydrogesterone-11β-yl ester | | | | |
| 33 | [3-(N-Ethylcarbamoyl-oximino- | −29.0 | 56.0 | | 6.7 |
| | formyl)-phenyl]-carbamic acid | | | | |
| | dydrogesterone-11-yl ester | | | | |
| 34 | Benzo[1,3]dioxol-5-yl-carbamic acid | 5.0 | 95.0 | | |
| | 17-ethoxy-dydrogesterone-11-yl ester | | | | |
| 41 | (1H-Indazol-6-yl)-carbamic acid | 96.0 | 1.0 | 8.2 | |
| | dydrogesterone-11-yl ester | | | | |
| 42 | Benzo[1,3]dioxol-5-ylmethyl-carbamic | 6.0 | 76.0 | | 7.0 |
| | acid dydrogesterone-11-yl ester | | | | |
| 51 | Quinolin-3-yl-carbamic acid | 59.0 | 5.0 | 7.0 | |
| | dydrogesterone-11-yl ester | | | | |
| 55 | (4-Methoxy-benzyl)-carbamic acid | −79.0 | 143.0 | | 7.3 |
| | dydrogesterone-11-yl ester | | | | |
| 69 | Quinolin-6-yl-carbamic acid | 41.0 | 4.0 | 7.2 | |
| | dydrogesterone-11-yl ester | | | | |
| 70 | (2,3-Dihydro-benzo[1,4]dioxin-6-yl)- | 44.0 | 1.0 | 6.9 | |
| | carbamic acid dydrogesterone-11-yl | | | | |
| | ester | | | | |
| 74 | (3-Trifluoromethoxy-phenyl)- | −23.0 | 89.0 | | 6.3 |
| | carbamic acid dydrogesterone-11-yl | | | | |
| | ester | | | | |
| 75 | (4-Difluoromethoxy-phenyl)-carbamic | 59.0 | 0.0 | 6.9 | |
| | acid dydrogesterone-11-yl ester | | | | |
| 76 | (3-Bromo-4-methyl-phenyl)-carbamic | 67.0 | 4.0 | 7.3 | |
| | acid dydrogesterone-11-yl ester | | | | |
| 81 | (3-Acetyl-phenyl)-carbamic acid | 72.0 | 1.0 | 7.3 | |
| | dydrogesterone-11-yl ester | | | | |
| 82 | (4-Acetylamino-phenyl)-carbamic acid | 40.0 | 0.0 | | |
| | dydrogesterone-11-yl ester | | | | |
| 88 | (1H-Indol-6-yl)-carbamic acid | 48.0 | 0.0 | | |
| | dydrogesterone-11-yl ester | | | | |
| 91 | (1H-Indol-5-yl)-carbamic acid | 58.0 | 1.0 | 7.0 | |
| | dydrogesterone-11-yl ester | | | | |
| 92 | (3-Methylsulfanyl-phenyl)-carbamic | 55.0 | 1.0 | 7.0 | |
| | acid dydrogesterone-11-yl ester | | | | |
| 94 | (4-Methylsulfanyl-phenyl)-carbamic | 61.0 | 1.0 | 7.0 | |
| | acid dydrogesterone-11-yl ester | | | | |
| 95 | (3-Acetylamino-phenyl)-carbamic acid | 50.0 | 1.0 | 6.8 | |
| | dydrogesterone-11-yl ester | | | | |
| 98 | (1,1-Dioxo-1H-1lambda*6*- | 61.0 | 1.0 | 7.0 | |
| | benzo[b]thiophen-6-yl)-carbamic acid | | | | |
| | dydrogesterone-11-yl ester | | | | |
| 99 | (1-Methyl-1H-indazol-6-yl)-carbamic | 70.0 | 0.0 | 7.1 | |
| | acid dydrogesterone-11-yl ester | | | | |
| 100 | [3-(2-Oxo-pyrrolidin-1-yl)-phenyl]- | 45.0 | 1.0 | 6.6 | |
| | carbamic acid dydrogesterone-11-yl | | | | |
| | ester | | | | |
| 101 | (4-Methyl-3-oxo-3,4-dihydro-2H- | 72.0 | 0.0 | 7.1 | |
| | benzo[1,4]oxazin-6-yl)-carbamic acid | | | | |
| | dydrogesterone-11-yl ester | | | | |
| 102 | (2-Methyl-3-oxo-3,4-dihydro-2H- | 98.0 | 0.0 | 8.1 | |
| | benzo[1,4]thiazin-6-yl)-carbamic acid | | | | |
| | dydrogesterone-11-yl ester | | | | |
| 103 | (3-Pyrrol-1-yl-phenyl)-carbamic acid | 52.0 | 21.0 | 7.2 | |
| | dydrogesterone-11-yl ester | | | | |
| 104 | (3-Methoxy-5-tetrazol-1-yl-phenyl)- | 45.0 | 1.0 | 6.9 | |
| | carbamic acid dydrogesterone-11-yl | | | | |
| | ester | | | | |

### III. Clauberg-McPhail Assay

The in vivo activity of selected PR modulator compounds of the present invention is evaluated utilizing the McPhail assay. The Clauberg or McPhail assay is a classic assay utilizing rabbits to measure progestational activity and allows the assessment of the progestagenic and antiprogestagenic effects of the compounds [McPhail, 1934^{l}]. The reason rabbit is used is because the results observed in rabbit have proved to be a good indicator and predictor of activity in the human. In this assay, immature rabbits are treated initially with estradiol, which induces growth in the uterus. This is followed by treatment with a progestin, which causes a large change in the glandular content of the uterus. It is this change in the glandular component, which is a measure of the progestational activity of a progestin. The measurement of these glandular changes is carried out histologically using stained sections of the uterus.

### Procedure

The test is performed in 6-week-old juvenile female rabbits (New Zealand white). From days 1 to 6, all rabbits are primed with 5.0 μg/kg/day 17β-estradiol (s.c., 0.5 ml/kg/day) in order to induce proliferation of the endometrium. From days 7 to 11, the test compound is applied (0.5 ml/kg/day) at doses in the range of 0.001 to 10 mg/kg/day. A group which receives only vehicle after estradiol priming serves as a negative control. A second group which receives only progesterone in order to induce endometrial differentiation after estradiol priming is used as a positive control. The antagonistc activity is measured by the combined administration of progesterone and the test compound in the appropriate dosages.

### Evaluation

Autopsy is performed on day 12. As a parameter for progestagenic activity, the McPhail score (i.e., the degree of differentiation) is determined by means of light microscopy (scores: 1 to 4; 1=no glandular differentiation, 4=maximal differentiation).

By definition, progesterone produces a maximum McPhail score of 4; treatment with a PR antagonist in the absence of progesterone leads to a McPhail score which is distinctly lower in score than 4 at the plateau of the dose response curve at the clinically relevant doses (i.e. 0.01 mg-10 mg/rabbit). Preferably, a SPRM leads to a McPhail score which is higher than that under any dose of the PR antagonist RU 486 (Mifepristone). The capacity of SPRMs to antagonize progesterone function can also be tested in the McPhail test using a progesterone dose which induces a McPhail score ranging between 3 and 4. A SPRM inhibits the effect of progesterone to a significant degree, but the maximum inhibition is below that which is inducible with RU 486 or other pure antiprogestins, such as onapristone.

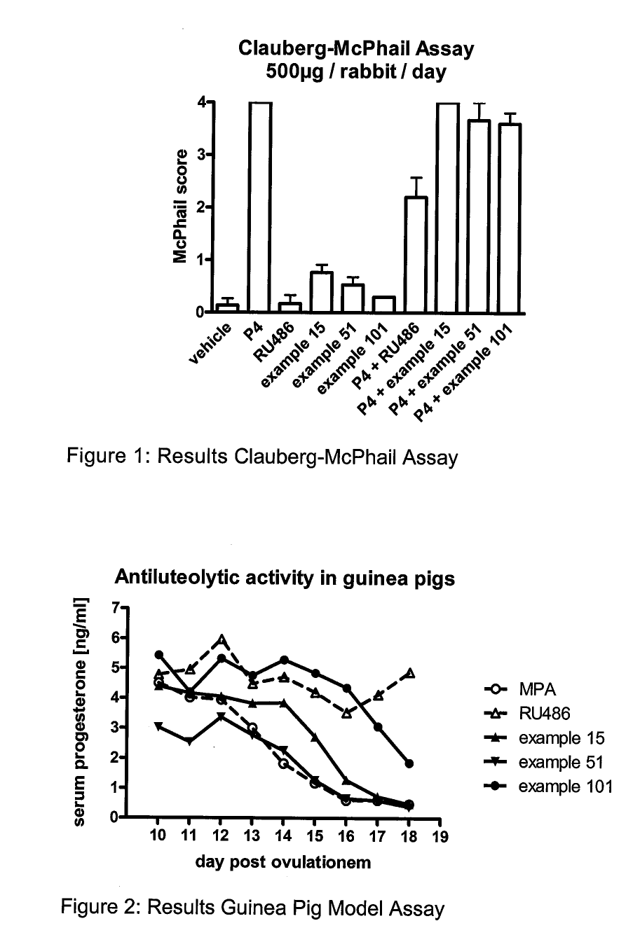

### Results (FIG. 1)

Preferred compounds of the invention acting as SPRMs display a McPhail score which is above that of RU486, when administered alone (agonistic mode). In the antagonistic mode of the assay, the preferred SPRM compounds of the invention show inhibition of the effect of the administered progesterone; however, this inhibitiory activity is clearly below that which is inducible with the strong antiprogestin RU486.

### IV. Guinea Pig Model

An assay for assessing the progesterone antagonists (PAs), Progesterone agonists (Ps) and progesterone receptor modulators (PRMs) with respect to PR agonistic and antagonistic activities in vivo is described by Elger et al [2000]^{lv} and within WO 04/014935^{xix} using cycling guinea pigs. In this assay, pure PR antagonists inhibit luteolysis at the end of the ovarian cycle, whereas PR agonist and SPRMs support luteolysis, i.e. this is a very sensitive in vivo method to reveal residual agonistic activity of SPRMs. Inhibition of luteolysis is reflected by elevated serum progesterone levels at day 10-17 and inhibition of uterine prostaglandin F2_{α}, as well as by certain histological characteristics in uterus and ovary, such as increased expression of progesterone receptors and decreased glandular differentiation in the uterus, as well as persistence of large intact corpora lutea up to day 18.

### Procedure

Adult female guinea pigs (strain Dunkin Hartley, Crl:HA; body weight 500-700 g) are purchased from Charles River (Sulzfeld, Germany). Blood samples are drawn from the Vena saphena three times a week to monitor ovarian cycles by determination of progesterone levels. Animals showing at least two regular ovarian cycles are treated once daily with 10 mg/kg s.c. of test compounds dissolved in benzyl benzoate/castor oil (1+4 vol), on days 10-17 of the cycle. During this treatment period blood samples for progesterone determination are collected once daily. On day 18 the animals are killed by CO₂-asphyxiation. Ovaries and uteri are collected and processed for histological analysis.

### Evaluation

Antiluteolytic activity is evaluated by assessment of serum progesterone profiles throughout the treatment period from day 10 to day 17. When antiprogestins like mifepristone (RU486) are administered, then Progesterone levels do not decline, i.e. luteolysis is inhibited. With progestins (e.g. dydrogesterone) and SPRMs, progesterone levels decrease meaning that no inhibition of luteolysis is observed.

### Results (FIG. 2)

Antiluteolytic activity of the compounds of the invention is not as strong as that of the PR antagonist RU486, but stronger than pure PR agonists, such as medroxyprogesterone acetate (MPA).

### V. Summary

The compounds and pharmaceutical compositions of the present invention may be extremely potent modulators of the PR, while however their absolute agonistic activity remains below that of natural progesterone in the plateau of the dose response curve and their absolute antagonistc activity remains below that of known antiprogestins such as onapristone or mifepristone (RU 486). Additionally, some compounds of the invention might even show only pure antagonistic or only pure agonist activity on the PR.

For example, the compounds and compositions of the present invention may display 50% maximal activation of the progesterone receptor at a concentration of less than 10 μM. Some compounds and compositions of the present invention may display 50% maximal activation of PR at a concentration of less than 1 μM, and some may display such activity at a concentration of less than 100 nM or even 10 nM.

Furthermore, some compounds and compositions of the present invention may display 50% maximal inhibition of the progesterone receptor at a concentration of less than 10 μM. Some compounds and compositions of the present invention may display 50% maximal inhibition of PR at a concentration of less than 1 μM, and some may display such activity at a concentration of less than 100 nM or even 10 nM.

In a further embodiment of the present invention, the compounds provide for 50% maximum inhibition measured in the antagonistic mode of the AP assay at a concentration of less than 1 μM, preferably less than 100 nM and even more preferred less than 10 nM, and additionally for 50% maximum activation measured using the agonistic AP assay as described here within at a concentration of less than 10 μM, preferably less than 1 μM and even more preferred less than 100 nM.

### Example Pharmaceutical Compositions

Set forth below, by way of example and not of limitation, are several pharmaceutical compositions utilizing the some preferred active compounds for systemic use or topical application. Other compounds of the invention or combinations thereof, may be used in place of (or in addition to) said compounds. The concentration of the active ingredient may be varied over a wide range as discussed herein. The amounts and types of ingredients that may be included are well known in the art.

### I. Hard Gelatin Capsules

Hard gelatin capsules can be prepared using the following ingredients:

| Ingredient | Quantity (mg/capsule) |
|---|---|
| COMPOUND No. 13 | 10 |
| Starch, dried | 95 |
| Magnesium stearate | 14 |
| Total | 120 |

The above ingredients are mixed and filled into hard gelatin capsules in 120 mg quantities.

### II. Tablets

A tablet is prepared using the ingredients below:

| Ingredient | Quantity (mg/tablet) |
|---|---|
| COMPOUND No. 29 | 10 |
| Cellulose, microcrystalline | 200 |
| Silicon dioxide, fumed | 10 |
| Stearic acid | 10 |
| Total | 230 |

The components are blended and compressed to form tablets each weighing 230 mg.

### III. Suppositories

Suppositories, each containing 1 mg of active ingredient, may be made as follows:

| Ingredient | Quantity (mg/suppository) |
|---|---|
| COMPOUND No. 22 | 20 |
| Saturated fatty acid glycerides | 2,000 |
| Total | 2,020 |

The active ingredient is passed through a appropriately sized mesh sieve and suspended in the saturated fatty acid glycerides previously melted using the minimum heat necessary. The mixture is then poured into a suppository mold of normal 2 g capacity and allowed to cool.

### IV. Intravenous Formulation

An intravenous formulation may be prepared as follows:

| Ingredient | Quantity | |
|---|---|---|
| COMPOUND No. 13 | 1 | g |
| Arlatone G ™ | 100 | ml |
| EtOH | 100 | ml |
| Water, sterile | 800 | ml |

The compound is dissolved in the Arlatone G™, EtOH and water, and then the solution is slowly diluted with further water.

### General Provisions

The scope of the invention is not to be limited by the description of the examples. Modifications and alterations of the present invention will be apparent to those skilled in the art without departing from the scope and spirit of the present invention. Therefore, it will be appreciated that the scope of this invention is to be defined by the appended claims, rather than by the specific examples which have been presented by way of example.

### CITED LITERATURE

Citation of any reference throughout this application is not to be construed as an admission that such reference is prior art to the present application.
ⁱ McPhail M K (1934) “The assay of progestin” J Physiol 83:145-156
ⁱⁱ Di Lorenzy D.; Albertine A.; Zava D. (1991): Progestin regulation of alkaline phosphatase in the human breast cancer cell line T47D. Cancer Research 51: 4470-4475
ⁱⁱⁱ Sobek L, Di Lorenzo D, Oettel M, Kaufmann G. (1994) “Normal and stable transfected cancer cell lines: tools for a screening of progestogenic, antiprogestogenic and antiglucocorticoid substances.” Methods Find Exp Clin Pharmacol. 16(7):545-51. Review
^{iv} WO 01/15679
^{v} Spitz IM. “Progesterone antagonists and progesterone receptor modulators: an overview” Steroids. 2003 68(10-13):981-93
^{vi} Tamaya T, Fujimoto J, Okada H. “Comparison of cellular levels of steroid receptors in uterine leiomyoma and myometrium.” Acta Obstet Gynecol Scand. 1985; 64(4):307-9
^{vii} Zahradnik (1992) “Menstruation”. In Kaser O et al. (editors) Gynaekologie und Geburtshilfe [Gynecology and Obstetrics], Vol. 1/2, Georg Thieme Verlag Stuttgart, New York: 7.31-7.51
^{viii} Mencaglia, L., Perino, A., Hamon, J. (1987) Hysteroscopy in Perimenopausal and Postmenopausal Women with Abnormal Uterine Bleeding. J. Reprod. Med. 32:577
^{ix} Spitz IM. “Progesterone receptor antagonists and selective progesterone receptor modulators (SPRMs)” Semin Reprod Med. 2005 23(1):3-7
^{x} Spitz IM. “Progesterone antagonists and progesterone receptor modulators” Expert Opin Investig Drugs. 2003 12(10):1693-707. Review
^{xi} Zhang P, Fensome A, Wrobel J, Winneker R, Zhang Z (2003) “Non-steroidal progesterone receptor modulators” Expert Opin Ther Patents 13(12):1839-1847
^{xii} Schubert G, Elger W, Kaufmann G, Schneider B, Reddersen G, Chwalisz K. (2005) “Discovery, chemistry, and reproductive pharmacology of asoprisnil and related 11beta-benzaldoxime substituted selective progesterone receptor modulators (SPRMs).” Semin Reprod Med. 23(1):58-73. Review
^{xiii} EP 1229906 (WO 01/15679)
^{xiv} EP 0648778 (U.S. Pat. No. 5,693,628)
^{xv} WO 99/45023
^{xvi} EP 909764
^{xvii} WO 01/44267
^{xviii} WO 03/093292
^{xix} WO 04/014935
^{xx} WO 01/18025
^{xxi} WO 00/34306
^{xxii} WO 99/62928
^{xxiii} WO 99/62929
^{xxiv} WO 99/45022
^{xxv} EP 0152138B1 (U.S. Pat. No. 4,601,855)
^{xxvi} EP 0558119B1 (U.S. Pat. No. 5,304,291)
^{xxvii} U.S. Pat. No. 3,937,700
^{xxviii} BE 652,597
^{xxix} U.S. Pat. No. 3,304,314
^{xxx} U.S. Pat. No. 3,555,053
^{xxxi} Westerhof P., Hartog J. & Halkes S J (1965) “Investigations on Sterols XXVI: Synthesis and properties of 6-substituted 9β,10α-steroids” Recueil des Travaux Chimiques des Pays-Bas, 84:863-884
^{xxxii} Hartog J, Wittelaar J I, Morsink L, de Wachter A M (1972) “Investigations on sterols. 39. Synthesis and progestational activities of some 16-methylene-17α-acetoxy-9β,10α-pregna-4,6-diene-3,20-dione derivatives” J Med. Chem. 1972 December; 15(12):1292-7
^{xxxiii} Halkes S J, Hartog J, Morsink L, de Wachter A M. (1972) “Investigations on sterols. 38. Synthesis of 1,2-methylene-17α-acetoxy-9β,10α-pregnanes, a class of potent progestational agents” J Med Chem, 15(12):1288-92
^{xxxiv} Van Moorselaar R. & Halkes S J (1969) “Investigations on Sterols XXXIII: Synthesis of 18-alkyl-9β,10α-pregnane derivatives” Recueil des Travaux Chimiques des Pays-Bas, 88(7):737-51
^{xxxv} GB 1,111,320
^{xxxvi} Stella J (2004) “Pro-drugs as therapeutics” Expert Opin. Ther. Patents 14(3): 277-280.
^{xxxvii} Ettmayer P et al. (2004) “Lessons learned from marketed and investigational pro-drugs” J. Med. Chem. 47: 2393-2404.
^{xxxviii} WO 02/054064
^{xxxix} EP 0977555 A1
^{xl} U.S. Pat. No. 5,993,856
^{xli} U.S. Pat. No. 6,652,874
x^{lii} U.S. Pat. No. 6,416,778
^{xliii} Halkes S J & Van Moorselaar R (1969) “Investigations on Sterols XXXIV: Synthesis of 18-methyl-9β,10α-androstanes” Recueil des Travaux Chimiques des Pays-Bas, 88(7):752-765
^{xliv} BE 577,615
^{xlv} EP 0028309
^{xlvi} U.S. Pat. No. 6,046,023
^{xlvii} van der Sijde D, de Flines J, van derWaard W F (1966) “Microbial transformation of 9β,10α-Steroids: III. 11-Hydroxylation and side chain degradation of 9β,10α-Steroids” Recueil des Travaux Chimiques des Pays-Bas, 85:721-730
^{xlviii} Saucy G, Els H, Mikesch F, Fürst A (1966) “Über 9β,10α-Steroide: Strukturaufklärung von mikrobiologischen Umsetzungsprodukten mit Sauerstoff-Funktion in Stellung 11” Helv Chim Acta 49(5):1529-1542
^{xlix} WO2007082891
^{l} Cairns T L and McKusick B C (1959) “Reactions of 2-thienyl chloride and 5-chloro-2-thienyl chloride” J Org Chem 15: 790-794
^{li} Anderson G W & McGregor A C (1957) “t-Butyloxycarbonylamino Acids and Their Use in Peptide Synthesis” J American Chem Soc 79(23): 6180-6183.
^{lii} Boden P R, Higginbottom M, Hill D R, Horwell D C, Hughes J, Rees D C, Roberts E, Singh L, Suman-Chauhan N, and Woodruff G N (1993) “Cholecystokinin dipeptoid antagonists: design, synthesis, and anxiolytic profile of some novel CCK-A and CCK-B selective and mixed CCK-A/CCK-B antagonists” J Med Chem 36(5): 552-565.
^{liii} Rosling A; Fueloep F; Sillanpaeae R; Mattinen J (1997) “2-[Arylamino(imino)]perhydropyrido-[1,2-d][1,3,4]oxadiazine and 2-[Aryl-amino(imino)]-perhydropyrrolo[1,2-d]-[1,3,4]oxadiazine: Heterocyclic Ring Systems Involving a Bridge-Head Nitrogen” Heterocycles 45(5): 927-942.
^{liv} Graf R (1963) “Umsetzungen mit N-Carbonyl-sulfamidsäure-chlorid, II. Alkohole und Phenole” Chem Ber 96(1): 56-57.

What is claimed is:

1. A compound corresponding to formula (1) 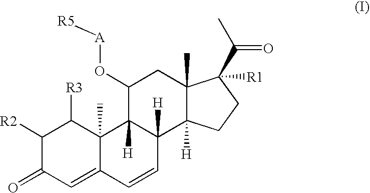
Wherein
A represents —CO—, —CO-NR6-, —CO-NR4-NR6- or —CO—NH—SO₂-NR6-;
R1 is selected from hydrogen, —OH, —O—(C₁-C₄)alkyl and —O—CO—(C₁-C₄)alkyl;
R2 and R3 are both hydrogen or together form a methylene group;
R4 is selected from hydrogen and —(C₁-C₄)alkyl;
R5 is selected from
(a) aryl and aryl-(C₁-C₄)alkyl, wherein
(iii) the aryl group is unsubstituted; or
(iv) the aryl moiety of the aryl-(C₁-C₄)alkyl group is unsubstituted, or
(v) the aryl moiety of the aryl or aryl-(C₁-C₄)alkyl group is substituted with one or more substituents independently selected from —O—R⁹, —S—R⁹, —O—CO—NHR¹⁰, —O—CO—R¹¹, —CO—R¹¹, —SO₂—R¹¹, —CO—O—R⁹, —CO—NR⁷R⁸, —SO₂—NR⁷R⁸, —CN, —CH═N—O—R¹², —CH═N—O—CO—NHR¹⁰, —CH═N—O—CO—R¹¹, —CH═N—O—CO—O—R⁹, —(C₁-C₄)alkyl-CO—O—R⁹, —NR⁷R⁸, —NR¹³—CO—R¹¹, —NR¹³—CO—NHR¹⁰, —NR¹³—CO—O—R⁹, —NR¹³—SO₂—R¹¹, halogen, —(C₁-C₄)alkyl, halogenated —(C₁-C₄)alkyl, cycloheteroalkyl, aryl and heteroaryl, the number of said substituents being 1, 2, 3, 4 or 5 for halogen, and 1, 2 or 3 for any combination of said substituents, and wherein the cycloheteroalkyl or heteroaryl is optionally substituted with one or two substituents independently selected from oxo, halogen, —(C₁-C₄)alkyl and halogenated —(C₁-C₄)alkyl; or
(vi) the aryl moiety of the aryl or aryl-(C₁-C₄)alkyl group is substituted by two groups which are attached to adjacent carbon atoms and are combined into a saturated or partly unsaturated cyclic 5-, 6-, 7- or 8-membered ring system, optionally containing 1, 2 or 3 heteroatoms selected from N, O and S, the number of N atoms being 0, 1, 2 or 3 and the number of O and S atoms each being 0, 1 or 2; whereby the cyclic ring system is optionally substituted by one or two substituents independently selected from oxo, —(C₁-C₄)alkyl and halogenated —(C₁-C₄)alkyl;
(b) heteroaryl and heteroaryl-(C₁-C₄)alkyl,
wherein the heteroaryl moiety of the heteroaryl or heteroaryl-(C₁-C₄)alkyl group is optionally substituted with one or more substituents independently selected from —O—R⁹, —S—R⁹, —O—CO—NHR¹⁰, —O—CO—R¹¹, —CO—R¹¹, —SO₂—R¹¹, —CO—O—R⁹, —CO—NR⁷R⁸, —SO₂—NR⁷R⁸, —CN, —CH═N—O—R¹², —CH═N—O—CO—NHR¹⁰, —CH═N—O—CO—R¹¹, —CH═N—O—CO—O—R⁹, —(C₁-C₄)alkyl-CO—O—R⁹, —NR⁷R⁸, —NR¹³—CO—R¹¹, —NR¹³—CO—NHR¹⁰, —NR¹³—CO—O—R⁹, —NR¹³—SO₂—R¹¹, halogen, —(C₁-C₄)alkyl, halogenated —(C₁-C₄)alkyl and aryl, the number of said substituents being 1, 2, 3, 4 or 5 for halogen, and 1, 2 or 3 for any combination of said substituents;
(c) cycloheteroalkyl and cycloheteroalkyl-(C₁-C₄)alkyl,
wherein the cycloheteroalkyl moiety of the cycloheteroalkyl or cycloheteroalkyl-(C₁-C₄)alkyl group is optionally substituted with 1, 2, 3 or 4 substituents independently selected from the group consisting of oxo, —(C₁-C₄)alkyl and aryl; and
(d) (C₁-C₈)alkyl, wherein the (C₁-C₈)alkyl group is
(vii) unsubstituted under the proviso that A represents —CO-NR6-, —CO-NR4-NR6- or —CO—NH—SO₂-NR6-, or
(viii) substituted with one or two substituents independently selected from the group consisting of halogen, —O—R⁹, —S—R⁹, —NR⁷R⁸, —CO—OR⁹, —CO—R¹¹, —CO—NR⁷R⁸ and —CN;
wherein the —(C₁-C₄)alkyl moiety of the aryl-(C₁-C₄)alkyl, heteroaryl-(C₁-C₄)alkyl or cycloheteroalkyl-(C₁-C₄)alkyl group in R5 is optionally substituted with one or two substituents independently selected from oxo and hydroxyl;
R6 is selected from hydrogen and —(C₁-C₈)alkyl, optionally substituted with —O—R⁹ or halogen, the number of said substituents being 1, 2 or 3 for halogen, and 1 or 2 for any combination of said halogen or —O—R⁹ moieties; or
R5 and R6 together with the nitrogen atom to which R5 and R6 are attached form a heterocyclic 5-, 6-, 7- or 8-membered ring system, which is saturated, partly unsaturated, or aromatic; which optionally contains 1, 2 or 3 additional heteroatoms selected from N, O and S, the number of additional N atoms being 0, 1, 2 or 3 and the number of O and S atoms each being 0, 1 or 2; which ring is optionally part of a multiple condensed ring-system; and which ring system is optionally substituted with an aryl group, the aryl group being optionally substituted with one or two substituents independently selected from —O—R¹⁴, halogen, —(C₁-C₄)alkyl and halogenated —(C₁-C₄)alkyl;
wherein R⁷, R⁸, R⁹, R¹⁰, R¹¹, R¹², R¹³ and R¹⁴ are independently selected from the group consisting of hydrogen, —(C₁-C₄)alkyl, halogenated —(C₁-C₄)alkyl, aryl and aryl-(C₁-C₄)alkyl;
or R⁷ and R⁸ form together with the nitrogen atom, where R⁷ and R⁸ are attached, a heterocyclic 5-, 6-, 7- or 8-membered ring system, which is saturated, partly unsaturated, or aromatic; and which optionally contains 1 or 2 additional heteroatoms selected from N, O and S, the number of additional N atoms being 0, 1 or 2 and the number of O and S atoms each being 0 or 1;
or a tautomer or salt thereof.

2. A compound as claimed in claim 1, wherein A represents —CO— or —CO-NR6-.

3. A compound as claimed in claim 2, wherein A represents —CO-NR6-.

4. A compound as claimed in claim 1, wherein R1 is hydrogen or —O—(C₁-C₄)alkyl.

5. A compound as claimed in claim 1, wherein
A represents —CO—; and
R1 is —O—(C₁-C₄)alkyl or —O—CO—(C₁-C₄)alkyl.

6. A compound as claimed in claim 1, wherein R1 is —O—(C₁-C₄)alkyl.

7. A compound as claimed in claim 1, wherein
A represents —CO—;
R1 is selected from the group consisting of hydrogen, —OH, —O—(C₁-C₄)alkyl and —O—CO—(C₁-C₄)alkyl; and
R2 and R3 together form a methylene group.

8. A compound as claimed in claim 7, wherein R1 is hydrogen or —O—(C₁-C₄)alkyl.

9. A compound as claimed in claim 1, wherein said compound is an optically pure enantiomer corresponding to formula (Ib)

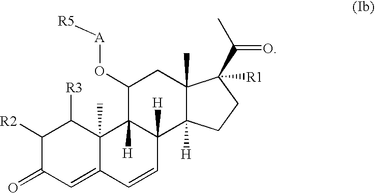

10. A compound as claimed in claim 1, wherein R5 is selected from
(a) aryl and aryl-(C₁-C₄)alkyl, wherein
(i) the aryl group is unsubstituted; or
(ii) the aryl moiety of the aryl-(C₁-C₄)alkyl group is unsubstituted, or
(iii) the aryl moiety of the aryl or aryl-(C₁-C₄)alkyl group is substituted with one or more substituents independently selected from —O—R⁹, —S—R⁹, —O—CO—NHR¹⁰, —CO—R¹¹, —SO₂—R¹¹, —CO—NR⁷R⁸, —SO₂—NR⁷R⁸, —CN, —CH═N—O—R¹¹, —CH═N—O—CO—NHR¹⁰, —NR⁷R⁸, —NR¹³—CO—R¹¹, —NR¹³—SO₂—R¹¹, halogen, —(C₁-C₄)alkyl, halogenated —(C₁-C₄)alkyl, cycloheteroalkyl and heteroaryl, the number of said substituents being 1, 2, 3, 4 or 5 for halogen, and 1, 2 or 3 for any combination of said substituents, and wherein the cycloheteroalkyl or heteroaryl is optionally substituted with oxo or —(C₁-C₄)alkyl; or
(iv) the aryl moiety of the aryl or aryl-(C₁-C₄)alkyl group is substituted by two groups which are attached to adjacent carbon atoms and are combined into a saturated or partly unsaturated cyclic 5- or 6-membered ring system, optionally containing 1, 2 or 3 heteroatoms selected from N, O and S, the number of N atoms being 0, 1, 2 or 3 and the number of O and S atoms each being 0, 1 or 2; whereby the cyclic ring system is optionally substituted by one or two substituents independently selected from oxo and —(C₁-C₄)alkyl;
(b) heteroaryl and heteroaryl-(C₁-C₄)alkyl,
wherein the heteroaryl moiety of the heteroaryl or heteroaryl-(C₁-C₄)alkyl group is optionally substituted with a —(C₁-C₄)alkyl-CO—O—R⁹, —(C₁-C₄)alkyl or aryl group;
(c) cycloheteroalkyl and cycloheteroalkyl-(C₁-C₄)alkyl,
wherein the cycloheteroalkyl moiety of the cycloheteroalkyl or cycloheteroalkyl-(C₁-C₄)alkyl group is optionally substituted with 1, 2, 3 or 4 substituents independently selected from the group consisting of oxo, —(C₁-C₄)alkyl and aryl; and
(d) —(C₁-C₆)alkyl, wherein the —(C₁-C₆)alkyl group is
(i) unsubstituted under the proviso that A represents —CO-NR6-, —CO-NR4-NR6- or —CO—NH—SO₂-NR6-, or
(ii) substituted with one or two substituents independently selected from the group consisting of —O—R⁹ and —CN;
wherein the —(C₁-C₄)alkyl moiety of the aryl-(C₁-C₄)alkyl, heteroaryl-(C₁-C₄)alkyl or cycloheteroalkyl-(C₁-C₄)alkyl group in R5 is optionally substituted with an oxo group; and
R6 is hydrogen or —(C₁-C₄)alkyl, optionally substituted with an —O—R⁹ group; or
R5 and R6 together with the nitrogen atom to which R5 and R6 are attached form a heterocyclic 5- or 6-membered saturated ring system; which optionally contains 1 or 2 additional heteroatoms selected from N, O and S, the number of additional N atoms being 0, 1 or 2, and the number of O and S atoms each being 0 or 1; and which ring system is optionally substituted with an aryl group optionally substituted with —O—R¹⁴ or halogenated —(C₁-C₄)alkyl;
wherein R⁷, R⁸, R⁹, R¹⁰, R¹¹, R¹², R¹³ and R¹⁴ are each independently selected from the group consisting of hydrogen, —(C₁-C₄)alkyl, and halogenated —(C₁-C₄)alkyl.

11. A compound as claimed in claim 1, wherein R5 only represents an unsubstituted aryl group when R1 is —O—(C₁-C₄)alkyl or —O—CO—(C₁-C₄)alkyl.

12. A compound as claimed in claim 1, wherein R5 is selected from
(a) phenyl and phenyl-(C₁-C₂)alkyl, wherein
(i) the phenyl group is unsubstituted; or
(ii) the phenyl moiety of the phenyl-(C₁-C₂)alkyl group is unsubstituted, or
(iii) the phenyl moiety of the phenyl or phenyl-(C₁-C₂)alkyl group is substituted with one or more substituents independently selected from —O—R⁹, —S—R⁹, —CO—R¹¹, —CO—O—R⁹, —CO—NR⁷R⁸, —CN, —NH—CO—R¹¹, halogen, halogenated —(C₁-C₄)alkyl, —(C₁-C₄)alkyl, pyrrolidinyl, morpholinyl, pyrrolyl and tetrazolyl, the number of said substituents being 1, 2 or 3 for any combination of said substituents, and wherein the pyrrolidinyl is optionally substituted with an oxo group; or
(iv) the phenyl moiety of the phenyl or phenyl-(C₁-C₂)alkyl group is substituted by two groups which are attached to adjacent carbon atoms and are combined into a saturated or partly unsaturated cyclic 5- or 6-membered ring system, optionally containing 1 or 2 heteroatoms selected from N, O and S, the number of N atoms being 0, 1 or 2 and the number of O and S atoms each being 0, 1 or 2; whereby the cyclic ring system is optionally substituted by one or two substituents independently selected from oxo and —(C₁-C₄)alkyl;
(b) heteroaryl,
wherein the heteroaryl moiety of the heteroaryl or heteroaryl-(C₁-C₄)alkyl group is selected from 1H-indazolyl, quinolinyl, 1H-indolyl and 1,1-dioxo-1H-benzo[b]thienyl, and optionally substituted with —(C₁-C₄)alkyl; and
(c) —(C₁-C₄)alkyl, under the proviso that A represents —CO-NR6-, —CO-NR4-NR6- or —CO—NH—SO₂-NR6-; and
R6 is hydrogen or —(C₁-C₄)alkyl; or
R5 and R6 together with the nitrogen atom to which R5 and R6 are attached form a heterocyclic 6-membered saturated ring system; which optionally contains 1 additional N or O atom; and which ring system is optionally substituted with a phenyl group optionally substituted with —O—R¹⁴, —(C₁-C₄)alkyl or halogenated —(C₁-C₄)alkyl;
wherein R⁷, R⁸, R⁹, R¹¹ and R¹⁴ are each independently selected from the group consisting of hydrogen, —(C₁-C₄)alkyl and halogenated —(C₁-C₄)alkyl.

13. A compound as claimed in claim 12, wherein R5 only represents an unsubstituted phenyl group when R1 is —O—(C₁-C₄)alkyl or —O—CO—(C₁-C₄)alkyl.

14. A compound as claimed in claim 1, selected from the group consisting of:
(1,1-Dioxo-1H-1lambda*6*-benzo[b]thiophen-6-yl)-carbamic acid dydrogesterone-11β-yl ester;
(1H-Indazol-6-yl)-carbamic acid dydrogesterone-11β-yl ester;
(1H-Indol-5-yl)-carbamic acid dydrogesterone-11β-yl ester;
(1-Methyl-1H-indazol-6-yl)-carbamic acid dydrogesterone-11β-yl ester;
(2,3-Dihydro-benzo[1,4]dioxin-6-yl)-carbamic acid dydrogesterone-11β-yl ester;
(2-Methoxy-phenyl)-carbamic acid dydrogesterone-11β-yl ester;
(2-Methyl-3-oxo-3,4-dihydro-2H-benzo[1,4]thiazin-6-yl)-carbamic acid dydrogesterone-11β-yl ester;
(3,4,5-Trimethoxy-phenyl)-carbamic acid dydrogesterone-11β-yl ester;
(3-Acetylamino-phenyl)-carbamic acid dydrogesterone-11β-yl ester;
(3-Acetyl-phenyl)-carbamic acid dydrogesterone-11β-yl ester;
(3-Bromo-4-methyl-phenyl)-carbamic acid dydrogesterone-11β-yl ester;
(3-Carbamoyl-phenyl)-carbamic acid dydrogesterone-11β-yl ester;
(3-Cyano-4-fluoro-phenyl)-carbamic acid dydrogesterone-11β-yl ester;
(3-Cyano-phenyl)-carbamic acid dydrogesterone-11β-yl ester;
(3-Methoxy-5-tetrazol-1-yl-phenyl)-carbamic acid dydrogesterone-11β-yl ester;
(3-Methoxy-phenyl)-carbamic acid 17α-ethoxy-1,2-methylen-dydrogesterone-11β-yl ester;
(3-Methoxy-phenyl)-carbamic acid 17α-ethoxy-dydrogesterone-11β-yl ester;
(3-Methoxy-phenyl)-carbamic acid dydrogesterone-11β-yl ester;
(3-Methylsulfanyl-phenyl)-carbamic acid dydrogesterone-11β-yl ester;
(3-Pyrrol-1-yl-phenyl)-carbamic acid dydrogesterone-11β-yl ester;
(3-Trifluoromethoxy-phenyl)-carbamic acid dydrogesterone-11β-yl ester;
(4-Acetylamino-phenyl)-carbamic acid dydrogesterone-11β-yl ester;
(4-Difluoromethoxy-phenyl)-carbamic acid dydrogesterone-11β-yl ester;
(4-Methoxy-benzyl)-carbamic acid dydrogesterone-11β-yl ester;
(4-Methoxy-phenyl)-carbamic acid dydrogesterone-11β-yl ester;
(4-Methyl-3-oxo-3,4-dihydro-2H-benzo[1,4]oxazin-6-yl)-carbamic acid dydrogesterone-11β-yl ester;
(4-Methylsulfanyl-phenyl)-carbamic acid dydrogesterone-11β-yl ester;
(4-Morpholin-4-yl-phenyl)-carbamic acid dydrogesterone-11β-yl ester;
(4-Trifluoromethoxy-phenyl)-carbamic acid dydrogesterone-11β-yl ester;
[3-(2-Oxo-pyrrolidin-1-yl)-phenyl]-carbamic acid dydrogesterone-11β-yl ester;
4-(2-Methoxy-phenyl)-piperazine-1-carboxylic acid dydrogesterone-11β-yl ester;
4-(3-Trifluoromethyl-phenyl)-piperazine-1-carboxylic acid dydrogesterone-11β-yl ester;
4-Methoxy-benzoic acid dydrogesterone-11β-yl ester;
Benzo[1,3]dioxol-5-yl-carbamic acid 17-ethoxy-dydrogesterone-11β-yl ester;
Benzo[1,3]dioxol-5-yl-carbamic acid dydrogesterone-11β-yl ester;
Benzo[1,3]dioxol-5-ylmethyl-carbamic acid dydrogesterone-11β-yl ester;
Benzoic acid 17α-ethoxy-dydrogesterone-11β-yl ester;
Benzoic acid dydrogesterone-11β-yl ester;
Isopropyl-carbamic acid dydrogesterone-11β-yl ester;
Quinolin-3-yl-carbamic acid dydrogesterone-11β-yl ester, and
Quinolin-6-yl-carbamic acid dydrogesterone-11β-yl ester.

15. A pharmaceutical composition comprising a compound as claimed in claim 1 and at least one pharmaceutically acceptable carrier or pharmaceutical auxiliary substance.

16. A pharmaceutical composition as claimed in claim 15, further comprising at least one natural or synthetic estrogen or pro-drug thereof.

17. A pharmaceutical composition as claimed in claim 15, wherein said composition is in the form of an intrauterine device, a transdermal patch or a gel.

18. A method of modulating activity of a progesterone receptor in a subject, said method comprising administering to said subject an effective progesterone receptor modulating amount of a compound as claimed in claim 1.

19. A method of treating or inhibiting a condition selected from the group consisting of endometriosis, uterine fibroids, uterine leiomyoma, endometrial hyperplasia, dysmenorrhea, dysfunctional uterine bleeding, menorrhagia, metrorrhagia, hypermenorrhea, hot flushes, mood disorders, meningiomas, hormone-dependent cancer, female osteoporosis, Cushing's syndrome, major depression, neurodegenerative diseases, Alzheimer's disease, and demyelinating diseases, in a subject, said method comprising administering to said subject an effective progesterone receptor modulating amount of a compound as claimed in claim 1.

20. A method as claimed in claim 19, wherein said condition is a hormone-dependent cancer selected from the group consisting of female sex steroid dependent cancer, ovarian cancer, breast cancer, endometrial cancer, and prostate cancer.

21. A method of birth control, or modulating fertility, or effecting hormone replacement therapy in a female subject, said method comprising administering to said subject a pharmacologically effective amount of a compound as claimed in claim 1.

22. A method of contraception in an individual, said method comprising administering to said individual a pharmaceutically effective amount of a compound as claimed in claim 1.

23. A method of determining the presence of a progesterone receptor in a cell or cell extract, said method comprising:
(a) providing a compound as claimed in claim 1 labeled with a detectable label;
(b) contacting the cell or cell extract with the labeled compound;
(c) separating unbound labeled compound from the contacted cell or cell extract; and
(d) thereafter testing the contacted cell or cell extract to determine the presence labeled compound;
whereby the presence of labeled compound indicates the presence of a progesterone receptor.
